(19) United States
(12) Patent Application Publication
(10) Pub. No.: US 2025/0027048 A1
(43) Pub. Date: Jan. 23, 2025

(54) Genetically Modified Hepatocyte Populations

(71) Applicant: Cytotheryx, Inc., Rochester, MN (US)

(72) Inventors: Garrett Heffner, Rochester, MN (US); Raymond Hickey, Rochester, MN (US); Michael Holmes, Rochester, MN (US); Charity Juang, Rochester, MN (US); Whitney Krey, Rochester, MN (US); Glen Mikesell, Rochester, MN (US); Karen Vo, Rochester, MN (US); Fei Yi, Rochester, MN (US)

(21) Appl. No.: 18/274,152

(22) PCT Filed: Jan. 25, 2022
(86) PCT No.: PCT/US2022/013718

§ 371 Date: Jul. 25, 2023

Related U.S. Application Data
(60) Provisional application No. 63/141,769, filed on Jan. 26, 2021.

(51) Int. Cl. C12N 5/071; A01K 67/0271; A61K 35/407; C07K 14/705; C07K 14/74

(52) U.S. Cl. CPC C12N 5/067; A01K 67/0271; A61K 35/407; C07K 14/70503; C07K 14/70539; A01K 2207/12; A01K 2227/105; C12N 2510/00

(57) ABSTRACT

The present disclosure provides populations of genetically modified hepatocytes and/or hepatocyte progenitors and methods of producing the same. Methods of using said populations of genetically modified hepatocytes and/or progenitors, such as, but not limited to, treating a subject or a plurality of subjects for a condition or a plurality of conditions, are also provided. In some instances, genetically modified hepatocytes and/or hepatocyte progenitors of the population are hypoimmunogenic and the methods include methods of generating hypoimmunogenic hepatocytes and/or progenitors thereof. Non-human mammals containing engrafted populations of genetically modified hepatocytes and/or hepatocyte progenitors are also provided. Useful kits, systems, reagents, cells, and cell therapy doses are also provided.

### CROSS-REFERENCE TO RELATED APPLICATIONS

This application claims the benefit of U.S. Provisional Patent Application No. 63/141,769, filed Jan. 26, 2021, which application is incorporated herein by reference in its entirety.

### BACKGROUND

There are over 100,000 patients with acute liver disease and over half a million patients with decompensated liver cirrhosis in the United States alone. Liver disease accounts for 62,000 deaths annually in the US and approximately 2 million deaths worldwide, with 1.3 million due to cirrhosis specifically. As of 2019, cirrhosis was the 11th most common cause of death globally and the 12th leading cause in the US. Worldwide about 2 billion people consume alcohol, another approximately 2 billion adults are obese or overweight, and 400 million adults have diabetes. Alcohol consumption, liver lipid deposition, and insulin resistance are all considered to be major risk factors in the development of fibrosis and eventually cirrhosis. Moreover, while drug-induced liver injury continues to increase as a major cause of acute hepatitis, the global prevalence of viral hepatitis remains high.

In addition, in the developed world where communicable disease mortality is infrequent, genetic disorders, although individually rare, collectively represent a significant burden of childhood disease, disability, and mortality. Monogenic diseases are estimated to affect up to 6% of people at some point in their lives. Genetic disorders broadly include those attributable to a single genetic locus (i.e., “single gene disorders”, including monogenic diseases) as well as polygenic disorders attributable to a collection of multiple genetic risk factors and/or a combination with certain environmental factors. Quantifying the total burden of genetic diseases is difficult and, while many causative loci are known, genetic counseling has only had a minimal impact in reducing overall prevalence by perhaps 5% (despite being highly effective for certain conditions when screening applied diligently) (see e.g., Blencowe et al. J Community Genet. 2018). Genetic diseases include many rare liver diseases such as phenylketonuria, ornithine transcarbamylase deficiency, arginase-1 deficiency, α-1 antitrypsin deficiency, mucopolysaccharidosis, hemophilia A, hemophilia B, and the like. The large collective burden of genetic diseases, coupled with the low impact genetic counseling has had in reducing that burden, exemplifies the substantial ongoing impact these diseases have on global health.

Liver transplantation, when available and successful, is a life changing therapy that represents the second most common solid organ transplantation. Liver transplant is useful in both acquired and genetic liver disease. However, suitable livers are often not available in needed quantities or in time for subjects with rapidly declining conditions such as acute liver failure. In comparison to the expansive disease prevalences described above, less than 9,000 liver transplantations are performed in the US annually.

Despite recent advances in immunosuppressive agents and treatment protocols employing immunosuppressants, rejection remains a common complication of liver transplant. By some measures, incidence of acute allograft rejection ranges from 20% to 40% of liver transplants. Methods of predicting liver transplant rejection, as well as morbidity and mortality following transplant, are preliminary and the predictive power of such methods is controversial. The incidence of acute and chronic rejection has declined with improvement of immunosuppression regimens in liver transplant recipients. While acute rejection usually responds well to improved regimens, chronic rejection is a more difficult situation as a significant proportion of patients do not respond to increased immunosuppression, often leading to re-transplantation or death. Also, despite the advances due to improved immunosuppression regimens, many patients cannot tolerate immunosuppressants due to comorbidities or cannot be safely administered immunosuppressants due to existing contraindications.

### SUMMARY

The present disclosure provides populations of genetically modified hepatocytes and/or hepatocyte progenitors and methods of producing the same. Methods of using said populations of genetically modified hepatocytes and/or progenitors, such as, but not limited to, treating a subject or a plurality of subjects for a condition or a plurality of conditions, are also provided. In some instances, genetically modified hepatocytes and/or hepatocyte progenitors of the population are hypoimmunogenic and the methods include methods of generating hypoimmunogenic hepatocytes and/or progenitors thereof. Non-human mammals containing engrafted populations of genetically modified hepatocytes and/or hepatocyte progenitors are also provided. Useful kits, systems, reagents, cells, and cell therapy doses are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 is a graph showing target locus editing efficiencies in hepatocyte cell populations contacted with editing compositions targeting either beta-2-microglobulin (B2M) exon 1 or control AAVS1, with or without B2M-HLA-E or CD47 transgene delivery reagents, as measured by indels (left y-axis, speckled bars) and knock-out (KO) scores (left y-axis, hashed bars). Also provided is the percentage of cells having B2M KO (“% B2M− cells”) as measured by flow cytometry (right y-axis, black dots) in the corresponding hepatocyte cell populations following the described genetic modification.

FIG. 2 is a graph depicting the percentages of live cells having: only CD47 transgene genetic modification (% CD47”); both B2M KO and CD47 transgene genetic modifications (“% B2M−/CD47+”); only B2M-human leukocyte antigen E (HLA-E) fusion transgene genetic modification (“% HLA-E”); and both B2M KO and B2M-HLA-E fusion transgene genetic modification (“% B2M/HLA-E”), resulting from hepatocyte cell populations contacted with editing compositions targeting B2M exon 1 or control AAV1 with or without B2M-HLA-E or CD47 transgene delivery reagents as measured by flow cytometry. Also provided is the percentage of cells of each test group having B2M KO (“% B2M-”, dots).

FIG. 3A-3D is a series of grafts depicting the percentages of edited cells in input and output populations having B2M KO as measured by DNA analysis (FIG. 3A), B2M KO by flow cytometric analysis (FIG. 3B), HLA-E transgene expression by flow cytometric analysis (FIG. 3C), and double modification (i.e., both B2M KO and transgene expression) by flow cytometric analysis (FIG. 3D). Samples from, no-treatment-control (NTC) animals (i.e., animals transplanted with unmodified PHH) were also assessed in parallel.

FIG. 4 is a matrix of bioluminescent images collected at three times points (day 57 or 60, day 85, and day 97) after transplantation of Factor IX lentiviral vector transduced (LV-F9) or luciferase lentiviral vector transduced (LV-Luc) hepatocytes into recipient mice.

FIG. 5 represents quantification at all time points of the bioluminescent signal detected in LV-F9 and LV-Luc mice shown in FIG. 4.

FIG. 6 is a graph depicting the levels of human albumin, produced by transplanted engineered hepatocytes, measured in peripheral blood samples collected from LV-F9 and LV-Luc mice at 14, 28, 47, and 98 days following transplantation.

FIG. 7 is a graph depicting the levels of human Factor IX detected in peripheral blood samples collected from LV-F9 and LV-Luc mice at 14, 28, 47, and 98 days following transplantation. Reference levels indicating the limit of detection (LOD), the corresponding therapeutic level of Factor IX, and the corresponding normal physiological level of Factor IX are provided for comparison.

FIG. 8 is a plot of human Factor IX levels measured in each animal versus the corresponding human albumin level in each animal at day 47 after transplantation in LV-F9 and LV-Luc mice. Reference levels for 0.1%, 1%, and 5% engraftment as well as for 5% and 100% of normal physiological human Factor XI are shown as vertical and horizontal dotted lines, respectively.

FIG. 9 is a plot of human Factor IX levels measured in each animal versus the corresponding human albumin level in each animal at day 98 after transplantation in LV-F9 and LV-Luc mice. Reference levels for 0.1%, 1%, and 5% engraftment as well as for 5% and 100% of normal physiological human Factor XI are shown as vertical and horizontal dotted lines, respectively.

### DETAILED DESCRIPTION

The present disclosure provides populations of genetically modified hepatocytes and/or hepatocyte progenitors and methods of producing the same. Methods of using said populations of genetically modified hepatocytes and/or progenitors, such as, but not limited to, treating a subject or a plurality of subjects for a condition or a plurality of conditions, are also provided. In some instances, genetically modified hepatocytes and/or hepatocyte progenitors of the population are hypoimmunogenic and the methods include methods of generating hypoimmunogenic hepatocytes and/or progenitors thereof. Non-human mammals containing engrafted populations of genetically modified hepatocytes and/or hepatocyte progenitors are also provided. Useful kits, systems, reagents, cells, and cell therapy doses are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Definitions

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a,” “an,” or “the” include plural referents, unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes values from 9 to 11, including the values of 9, 10, and 11. The term “about” in relation to a reference numerical value can also include a range of values plus or minus 10%, 9%, 8%, 7%, 6%, 5%, 4%, 3%, 2%, or 1% from that value.

Before describing specific embodiments of the disclosure, it will be helpful to set forth definitions that are used in describing the present disclosure.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute.

The terms “control”, “control assay”, “control sample” and the like, refer to a sample, test, or other portion of an experimental or diagnostic procedure or experimental design for which an expected result is known with high certainty, e.g., in order to indicate whether the results obtained from associated experimental samples are reliable, indicate to what degree of confidence associated experimental results indicate a true result, and/or to allow for the calibration of experimental results. For example, in some instances, a control may be a “negative control” assay such that an essential component of the assay is excluded such that an experimenter may have high certainty that the negative control assay will not produce a positive result. In some instances, a control may be “positive control” such that all components of a particular assay are characterized and known, when combined, to produce a particular result in the assay being performed such that an experimenter may have high certainty that the positive control assay will not produce a positive result. Controls may also include “blank” samples, “standard” samples (e.g., “gold standard” samples), validated samples, etc.

The terms “recipient”, “individual”, “subject”, “host”, and “patient”, are used interchangeably herein and refer to any mammalian subject for whom diagnosis, treatment, or therapy is desired, indicated, or has been performed, such as human subjects. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human. In some cases, the methods of the disclosure find use in experimental animals, in veterinary application, and/or in the development of animal models, including, but not limited to, rodents including mice, rats, and hamsters; rabbits, dogs, cats, non-human primates, and other animals.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. For example, a preventative treatment, i.e. a prophylactic treatment, may include a treatment that effectively prevents a condition (e.g., a liver condition) or a treatment that effectively prevents or controls progression of a condition (e.g., a liver condition). In some instances, the treatment may result in a treatment response, such as a complete response or a partial response. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s).

Those in need of treatment can include those already afflicted (e.g., those with a condition, those with a liver condition (e.g., acute liver condition, chronic liver condition, etc.), those with cirrhosis, those with fibrosis, those with a disease, those with a monogenic disease, etc.) as well as those in which prevention is desired (e.g., those with increased susceptibility to a condition (e.g., a liver condition); those suspected of having a condition (e.g., a liver condition); those with an increased risk of developing a condition (e.g., a liver condition); those with increased environmental exposure to practices or agents causing a condition (e.g., a liver condition); those suspected of having a genetic or behavioral predisposition to a condition (e.g., a liver condition); those with a condition (e.g., a liver condition); those having results from screening indicating an increased risk of a condition (e.g., a liver condition); those having tested positive for a condition (e.g., a liver condition); those having tested positive for one or more biomarkers of a condition (e.g., a liver condition), etc.).

A therapeutic treatment is one in which the subject is afflicted prior to administration and a prophylactic treatment is one in which the subject is not afflicted prior to administration. In some embodiments, the subject has an increased likelihood of becoming afflicted or is suspected of having an increased likelihood of becoming afflicted (e.g., relative to a standard, e.g., relative to the average individual, e.g., a subject may have a genetic predisposition to a condition and/or a family history indicating increased risk), in which case the treatment can be a prophylactic treatment.

The term “recombinant”, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide, means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector). Recombinant nucleic acids, polynucleotides, cells, and the like may be referred to herein as engineered nucleic acids, engineered polynucleotides, engineered cells, and the like.

The terms “nucleic acid” and “polynucleotide” as used interchangeably herein refer to a polymeric form of nucleotides of any length, either ribonucleotides or deoxyribonucleotides, including analogs thereof. The terms refer only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural, or derivatized nucleotide bases, oligonucleotides containing modified or non-natural nucleotide bases (e.g., locked-nucleic acids (LNA) oligonucleotides), and interfering RNAs. In some instances, a polynucleotide may be a continuous open reading frame polynucleotide that excludes at least some non-coding sequence from a corresponding sequence present in the genome of an organism.

The term “polypeptide” is used interchangeably with the terms “polypeptides” and “protein(s),” and refers to a polymer of amino acid residues. Polypeptides include functional protein fragments of essentially any length as well as full length proteins. The term “peptide”, as used herein, will generally refer to a polypeptide chain of 40 or less amino acids. A “peptide therapeutic” is a peptide having an established therapeutic function. A “therapeutic polypeptide” is a polypeptide having an established therapeutic function. In some embodiments, polypeptides and peptides, including therapeutic polypeptides and peptides, may be expressed from a transgene.

The term “transduction”, as used herein, generally refers to the introduction of foreign nucleic acid into a cell using a viral vector and the term “transfection”, as used herein, generally refers to the process of introducing nucleic acid into cells by non-viral methods. However, in some instances throughout the disclosure, which will be readily apparent to the ordinarily skilled artisan, the terms “transduction” and “transfection” may be used interchangeably. In some instances, use of the term transduction may exclude non-viral delivery of nucleic acids. In some instances, use of the term transfection may exclude viral delivery of nucleic acids.

The terms “virus particles”, “virus”, and the like, refer to an infectious viral agent, including, e.g., baculovirus particles, lentivirus particles, adenovirus particles, and the like. Virus and virus particles may be naturally occurring, recombinant, engineered, or synthetic.

A “vector” is capable of transferring gene sequences to target cells. Typically, “vector construct”, “expression vector”, and “gene transfer vector” mean any nucleic acid construct capable of directing the expression of a gene of interest or other desired expression product and which can transfer nucleic acid sequences to target cells. Thus, the term includes cloning, and expression vehicles, as well as integrating vectors. A “vector” or “expression vector” may also refer to a replicon, such as plasmid, phage, virus, or cosmid, to which another nucleic acid segment, i.e. an “insert”, may be attached so as to bring about the expression and/or replication of the attached segment in a cell.

As used herein, the term “retrovirus” refers to an RNA virus that reverse transcribes its genomic RNA into a linear double-stranded DNA copy and subsequently covalently integrates its genomic DNA into a host genome. Retroviruses are a common tool for gene delivery. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immunodeficiency virus (BIV); and simian immunodeficiency virus (SIV). In some embodiments, HIV based vector backbones (i.e., HIV cis-acting sequence elements) may be employed.

Retroviral vectors, and more particularly, lentiviral vectors, may be used as described herein. The terms “retrovirus” or “retroviral vector,” as used herein are meant to include “lentivirus” and “lentiviral vectors” respectively. In addition, where reference is made to a specific type of retrovirus or vector, e.g., lentivirus or lentiviral vector, a skilled artisan will readily understand that, in some instances, other retroviruses and/or other retroviral vectors and/or retrovirus generally and/or retroviral vectors generally may be substituted for the specifically recited virus or vector.

The term “bioreactor”, as used herein, generally refers to an apparatus, machine, or system for the production under controlled conditions of living organisms or cells, or products synthesized and collected therefrom. Bioreactors may be manufactured, such as single-use or reusable vessels made of steel, glass, plastic, or other materials, and configured to maintain a controlled, and optionally homogeneous, environment appropriate for the desired biological activity. Manufactured bioreactors may include various control mechanisms, including but not limited to e.g., temperature controllers, pH controllers, gas controllers and exchangers (e.g., for controlling oxygen, carbon dioxide, and/or other gas levels), and the like, which may include combinations of sensors and actuators to read a particular signal and drive the signaled adjustment. Non-limiting examples of manufactured bioreactors include stirred-tank, rocker, air lift, fixed-bed, rotating wall, and perfusion bioreactors. Non-limiting examples of manufactured bioreactor components include agitators, impellers, spargers, probes, aseptic seals, baffles, feed lines, drain lines, air vents, heaters, coolers, and the like. Bioreactors may be employed to grow non-adherent as well as adherent cells. Bioreactors may range greatly in size, including but not limited to e.g., 15 mL volume or less to 2000 L volume or more, and may in some instances range from a liter or a few liters to 10, 20, 50, or 100 L or more. Further description of bioreactors, including examples of commercial suppliers, is provided by Stephenson et al. F1000Research (2018); the disclosure of which is incorporated herein by reference in its entirety. In addition to manufactured bioreactors, the term bioreactor also includes living animal or in vivo bioreactors.

The terms “living bioreactor”, “animal bioreactor”, and “in vivo bioreactor”, as used herein, generally refer to a living non-human animal, such as a non-human mammal, into which exogenous cells, such as hepatocyte-generating cells (i.e., cells that produce hepatocytes such as hepatocytes and/or hepatocyte progenitors), are introduced for engraftment and expansion. Animal bioreactors may be used to generate an expanded population of desired cells (which may include the introduced cells and/or their progeny), such as an expanded population of hepatocytes, generated from the introduced cells. Introduction of exogenous cells, such as hepatocyte-generating cells, into the bioreactor will generally involve xenotransplantation and, as such, the transplanted exogenous cells may, in some instances, be referred to as a xenograft, e.g., human-to-rodent xenograft, human-to-mouse xenograft, human-to-rat xenograft, human-to-porcine xenograft, mouse-to-rat xenograft, rat-to-mouse xenograft, rodent-to-porcine xenograft, etc. In some instances, allotransplantation into a bioreactor may be performed, e.g., rodent-to-rodent, porcine-to-porcine, etc., allotransplantations. A bioreactor may be configured, e.g., genetically and/or pharmacologically, to confer a selective advantage to introduced exogenous cells, such as introduced exogenous hepatocyte-generating cells, in order to promote engraftment and/or expansion thereof. Bioreactors may, in some instances, be configured to prevent rejection of introduced exogenous cells, including but not limited to e.g., through genetic and/or pharmacological immune suppression. As such, in vivo bioreactors may be subjected to external manipulation, e.g., through modulation of the animal's environment, diet, and/or the administration of one or more agents, e.g., to promote engraftment, to prevent rejection, to prevent infection, to maintain health, etc.

The term “ex vivo” is used to refer to handling, experimentation and/or measurements done in or on samples (e.g., tissue or cells, etc.) obtained from an organism, which handling, experimentation and/or measurements are done in an environment external to the organism. Thus, the term “ex vivo manipulation” as applied to cells refers to any handling of the cells (e.g., hepatocytes) outside of an organism, including but not limited to culturing the cells, making one or more genetic modifications to the cells and/or exposing the cells to one or more agents. Accordingly, ex vivo manipulation may be used herein to refer to treatment of cells that is performed outside of an animal, e.g., after such cells are obtained from an animal or organ (e.g., liver) thereof and before such cells are transplanted into an animal, such as an animal bioreactor or subject in need thereof. In contrast to “ex vivo”, the term “in vivo”, as used herein, may refer to cells that are within an animal, or an organ thereof, such as e.g., cells (e.g., hepatocytes and/or hepatocyte progenitors) that are within a subject, or the liver thereof, due to generation of the cells within the subject and/or transplantation of the cells into the subject.

As used herein, the term “collecting”, for example as it refers to expanded human hepatocytes, refers to the process of removing the expanded hepatocytes from an animal (e.g., non-human mammal, rodent, mouse, rat, or pig bioreactor) that has been injected or transplanted with isolated human hepatocytes, or other hepatocyte-generating cells, as described herein. In some instances, a non-human animal that receives a transplantation of cells, e.g., genetically modified cells, may also be referred to as a recipient animal. In some instances, a human subject that receives a transplantation, e.g., of expanded genetically modified hepatocytes, may be referred to as a treated subject, a recipient, or the like. Collecting optionally includes separating cells, e.g., hepatocytes, from other cell types, including but not limited to e.g., non-hepatic cells types (e.g., blood cells, extra-hepatic immune cells, vascular cells, etc.), non-hepatocyte hepatic cells (e.g., hepatic stellate cells, Kupffer cells, and liver sinusoidal endothelial cells).

As used herein, “cryopreserved” refers to a cell (such as a hepatocyte) or tissue that has been preserved or maintained by cooling to low sub-zero temperatures, such as 77 K or −196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10,370,638; 10,159,244; 9,078,430; 7,604,929; 6,136,525; and 5,795,711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

The term “survival” is used to refer to cells that continue to live, in vitro or in vivo, e.g., after some event, such as e.g., transplantation into an animal, co-culture with immune cells, contacting with a particular agent, etc. Cell survival may be assessed using a variety of methods, including direct assessments (such as e.g., qualitative or quantitative measurements of cell viability in a sample containing or expected to contain the cells of interest) and indirect assessments (such as e.g., qualitative or quantitative measurements of one or more functional consequences of the presence of the viable cells). Useful direct and indirect readouts of cell (e.g., hepatocyte) survival may include but are not limited to, cell counting (e.g., via hemocytometer, immunohistochemistry, flow cytometry, etc.), measuring a secreted factor or biomarker (e.g., via protein (e.g., albumin) ELISA, Western blot, etc.), assessing health of a recipient (for example by measuring vitals, function tests (e.g., liver function tests), etc.), and the like. The term “survival” is also used to refer to the length of time a subject, e.g., a subject with a liver disease or an animal model thereof, continues to live after some treatment, intervention, and/or challenge, such as e.g., administration or transplantation of cells (e.g., hepatocytes) to the subject, administration of a disease (e.g., liver disease) causing agent to the subject, withdrawal of an agent that inhibits, delays, avoids or prevents the development of disease (e.g., liver disease). Survival, as it refers to subject, may also be expressed in terms of the portion (e.g., percentage) of a population (e.g., a control or treatment group) that lives for a given period of time after some treatment, intervention, and/or challenge. One skilled in the biomedical arts will readily discern to what, e.g., cells or subjects, survival pertains as it is used herein.

The term “engraft” refers to the implantation of cells or tissues in an animal. As used herein, engraftment of human hepatocytes in a recipient animal refers to the process of human hepatocytes becoming implanted (e.g., in the liver) in the recipient animal following administration (e.g., injection). Under certain conditions engrafted human hepatocytes are capable of expansion in the recipient animal. As used herein, the term “expanding”, in relation to human hepatocytes, refers to the process of allowing cell division to occur such that the number of human hepatocytes increases. The term “in vivo expansion” refers to the process of allowing cell division of exogenous cells to occur within a living host (e.g., a non-human animal bioreactor, such as by way of example, a rodent (e.g., mouse or rat) bioreactor, a pig bioreactor, a rat bioreactor or the like, such that the number of exogenous cells increases within the living host. For example, human hepatocytes transplanted into a non-human animal bioreactor may undergo in vivo expansion within the bioreactor such that the number of human hepatocytes within the bioreactor increases.

The term “hepatocyte” refers to a type of cell that generally makes up 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

“Effective amount” or “amount effective to” refers to that amount of a compound and/or cells which, when administered (e.g., to a mammal, e.g., a human, or mammalian cells, e.g., human cells), is sufficient to effect the indicated outcome (e.g., engraftment, expansion, treatment, etc.). For example, an “effective amount”, such as a “therapeutically effective amount” refers to that amount of a compound and/or cells of the disclosure which, when administered to a mammal, e.g., a human, is sufficient to effect treatment in the mammal, e.g., human. The amount of a composition of the disclosure which constitutes a “therapeutically effective amount” will vary depending on the compound and/or cells, the condition and its severity, the manner of administration, and the age of the mammal to be treated, but can be determined routinely by one of ordinary skill in the art having regard to his or her own knowledge and to this disclosure.

### Methods, Compositions, Cell Populations, & Animals

The present disclosure includes methods, compositions, cell populations, and animals that include, generate, or are employed in making or using genetically engineered hepatocytes or progenitors thereof. Genetically modified hepatocytes of the present disclosure may include an integrated transgene that encodes for a gene product and/or an edited endogenous locus, including e.g., an ablation or “knock-out” of an endogenous locus or a gene, or portion of a gene (e.g., exon), therein. As described in more detail herein, essentially any gene product may be encoded by the transgene and/or essentially any locus may be targeted for an edit. Production of the genetically modified hepatocytes, and characteristics of the hepatocytes ultimately produced as well as cell populations that include the produced hepatocytes, will vary.

Until the studies described herein were performed, it was unknown whether genetically modified hepatocytes, such as those described herein, could be produced and expanded in the livers of a recipient in vivo bioreactors to generate therapeutic cell populations containing substantial numbers of hepatocytes with the desired genetic modification, as would be necessary for cell therapy. It remained unknown whether such cells, e.g., modified to encode a heterologous gene product and/or include the described genetic alterations, would efficiently engraft and repopulate production bioreactors, such as e.g., rat and pig bioreactors, to facilitate the generation of useful expanded populations that include substantial numbers of genetically modified hepatocytes.

In the xenotransplantation context, heterologous hepatocytes are generally at a survival disadvantage as compared to endogenous hepatocytes. In addition, genetic modification with gene editing reagents can negatively impact the cells of the population that are in fact edited, e.g., at one or more otherwise normal endogenous loci and/or to include an integrated transgene, leading to decreased proliferation, loss of cellular phenotype, increased cell fragility, and the like. These impacts can reduce the representation of the desired genetically modified cells within a cell population, including e.g., cell populations made or used for cell therapy or cell therapy production purposes. These negative impacts, alone or in combination with other processes such as host immune responses, can result in insufficient engraftment, expansion, recovery, and/or loss of transplanted edited cells when conventional techniques are employed due to, without being bound by theory, endogenous cells out-competing the introduced genetically modified cells even when host pre-conditioning is used to promote transplant engraftment. Accordingly, as shown herein, it was unexpectedly found that, through use of the herein described methods, cell populations containing substantial numbers of expanded hepatocytes carrying desired genetic modifications can be produced. Moreover, the percentage of hepatocytes having a desired genetic modification within engineered cell populations was surprisingly found to remain substantially constant before and after xenotransplantation and in vivo bioreactor expansion, indicating comparable fitness within a host of the unmodified and modified cells.

In some embodiments, genetically modified hepatocytes may be produced by contacting a cell population that contains hepatocytes, and/or hepatocyte progenitors, with an integrating vector that includes the transgene. The integrating vector, and the conditions under which the cells are contacted with the integrating vector, will generally be configured such that the transgene is functionally integrated into hepatocytes, or hepatocyte progenitors, of the cell population. In some embodiments, a transgene may be integrated by homology directed repair (HDR) or other DNA repair process, including e.g., where HDR or other repair process is facilitated through the use of a nuclease, such as but not limited to e.g., zinc-finger nucleases (ZFNs), TAL effector nucleases (TALENs), CRISPR associated (Cas) proteins, or the like.

To produce hypoimmunogenic hepatocytes, hepatocytes and/or progenitors thereof are genetically modified to include a transgene encoding a natural killer (NK) cell decoy receptor. Thus, specific examples are provided herein describing the functional integration of a transgene encoding a NK cell decoy receptor. However, this disclosure is not so limited and a skilled artisan will readily understand that any other sequence of interest may be used, e.g., to replace, modify, or add to, the described transgene to provide for functional integration of essentially any suitable and appropriate encoded gene product. As such, descriptions herein of specific transgenes encoding specific gene products, such as e.g., an NK decoy receptor, will be readily understood to also provide descriptions of the use of a transgene generically, encoding essentially any gene product.

For example, to produce engineered hepatocytes useful in treating a monogenic disease, hepatocytes and/or progenitors thereof are genetically modified to include a transgene encoding a functional version of the gene product disrupted in the monogenic disease. Nonlimiting examples of transgenes useful for functionally integrating into genetically modified hepatocytes, and/or hepatocyte progenitors, for treating monogenic diseases may include those transgenes encoding the full-length and/or modified and/or variant forms of: Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase-1, alpha-1 antitrypsin (AAT), fumarylacetoacetate hydrolase (FAH), the like, and combinations (including e.g., fusions and/or multi- or bicistronic versions) thereof.

By “functionally integrated”, as used herein, is generally meant that the transgene is integrated into the genome of the cell in such a way that the encoded gene product is expressed. Expression of the encoded gene product may be controlled, in whole or in part, by endogenous components of the cell or exogenous (including heterologous) components included in the transgene. For example, in some instances, expression of the encoded gene product may be controlled by one or more endogenous regulatory elements, e.g., promoter, enhancer, etc., at or near the genomic locus into which the transgene is inserted. In some instances, expression of the encoded gene product may be controlled by one or more exogenous (including heterologous) regulatory elements, e.g., promoter, enhancer, etc., present in the transgene, and operably linked to the encoded gene product, prior to insertion. Integration of a transgene renders a cell, such as a hepatocyte or a hepatocyte progenitor, genetically modified, e.g., producing genetically modified hepatocytes or genetically modified hepatocyte progenitors.

Functional integration of a transgene may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the targeted cells in a suitable transduction medium, at a suitable concentration (or multiplicity of infection), and for a suitable time for the vector to infect the target cells, facilitating functional integration of the transgene.

Suitable incubation and/or transduction (and/or transfection where applicable) times, e.g., in suitable medium, will vary. In some instances, a suitable incubation (or transduction and/or transfection) time may be 8 hours or less, less than 8 hours, 6 hours or less, less than 6 hours, 5 hour or less, less than 5 hours, 4 hours or less, less than 4 hours, 3 hours or less, less than 3 hours, 2 hours or less. In some instances, incubation (or transduction and/or transfection) may be performed with agitation. Various methods of agitation may be employed including, but not limited to e.g., rocking, such as e.g., horizontal rocking/shaking, nutation, and similar motions performed at suitable speed and transduction temperature, such as e.g., at or about 37 deg. C. In some instances, an incubation (or transduction and/or transfection) time of 8 hours or less, less than 8 hours, 6 hours or less, less than 6 hours, 5 hour or less, less than 5 hours, 4 hours or less, less than 4 hours, 3 hours or less, less than 3 hours, 2 hours or less may prevent, limit, or otherwise mitigate detrimental effects to the treated cells, e.g., resulting in increased numbers of desired genetically modified cells through enhanced transduction and/or transfection efficiency and/or improved viability (e.g., as compared to longer times).

In some embodiments, useful methods for functional integration of a transgene, and/or delivery of components of an editing composition as described herein, may include viral vectors. Viral vectors may be integrating or non-integrating. Non-limiting examples of useful viral vectors include retroviral vectors, lentiviral vectors, adenoviral (Ad) vectors, adeno-associated virus (AAV) vectors, hybrid Ad-AAV vector systems, and the like. Viral vectors may, in some instances, find use in other aspects of the herein described methods, such as e.g., delivery of gene editing components, such as e.g., nuclease (e.g., ZFN, TALEN, Cas protein, etc.) encoding nucleic acids, nuclease (e.g., ZFN, TALEN, Cas, etc.) proteins, Cas9 encoding nucleic acids, Cas9 proteins, guide RNAs (gRNAs), ribonucleoproteins (RNPs), and the like.

In some embodiments, useful methods for functional integration of a transgene, and/or delivery of components of an editing composition as described herein, may include non-viral vectors. Useful nonviral vectors will vary and generally refer to delivery means that do not employ viral particles and may generally be considered to fall into three categories: naked nucleic acid, particle based (e.g., nanoparticles), or chemical based. Non-limiting examples of nonviral vectors include lipoplexes (e.g., cationic lipid-based lipoplexes), emulsions (such as e.g., lipid nano emulsions), lipid nanoparticles (LNPs), solid lipid nanoparticles, peptide based vectors, polymer based vectors (e.g., polymersomes, polyplexes, polyethylenimine (PEI)-based vectors, chitosan-based vectors, poly (DL-Lactide) (PLA) and poly (DL-Lactide-co-glycoside) (PLGA)-based vectors, dendrimers, vinyl based polymers (e.g., polymethacrylate-based vectors), and the like), inorganic nanoparticles, and the like. Non-viral vectors may, in some instances, find use in other aspects of the herein described methods, such as e.g., delivery of gene editing components, such as e.g., nuclease (e.g., ZFN, TALEN, Cas protein, etc.) encoding nucleic acids, nuclease (e.g., ZFN, TALEN, Cas, etc.) proteins, Cas9 encoding nucleic acids, Cas9 proteins, gRNAs, RNPs, and the like.

Cell populations of the present disclosure will generally include hepatocytes and/or hepatocyte progenitors. In some instances, cell populations may be highly enriched for hepatocytes and/or hepatocyte progenitors. By “highly enriched”, it is meant that the cell type(s) of interest will be 70% or more, 75% or more, 80% or more, 85% or more, 90% or more of the cell composition, for example, about 95% or more, or 98% or more of the cell composition. In other words, the population may be a substantially pure composition of the cell type(s) of interest. In some instances, cell populations of interest may include crude preparations. In some instances, cell populations may be prepared from dissociated tissue, filtered or unfiltered. Cell populations containing hepatocytes and/or hepatocyte progenitors may, e.g., depending on the method of isolation and/or preparation, include or exclude various non-hepatocyte cell types including but not limited to e.g., hepatic non-parenchymal cells (NPCs), non-hepatocyte liver associated cells (e.g., stellate cells, Kupffer cells, endothelial cells, biliary cells, etc.), immune cells (e.g., WBCs), RBCs, etc. In some instances, cell populations may be pure or essentially pure preparations of hepatocytes and/or hepatocyte progenitors.

In some instances, cell populations may be prepared from one or more mammalian livers, such as e.g., human liver, non-human mammalian liver, rodent liver, rat liver, mouse liver, porcine liver, non-human primate (NHP) liver, or the like. In some instances, a cell population or multiple cell populations, or the engineered cells, including all the engineered cells of a population of multiple cell populations, may all be derived or prepared from a single human liver, such as a single cadaveric donor liver. The cells of a cell population may be all of one species (e.g., human, mouse, rat, pig, NHP, etc.) or may be a mixture of two or more species (i.e., a xenogeneic mixture). Xenogeneic cellular mixtures may include but are not limited to human cells mixed with non-human cells (such as e.g., human-rat mixtures, human-mouse mixtures, human-pig mixtures, human-NHP mixtures, rat-mouse mixtures, rat-pig mixtures, etc.). Sources of liver will vary and may include but are not limited to e.g., resected liver tissue, cadaveric human liver, chimeric (e.g., humanized) liver, bioreactor liver, and the like. Cell populations may be prepared from liver, including whole livers and liver portions, according to and/or including any convenient method, such as but not limited to e.g., dissociation, perfusion, filtration, sorting, and the like.

In some instances, all, or essentially all, of the cells of a cell population, including all or essentially all of the hepatocytes or human hepatocytes of a cell population, may be derived from a single donor liver or a portion of a single donor liver. In some instances, the cells of a cell population, including all or essentially all of the hepatocytes or human hepatocytes of a cell population, may be derived from a multiple different donor livers or portion of multiple different donor livers. In some instances, multiple cell populations may be derived from a single donor liver, including e.g., where the primary human hepatocytes collected from a single human donor liver are expanded many fold, including 2× or more, 5× or more, 10× or more, 20× or more, 50× or more, 100× or more, etc. to generate a plurality of cell populations, e.g., useful in treating a plurality of subjects.

In some instances, cell populations may be prepared from cultured hepatocytes and/or cultured hepatocyte progenitors. In some instances, cell populations may be prepared from primary hepatic cell preparations, including e.g., cell populations prepared from human liver that include primary human hepatocytes (PHH). In certain embodiments, the cell population may include hepatocytes isolated using standard techniques for any source, e.g., from human donors. In certain embodiments, the hepatocytes are PHH isolated from screened cadaveric donors, including fresh PHH or cryopreserved PHH. In some instances, PHH of a cell population have undergone no or a minimal number of cell cycles/divisions since isolation from a liver, including but not limited to e.g., 1 or less, 2 or less, 3 or less, 4 or less, 5 or less, 6 or less, 7 or less, 8 or less, 9 or less, 10 cycles/divisions or less.

In some instances, cell populations containing hepatocytes and/or hepatocyte progenitors may be prepared from cells that are not immortalized cell lines or not cells lines that are otherwise essentially perpetually propagated. For example, hepatocytes and/or hepatocyte progenitors of a cell population may be derived from primary liver cells and the progeny of primary liver cells, including e.g., the non-immortalized progeny of primary liver cells.

In some instances, cell populations may include, or may specifically exclude, hepatocyte progenitors. As used herein, the terms “hepatocyte progenitors” and “progenitors of hepatocytes” or the like, generally refer to cells from which hepatocytes are derived and/or cells that are differentiated into hepatocytes. In some instances, hepatocyte progenitors may be committed progenitors, meaning the progenitors will essentially only differentiate into hepatocytes. In some instances, hepatocyte progenitors may have varied potency and may be e.g., pluri-, multi-, or totipotent progenitors. Hepatocyte progenitors may include or be derived from stem cells, induced pluripotent stem cells (iPSCs), embryonic stem (ES) cells, hepatocyte-like cells (HLCs), and the like. In some instances, hepatocyte progenitors may be derived from mature hepatocytes and/or other non-hepatocyte cells, e.g., through dedifferentiation of hepatocytes and/or transdifferentiation of other hepatic or non-hepatic cell types.

The cells of a cell population, or subpopulation, of the present disclosure, including expanded cell populations of hepatocytes, may be derived or descended from multiple individual cells, including e.g., multiple individual hepatocytes obtained from a single donor or multiple individual hepatocytes obtained from multiple donors. Where a population of primary cell is derived from a single donor, such multiple individual cells share essentially the same donor genome but are, however, not clonally derived, not monoclonal, and may, in some instances, contain certain differences from one another, including e.g., different genetic variations, different epigenetic variations, different zonation in the donor liver, differences in gene expression, etc. Accordingly, in contrast to clonally-derived cell populations, cell populations expanded from a plurality of individual primary hepatocytes, including primary hepatocytes from a single donor or multiple donors, may be referred to as non-monoclonal or, in some instances, such expanded cells may be referred to as polyclonal or non-clonally expanded. In some instances, genetic modification of the present disclosure may be performed on a population individual primary hepatocytes (or the progeny thereof) to generate a non-monoclonal population of engineered hepatocytes and such cells may be expanded to generate an expanded population of non-monoclonal engineered hepatocytes. In some instances, a population of hepatocytes may be expanded to generate an essentially polyclonal population which is subsequently genetically modified to generate an expanded population of non-monoclonal engineered hepatocytes.

In some instances, the hepatocytes and/or hepatocyte progenitors, and/or the livers, subjects, and/or cell cultures from which such hepatocytes and/or hepatocyte progenitors are derived, may be healthy hepatocytes and/or hepatocyte progenitors. By “healthy hepatocytes and/or hepatocyte progenitors”, as used herein, is meant that the cells display a normal hepatocyte phenotype and/or genotype essentially free of functional and/or genetic deficiencies or defects in, or that would affect, normal liver and/or hepatocyte associated functions. Hepatocyte-associated functions include those functions primarily or exclusively carried out by hepatocytes in the liver, such as e.g., liver metabolism (e.g., hepatocyte metabolism), ammonia metabolism, amino acid metabolism (inc., bio-synthesis and/or catabolism), detoxification, liver protein (e.g., albumin, fibrinogen, prothrombin, clotting factor (e.g., factor V, VII, IX, X, XI, and XII), protein C, protein S, antithrombin, lipoprotein, ceruloplasmin, transferrin, complement protein) synthesis. Hepatocytes and/or hepatocyte progenitors may be healthy before, during, and/or after genetic modification(s) as described herein. For example, in some instances, a hepatocyte and/or hepatocyte progenitor may be a healthy cell prior to and after genetic modification, e.g., to functionally integrate a heterologous transgene and/or modify one or more endogenous loci, of the cell. In some instances, hepatocytes and/or hepatocyte progenitors are healthy following correction of a defective disease-associated allele or locus.

Healthy hepatocytes and/or hepatocyte progenitors will generally exclude those cells harboring a genetic aberration associated with a liver-associated monogenic disease, including but not limited to e.g., genetic cholestatic disorders, Wilson's disease, hereditary hemochromatosis, tyrosinemia, al antitrypsin deficiency, urea cycle disorders, Crigler-Najjar syndrome, familial amyloid polyneuropathy, primary hyperoxaluria type 1, atypical haemolytic uremic syndrome-1, and the like. Accordingly, healthy hepatocytes and/or hepatocyte progenitors may contain normal genes/alleles (i.e., non-disease associated genes/alleles, i.e., not contain disease-associated genes/alleles), at loci and/or genes corresponding with liver-associated monogenic diseases, such as but not limited to e.g., ABCB11 (BSEP), AGXT, ARG, ASL, ASS, ATP7B, ATP8B1 (aka FIC1), CFH, CPS, FAH, HAMP, HFE, JAG1, JH, MDR3 (ABCB4), NAGS, OTC, PI, SLC40A1, TFR2, TTR, UGT1A1, and the like. Further examples and description of genes corresponding with liver-associated monogenic diseases may be found in Fagiuoli et al. J Hepatol (2013) 59 (3): 595-612; the disclosure of which is incorporated herein by reference in its entirety. Cells harboring one or more genetic aberrations associated with a liver-associated monogenic disease may be referred to herein as “disease”, “diseased”, “disease-associated”, “dysfunctional”, or “defective” cells, or the like.

Cell populations, including hepatocytes and/or hepatocyte progenitors, may be manipulated in various ways outside of a living organism, i.e., ex vivo. Such manipulation may include, or specifically exclude in some cases, freezing, thawing, culturing, filtering, enriching, purifying, isolating, transfecting, transducing, and the like. In some instances, cells are thawed, if frozen, and placed in any suitable vessel or culture container. In some instances, cells are cultured in a suitable culture medium, with or without additional components.

Various suitable culture media can be used. In certain embodiments, the culture medium comprises a Hepatocyte Basal Media, FBS and/or a ROCK inhibitor, for example a 1:1 mix of Hepatocyte Basal Media and Lonza HCM™ Single Quots™, 5% FBS and 10 μM Rho kinase (ROCK) inhibitor. Various hepatocyte-compatible culture media are available, including but not limited to e.g., Liebovitz L-15, minimum essential medium (MEM), DMEM/F-12, RPMI 1640, Waymouth's MB 752/1 Williams Medium E, H 1777, Hepatocyte Thaw Medium (HTM), Cryopreserved Hepatocyte Recovery Medium (CHRM®), Human Hepatocyte Culture Medium (Millipore Sigma), Human Hepatocyte Plating Medium (Millipore Sigma), Human Hepatocyte Thawing Medium (Millipore Sigma), Lonza HCM™, Lonza HBM™, HepatoZYME-SFM (Thermo Fisher Scientific), Cellartis Power Primary HEP Medium (Cellartis), and the like. Various culture supplements and/or substrates may be included or excluded from a desired media, including but not limited to e.g., Lonza Single Quots™ supplements, HepExtend™ Supplement, fetal bovine serum, ROCK inhibitor, dexamethasone, insulin, HEGF, Hydrocortisone, L-glutamine, GlutaMAX™, buffer (e.g., HEPES, sodium bicarbonate buffers, etc.), transferrin, selenium complex, BSA, linoleic acid, collagen, collagenase, Geltrex™, methylcellulose, dimethyl sulfoxide, hyaluronidase, ascorbic acid, antibiotic, and the like. Hepatocyte-compatible media may be general use or specially formulated for primary, secondary, or immortalized hepatocytes and such media may contain serum or growth factors or configured to be serum-free, growth-factor-free, or with minimal/reduced growth factors.

In some instances, cell populations including hepatocytes and/or hepatocyte progenitors may be subjected to ex vivo manipulation, including but not limited to e.g., ex vivo manipulation as described in U.S. patent application Ser. No. 16/938,059 (US Pat. Pub. 20210024885) and PCT Patent Application No. PCT/US2020/043439 (WO2021/021612A1); the disclosures of which are incorporated herein by reference in their entirety. Such ex vivo manipulation may be, where performed, employed at various points in the herein described methods, such as but not limited to e.g., after isolation, before transplantation into a bioreactor, before administration to a subject (e.g., to treat the subject for a condition), and the like.

In some instances, freshly prepared hepatocytes and/or hepatocyte progenitors, or a cell population containing hepatocytes and/or hepatocyte progenitors, may be contacted with various reagents, compositions, and/or vectors, including e.g., a transgene encoding a gene product, editing compositions, and the like. Such freshly prepared cells may include freshly thawed cells (e.g., if previously cryopreserved), cells freshly isolated from a living subject (e.g., human, rodent, pig, etc.), cells freshly isolated from a liver or portion thereof (e.g., a cadaveric liver or portion thereof, a liver (or portion thereof) obtained from an in vivo bioreactor, etc.), or the like.

Cell populations may be generated that contain a plurality of genetically modified cells, including where such cells include a single genetic modification or multiple modifications. For example, in some instances, a cell population may be generated that includes a plurality of hepatocytes and/or hepatocyte progenitors that have been genetically modified to be hypoimmunogenic and thus the population may include a plurality of hypoimmunogenic hepatocytes and/or hepatocyte progenitors. The size of the plurality of cells with respect to the total cell population may vary. For example, in some instances, the plurality may comprise less than all of the cells of the population, including but not limited to e.g., where the plurality makes up at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the cell population. In some instances, a plurality of cells may make up all, or 100%, of a particular cell population.

In some instances, the cell population may include a plurality of cells modified to be hypoimmune where e.g., with respect to the total cell population the plurality makes up at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the cell population.

In some instances, the cell population may include a plurality of cells modified to include a particular transgene where e.g., with respect to the total cell population the plurality makes up at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the cell population.

In some instances, a cell population prior to and/or following expansion in a bioreactor may include at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of desired genetically modified hepatocytes. In some instances, before and after expansion in a bioreactor the input and output cell populations may each include a plurality of cells having a desired genetic modification, where the pluralities in the input and output populations may comprise percentages of the overall input and output populations that are within 30% or less, 25% or less, 20% or less, 15% or less, 10% or less, or 5% of less of one another.

Cell populations containing hepatocytes and/or hepatocyte progenitors, including genetically modified hepatocytes and/or hepatocyte progenitors, may be introduced, or transplanted, into subjects, including e.g., into human or non-human subjects for therapeutic purposes, non-human subjects for expansion and/or research purposes, and the like. When performed under sufficient conditions, hepatocytes and/or hepatocyte progenitors introduced into subjects may engraft, including engraft into the liver of the subject. In some instances, engraftment may be prevented, e.g., through the use of encapsulation techniques. Non-engrafting therapeutic cells may be delivered via various methods, including but not limited to e.g., application of encapsulated hepatocytes to the intraperitoneal space, the omental bursa, and/or other suitable location.

Any suitable approach for introducing the hepatocytes and/or hepatocyte progenitors into the liver may be employed. In some embodiments, introducing the hepatocytes and/or hepatocyte progenitors into the liver comprises delivering the hepatocytes to the spleen of the recipient. In one non-limiting example, the hepatocytes and/or hepatocyte progenitors may be introduced into the liver via splenic injection (e.g., laparotomy splenic injection or percutaneous splenic injection).

The present disclosure also includes non-human animals that include engrafted populations of hepatocyte and/or hepatocyte progenitor cells described herein, including where such engrafted cells are present in the liver of the non-human animal. For example, in some instances, a non-human animal may include an engrafted population of genetically modified hepatocytes and/or hepatocyte progenitors, including e.g., where the engrafted cells may be genetically modified to be hypoimmunogenic, include a therapeutic transgene, or both. Useful non-human animals include non-human mammals such as but not limited to e.g., rodents, murines (e.g., rats, mice), lagomorphs (e.g., rabbits), non-human primates, canines, felines, ungulates (e.g., equines, bovines, ovines, porcines, caprines), etc.

In some instances, a non-human animal may serve as an in vivo bioreactor. Cell populations that include hepatocytes and/or hepatocyte progenitors may be expanded by transplantation into an in vivo bioreactor and maintenance of the bioreactor under conditions suitable for expansion of the transplanted cells. Suitable in vivo bioreactors include but are not limited to e.g., rodent bioreactors, such as e.g., mouse bioreactors and rat bioreactors, pig bioreactors, and the like.

Animal bioreactors suitable for expansion of hepatocytes will vary. In certain embodiments, the animal is genetically modified at one or more loci. Genetic modifications may include knock-out or knock-down to generate an animal that is deficient at one or more loci or activation of one or more target genes. Genetic modifications may be made at multiple loci in any combination (one or more repressive modifications and/or one or more activating modifications). Useful genetic modifications in an in vivo bioreactor may include modifications in various genes including immune genes (e.g., resulting in immunodeficiency), liver function genes (e.g., resulting in liver function deficiency), metabolic genes (e.g., resulting in metabolic deficiency), amino acid catabolism genes (e.g., resulting in deficient amino acid catabolism and the like.

In certain embodiments, a useful genetically modified animal is a fumarylacetoacetate hydrolase (fah)-deficient animal, for example as described in U.S. Pat. Nos. 8,569,573; 9,000,257 and U.S. Patent Publication No. 20160249591, the disclosures of which are incorporated herein by reference in their entirety. FAH is a metabolic enzyme that catalyzes the last step of tyrosine catabolism. Animals having a homozygous deletion of the Fah gene exhibit altered liver mRNA expression and severe liver dysfunction. Point mutations in the Fah gene have also been shown to cause hepatic failure and postnatal lethality. Humans deficient for Fah develop the liver disease hereditary tyrosinemia type 1 (HT1) and develop liver failure. Fah deficiency leads to accumulation of fumarylacetoacetate, a potent oxidizing agent and this ultimately leads to cell death of hepatocytes deficient for Fah. Thus, Fah-deficient animals can be repopulated with hepatocytes from other species, including humans, containing a functional fah gene. Fah genomic, mRNA and protein sequences for a number of different species are publicly available, such as in the GenBank database (see, for example, Gene ID 29383 (rat Fah); Gene ID 14085 (mouse Fah); Gene ID 610140 (dog FAH); Gene ID 415482 (chicken FAH); Gene ID 100049804 (horse FAH); Gene ID 712716 (rhesus macaque FAH); Gene ID 100408895 (marmoset FAH); Gene ID 100589446 (gibbon FAH); Gene ID 467738 (chimpanzee FAH); and Gene ID 508721 (cow FAH)) and fah genomic loci in other species are readily identifiable through bioinformatics. Fah-deficient animals may include a genetically modified fah locus and may or may not include further genetic modifications at other loci, including for example where such an animal (e.g., mouse, pig or rat) is deficient in FAH, RAG-1 and/or RAG-2, and IL-2Rγ (referred in some instances as an “FRG” animal, such as an FRG mouse, FRG pig, or FRG rat).

Useful genetic modifications also include those resulting in immunodeficiency, e.g., from a lack of a specific molecular or cellular component of the immune system, functionality of a specific molecular or cellular component of the immune system, or the like. In some instances, useful genetic alterations include a genetic alteration of the Recombination activating gene 1 (Rag1) gene. Rag1 is a gene involved in activation of immunoglobulin V (D) J recombination. The RAG1 protein is involved in recognition of the DNA substrate, but stable binding and cleavage activity also requires RAG2. Rag-1-deficient animals have been shown to have no mature B and T lymphocytes. In some instances, useful genetic alterations include a genetic alteration of the Recombination activating gene 2 (Rag2) gene. Rag2 is a gene involved in recombination of immunoglobulin and T cell receptor loci. Animals deficient in the Rag2 gene are unable to undergo V (D) J recombination, resulting in a complete loss of functional T cells and B cells (see e.g., Shinkai et al. Cell 68:855-867, 1992). In some instances, useful genetic alterations include a genetic alteration of the common-gamma chain of the interleukin receptor (Il2rg). Il2rg is a gene encoding the common gamma chain of interleukin receptors. Il2rg is a component of the receptors for a number of interleukins, including IL-2, IL-4, IL-7 and IL-15 (see e.g., Di Santo et al. Proc. Natl. Acad. Sci. U.S.A. 92:377-381, 1995). Animals deficient in I12rg exhibit a reduction in B cells and T cells and lack natural killer cells. Il2rg may also be referred to as interleukin-2 receptor gamma chain.

In some instances, animals may be immunosuppressed, including e.g., where immunosuppression is achieved through administration of one or more immunosuppressive agents. Any suitable immunosuppressive agent or agents effective for achieving immunosuppression in the animal can be used. Examples of immunosuppressive agents include, but are not limited to, FK506, cyclosporin A, fludarabine, mycophenolate, prednisone, rapamycin and azathioprine. Combinations of immunosuppressive agents can also be administered. In some instances, immunosuppressive agents are employed in place of genetic immunodeficiency. In some instances, immunosuppressive agents are employed in combination with genetic immunodeficiency.

As summarized herein, genetically modified animals may include one or more (i.e., a combination of) genetic modifications. For example, such an animal may include a rag1 genetic modification, a rag2 genetic modification, a IL2rg genetic modification, or such an animal may include a rag1 or rag2 genetic modification and a genetic alteration of the I12rg gene such that the genetic alteration correspondingly results in loss of expression of functional RAG1 protein, RAG2 protein, IL-2rg protein, or RAG-1/RAG-2 protein and IL-2rg protein. In one example, the one or more genetic alterations include a genetic alteration of the Rag2 gene and a genetic alteration of the I12rg gene. In one example, the one or more genetic alterations include a genetic alteration of the Rag1 gene and a genetic alteration of the Il2rg gene. In some instances, useful genetic alterations include e.g., SCID, NOD, SIRPα, perforin, or nude. Altered loci may be genetic nulls (i.e., knockouts) or other modifications resulting in deficiencies in the gene product at the corresponding loci. Specific cells of the immune system (such as macrophages or NK cells) can also be depleted. Any convenient method of depleting particular cell types may be employed.

It will be appreciated that various models of liver injury, creating a selective growth advantage for hepatocyte xenografts, may be used in an animal bioreactor (e.g., rat, mouse, rabbit, pig) to facilitate hepatocyte engraftment and expansion, including, without limitation, inducible injury, selective embolism, transient ischemia, retrorsine, monocrotoline, thioacetamide, irradiation with gamma rays, carbon tetrachloride, and/or genetic modifications (e.g., Fah disruption, uPA, TK-NOG (Washburn et al., Gastroenterology, 140 (4): 1334-44, 2011), albumin AFC8, albumin diphtheria toxin, Wilson's Disease, and the like). Combinations of liver injury techniques may also be used.

In some embodiments, the animal is administered a vector (e.g., an Ad vector) encoding a urokinase gene (e.g., urokinase plasminogen activator (uPA)) prior to injection of the heterologous hepatocytes. Expression of uPA in hepatocytes causes hepatic injury and thus permits the selective expansion of hepatocyte xenografts upon transplantation. In one embodiment, the urokinase gene is human urokinase and may be secreted or non-secreted. See, e.g., U.S. Pat. Nos. 8,569,573; 9,000,257 and U.S. Patent Publication No. 20160249591.

In some instances, a TK-NOG liver injury model (i.e., an albumin thymidine kinase transgenic-NOD-SCID-interleukin common gamma chain knockout) may be used as the animal bioreactor as described herein. TK-NOG animals include a herpes simplex virus thymidine kinase hepatotoxic transgene that can be conditionally activated by administration of ganciclovir. Hepatic injury resulting from activation of the transgene during administration of ganciclovir provides a selective advantage to hepatocyte xenografts, facilitating use of such animals as in vivo bioreactors for the expansion of transplanted hepatocytes as described herein.

In some instances, an AFC8 liver injury model (characterized as having a FKBP-Caspase 8 gene driven by the albumin promoter) may be used as the animal bioreactor as described herein. AFC8 animals include a FK508-caspase 8 fusion hepatotoxic transgene that can be conditionally activated by administration of AP20187. Hepatic injury resulting from activation of the transgene during administration of AP20187 provides a selective advantage to hepatocyte xenografts, facilitating use of such animals as in vivo bioreactors for the expansion of transplanted hepatocytes as described herein.

In some instances, an NSG-PiZ liver injury model (characterized as having an α-1 antitrypsin (AAT) deficiency combined with immunodeficiency (NGS)) may be used as the animal bioreactor as described herein. NSG-PiZ animals have impaired secretion of AAT leading to the accumulation of misfolded PiZ mutant AAT protein triggering hepatocyte injury. Such hepatic injury provides a selective advantage to hepatocyte xenografts, facilitating use of such animals as in vivo bioreactors for the expansion of transplanted hepatocytes as described herein. The immunodeficiency renders the animal capable of hosting a xenograft without significant rejection.

In some instances, an animal may be preconditioned to improve the recipient liver's ability to support the transplanted cells. Various preconditioning regimens may be employed, including but not limited to e.g., irradiation preconditioning (e.g., partial liver irradiation), embolization preconditioning, ischemic preconditioning, chemical/viral preconditioning (using e.g., uPA, cyclophosphamide, doxorubicin, nitric oxide, retrorsine, monocrotaline, toxic bile salts, carbon tetrachloride, thioacetamide, and the like), liver resection preconditioning, and the like. In some instances, hepatocyte-generating cells may be introduced in the absence of preconditioning and/or a procedure will specifically exclude one, all, or some combination of preconditioning regimens or specific reagents, including e.g., one or more of those described herein. In some instances, induction of liver injury through cessation of NTBC or administration of ganciclovir or AP20187 may be used for preconditioning. Where employed, preconditioning may be performed at some time, including hours, days, or weeks or more, prior to transplantation of hepatocyte-generating cells, including e.g., at least 6 hours, at least 12 hours, at least 24 hours, at least 36 hours, at least 48 hours, at least 60 hours, at least 72 hours, at least 4 days, at least 5 days, at least 6 days, at least a week, or at least two weeks at least prior to transplantation.

After optional pre-conditioning (e.g., with uPA) of the animal (e.g., 24 hours after pre-conditioning), heterologous hepatocytes and/or hepatocyte progenitors can be delivered to the animal via any suitable method. In certain embodiments, the hepatocytes and/or hepatocyte progenitors as described herein are administered directly to the liver (e.g., via portal vein injection) and/or via intra-splenic injection where the hepatocytes and/or progenitors will travel through the vasculature to reach the liver. In certain embodiments, anywhere between 1×10⁵ and 1×10⁹ (e.g., 5×10⁵/mouse, 5-10×10⁶/rat, etc.) hepatocytes and/or hepatocyte progenitors are introduced into an animal (e.g., an FRG animal), optionally preconditioned (e.g., 24 hours prior to administration), e.g., with adenoviral uPA (e.g., 1.25×10⁹ PFU/25 grams of mouse body weight). The number of hepatocytes and/or hepatocyte progenitors introduced into the bioreactor will vary and may range, e.g., depending on various factors including the species and size of the animal receiving the cells, from 1×10⁵ or less to 1×10⁹ or more, including but not limited to e.g., 1×10⁵ to 1×10⁹, 1×10⁶ to 1×10⁹, 1×10⁷ to 1×10⁹, 1×10⁸ to 1×10⁹, 1×10⁵ to 1×10⁶, 1×10⁵ to 1×10⁷, 1×10⁵ to 1×10⁸, 1×10⁶ to 1×10⁷, 1×10⁷ to 1×10⁸, 1×10⁶ to 1×10⁸, etc. In some instances, the number of cells administered may be 1×10⁹ or less, including e.g., 0.5×10⁹ or less, 1×10⁸ or less, 0.5×10⁸ or less, 1×10⁷ or less, 0.5×10⁷ or less, 1×10⁶ or less, 0.5×10⁶ or less, 1×10⁵ or less, etc. Hepatocytes and/or hepatocyte progenitors introduced into a bioreactor (or non-human animal generally) may vary and such cells may be allogenic or heterologous with respect to the bioreactor (or non-human animal generally).

In addition, immune suppression drugs can optionally be given to the animals before, during and/or after the transplant to eliminate the host versus graft response in the animal (e.g., the mouse, pig, or rat) from a xenografted heterologous hepatocytes. In some instances, by cycling the animals off immune suppression agents for defined periods of time, the liver cells become quiescent and the engrafted cells will have a proliferative advantage leading to replacement of endogenous hepatocytes (e.g., mouse, pig, or rat hepatocytes) with heterologous hepatocytes (e.g., human hepatocytes). In the case of human hepatocytes, this generates animals with high levels of humanization of the liver, i.e., humanized livers. Heterologous hepatocyte repopulation levels can be determined through various measures, including but not limited to e.g., quantitation of human serum albumin levels, optionally correlated with immunohistochemistry of liver sections from transplanted animals.

In some embodiments, an agent that inhibits, delays, avoids or prevents the development of liver disease is administered to the animal bioreactor during the period of expansion of the administered hepatocytes. Administration of such an agent avoids (or prevents) liver dysfunction and/or death of the animal bioreactor (e.g., mouse, rat, or pig bioreactor) prior to repopulation of the animal bioreactor (e.g., mouse, rat, or pig bioreactor) with healthy (e.g., FAH-expressing) heterologous hepatocytes. The agent can be any compound or composition that inhibits liver disease in the disease model relevant to the bioreactor. One such agent is 2-(2-nitro-4-trifluoro-methyl-benzoyl)-1,3 cyclohexanedione (NTBC), but other pharmacologic inhibitors of phenylpyruvate dioxygenase, such as methyl-NTBC can be used. NTBC is administered to regulate the development of liver disease in a Fah-deficient animal. The dose, dosing schedule and method of administration can be adjusted, and/or cycled, as needed to avoid catastrophic liver dysfunction, while promoting expansion of hepatocyte xenografts, in the Fah-deficient animal bioreactor. In some embodiments, the Fah-deficient animal is administered NTBC for at least two days, at least three days, at least four days, at least five days or at least six days following transplantation of hepatocytes as described herein. In some embodiments, the Fah-deficient animal is further administered NTBC for at least about one week, at least about two weeks, at least about three weeks, at least about four weeks, at least about one month, at least about two months, at least about three months, at least about four months, at least about five months, or at least about six months. In some embodiments, the NTBC (or another compound with a liver protective effect) is withdrawn at about two days, about three days, about four days, about five days, about six days or about seven days following hepatocyte transplantation.

The dose of NTBC administered to the Fah-deficient animal can vary. In some embodiments, the dose is about 0.5 mg/kg to about 30 mg/kg per day, e.g., from about 1 mg/kg to about 25 mg/kg, from about 10 mg/kg per day to about 20 mg/kg per day, or about 20 mg/kg per day. NTBC can be administered by any suitable means, such as, but limited to, in the drinking water, in the food or by injection. In one embodiment, the concentration of NTBC administered in the drinking water is about 1 to about 30 mg/L, e.g., from about 10 to about 25 mg/L, from about 15 to about 20 mg/L, or about 20 mg/L. In certain embodiments, NTBC administration is cyclical from before transplantation to 4 to 8 or more weeks post-transplantation.

Expanded hepatocytes derived from transplanted hepatocytes and/or hepatocyte progenitors can be collected from the animal bioreactor after any period of time, including but not limited to 7 to 180 days (or any day therebetween) or more after transplantation.

At the time of collection, the liver of the animal bioreactor may be repopulated with introduced hepatocytes, hepatocyte progenitors, and/or the progeny thereof (including e.g., genetically modified hepatocytes, hepatocyte progenitors, and/or the progeny thereof) to varying degrees. For example, in some instances, the liver of a repopulated animal may be at least 30% repopulated or more, including but not limited to e.g., at least 40%, at least 50%, at least 60%, at least 70%, at least 75%, at least 80%, at least 85%, at least 90%, or at least 95% repopulated. As such, the hepatocytes of a repopulated animal may, in some instances, include at least 30% or more genetically modified hepatocytes as described herein, including but not limited to e.g., at least 40%, at least 50%, at least 60%, at least 70%, at least 75%, at least 80%, at least 85%, at least 90%, or at least 95% genetically modified hepatocytes. Accordingly, collected cell populations may include similar percentages of genetically modified hepatocytes (including introduced cells (e.g., genetically modified hepatocytes and/or hepatocyte progenitors) and/or the progeny thereof), including e.g., 30% or more, 40% or more, 50% or more, 60% or more, 70% or more, 75% or more, 80% or more, 85% or more, 90% or more, or 95% or more genetically modified hepatocytes.

In certain embodiments, the expanded hepatocytes are collected 28 to 56 days (or any day therebetween) after transplantation. In some instances, hepatocytes are collected at 1 week, at 2 weeks or earlier, at 3 weeks or earlier, before 4 weeks, at 4 weeks or earlier, at 5 weeks or earlier, at 6 weeks or earlier, at 7 weeks or earlier, before 8 weeks, at 8 weeks or earlier, at 9 weeks or earlier, at 10 weeks or earlier, at 11 weeks or earlier, before 12 weeks, at 12 weeks or earlier, at 13 weeks or earlier, before 14 weeks, or at 14 weeks or earlier.

Furthermore, the expanded hepatocytes can be collected from the animal using any of a number of techniques. For example, the hepatocytes can be collected by enzymatic digestion of the animal's liver, followed by gentle mincing, filtration, and centrifugation. Furthermore, the hepatocytes can be separated from other cell types, tissue and/or debris using various methods, such as by using an antibody that specifically recognizes the cell type of the engrafted hepatocyte species. Such antibodies include, but are not limited to, an antibody that specifically binds to a class I major histocompatibility antigen, such as anti-human HLA-A, B, C (Markus et al. (1997) Cell Transplantation 6:455-462). Antibody bound hepatocytes can then be separated by panning (which utilizes a monoclonal antibody attached to a solid matrix), fluorescence activated cell sorting (FACS), magnetic bead separation, or the like. Alternative methods of collecting hepatocytes may also be employed.

In some instances, collected hepatocytes may be serially transplanted one or more times into additional animal bioreactors. Serial transplantations may be conducted two, three, four or more times in the same or different species of animal, for example using rats, pigs, mice or rabbits for all serial transplantations or alternatively, using any combination of suitable animal bioreactors for the serial transplantations (one or more in rats, one or more in pigs, etc.).

Furthermore, following collection of the hepatocytes from an animal bioreactor, the hepatocytes may be subjected to various genetic manipulations as described herein. For example, hepatocytes collected from a bioreactor may be genetically modified, e.g., by introduction of a transgene and/or editing of one or more genetic loci, prior to administration to a subject. Collected, and optionally isolated, expanded hepatocytes may be used fresh or may be cryopreserved before use.

In certain embodiments, hepatocytes and/or hepatocyte progenitors, including genetically modified hepatocytes and/or hepatocyte progenitors, may be encapsulated. Hepatocytes and/or progenitors thereof may be encapsulated using any method, typically prior to administration to a subject. See, e.g., Jitraruch et al. (2014) PLOS One 9:10; Dhawan et al. (2020) J Hepatol. 72 (5): 877-884; Bochenek et al. (2018) Nature Biomedical Engineering 2:810-821. Cell encapsulation within semi-permeable hydrogels represents a local immuno-isolation strategy for cell-based therapies without the need for systemic immunosuppression. The hydrogel sphere facilitates the diffusion of substrates, nutrients, and proteins necessary for cell function while excluding immune cells that would reject allogeneic cells. Alginate spheres are one of the most widely investigated cell encapsulation materials because this anionic polysaccharide forms a hydrogel in the presence of divalent cations under cell-friendly conditions. In some instances, e.g., due to genetic modification rendering the hepatocytes and/or hepatocyte progenitors hypoimmunogenic, the cells may be administered without encapsulation, as such the cells may be used unencapsulated or naked.

Also provided herein is a decellularized liver, or other acellularized scaffold (including natural and synthetic scaffolds), seeded and/or repopulated with a population of hepatocytes and/or hepatocyte progenitors produced by the methods as described herein. For example, a cell population that includes genetically modified hepatocytes and/or progenitors thereof as described herein may be introduced (with or without other supporting cell types) into a decellularized liver, or portion thereof or other acellularized scaffold, which is subsequently maintained under conditions sufficient for repopulation of the decellularized liver, or portion thereof by hepatocytes of and/or generated from cell population.

A liver, such as a human liver or non-human mammal such as a pig, or portion thereof may be obtained, and optionally surgically processed (e.g., to isolate one or more portions or lobe(s) of the liver). The liver, or portion thereof, is then decellularized by any convenient and appropriate means, including e.g., mechanical cell damage, freeze/thawing, cannulation and retrograde profusion of one or more decellularization reagents (e.g., one or more protease (e.g. trypsin), one or more nuclease (e.g., DNase), one or more surfactants (e.g., sodium dodecyl sulfate, Triton X-100, or the like), one or more hypotonic reagents, one or more hypertonic reagents, combinations thereof, or the like. The decellularized liver, or a portion thereof, may be stored and/or presoaked in a hepatocyte-compatible media. Cell suspension containing ex vivo manipulated hepatocyte-generating cells as described herein may then be applied to the decellularized liver, or portion thereof, by any convenient mechanism, such as e.g., injection, perfusion, topical application (e.g., drop-by-drop), or combination thereof. In some instances, the ex vivo manipulated hepatocyte-generating cells may be present in the cell suspension, for seeding into a prepared scaffold, at any convenient and appropriate concentration, including e.g., a concentration of 1×10⁵ or less to 1×10⁷ or more cells per 50 μL, including but not limited to e.g., 1-2×10⁶ cells per 50 μL. Seeded decellularized liver, portions thereof, and/or other acellularized scaffolds may be maintained under suitable conditions for engraftment/attachment and/or expansion of the introduced cells, where such conditions may include suitable humidity, temperature, gas exchange, nutrients, etc. In some instances, a seeded liver, portion thereof, and/or other acellularized scaffold may be maintained in a suitable culture medium a humid environment at or about 37° C. with 5% CO₂. Following attachment and/or expansion of seeded and/or generated hepatocytes to or within the decellularized liver, portion thereof, or other acellularized scaffold, the material may be employed for various uses, including e.g., transplantation into a subject in need thereof, such as a human subject with decreased liver function and/or a liver disease. Methods and reagents relating to decellularization of liver, including human livers, and the production of hepatocyte-receptive acellular scaffolds are described in e.g., Mazza et al. Sci Rep 5, 13079 (2015); Mango et al. Adv. Funct. Mater. 2000097 (2020); Shimoda et al. Sci Rep 9, 1543 (2019); Croce et al. Biomolecules. 2019, 9 (12): 813; as well as U.S. Pat. No. 10,688,221, the disclosures of which are incorporated herein by reference in their entirety.

Collected cell populations produced by the methods as described herein and therapeutic or pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of hepatocytes and pharmaceutical compositions may be prepared and/or used fresh or may be cryopreserved. In some instances, populations of hepatocytes and pharmaceutical compositions thereof may be prepared in a “ready-to-use” format, including e.g., where the cells are present in a suitable diluent and/or at a desired delivery concentration (e.g., in unit dosage form) or a concentration that can be readily diluted to a desired delivery concentration (e.g., with a suitable diluent or media). Populations of hepatocytes and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure includes a plurality of cell therapy doses, e.g., each contained in suitable container, including e.g., where the genetically modified hepatocytes of the plurality of doses are all derived, including expanded, from a hepatocyte population, e.g., a master cell bank, created from a single human donor liver. In some instances, the present disclosure includes a plurality of cell therapy doses, e.g., each contained in suitable container, including e.g., where the genetically modified hepatocytes of the plurality of doses are all derived, including expanded, from a single hepatocyte population, e.g., a master cell bank, created from a plurality (e.g., 2, 2 or more, 3 or less, 3, 3 or more, 4 or less, 4, 4 or more, 5 or less, 5, 5 or more, 6 or less, 6, 6 or more, 7 or less, 7, 7 or more, 8 or less, 8, 8 or more, 9 or less, 9, 9 or more, 10 or less, 10 or more, etc.) human donor livers.

Pluralities of cell therapy doses may be generated through a variety of methods. In some instances, human hepatocytes are genetically modified and the genetically modified hepatocytes are expanded in one or more in vivo bioreactors to generate an expanded population of genetically modified human hepatocytes used in formulating the plurality of doses. In some instances, expanded human hepatocytes obtained from one or more in vivo bioreactors are genetically modified to generate an expanded population of genetically modified human hepatocytes used in formulating the plurality of doses. Aliquoting expanded populations of genetically modified human hepatocytes into pluralities of hepatocyte cell therapy doses may be performed by a variety of means and may result in various different total amounts of unit doses containing a variety of different numbers of hepatocytes. For example, in some instances at least 2 doses, including e.g., at least 3, 4, 5, 6, 7, 8, 9, 10, 20, 30, 40, 50, 60, 70, 80, 90, 100, 150, 200, 250, 500, 750, or 1000 unit doses may be generated, including e.g., where such doses each include e.g., at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least 40 billion, at least 50 billion, at least 60 billion, at least 70 billion, at least 80 billion, at least 90 billion, or at least 100 billion hepatocytes.

Methods of the present disclosure may include treating a plurality of subject with the herein described cell therapy doses, including where the hepatocytes contained in such doses are, e.g., derived from a single human donor liver or multiple human donor livers. For example, in some instances, e.g., wherein a plurality of doses includes at least 10 doses of at least 1 billion (or at least 10 billion) hepatocytes each, such a method may include treating 2, 3, 4, 5, 6, 7, 8, 9, or 10 separate subjects with the at least 10 doses. In some instances, e.g., wherein a plurality of doses includes at least 100 doses of at least 1 billion (or at least 10 billion) hepatocytes each, such a method may include treating at least 10, 20, 30, 40, 50, 60, 70, 80, 90, or 100 separate subjects subjects with the at least 100 doses. Such doses may be administered to subjects in need thereof, including multiple subjects with the same condition as well as multiple subjects with different conditions, to treat the subjects' conditions. Accordingly, through employing the methods described herein multiple subjects, and in some cases many subjects, in need of therapy may be treated using genetically modified hepatocytes derived and expanded from a population of cells collected from a single human donor liver.

As summarized above, genetic modification of hepatocytes and/or hepatocyte progenitors may include functional integration of a transgene encoding a gene product. Essentially any encoded gene product may be employed. Encoded gene products may be recombinant versions of proteins, including e.g., prokaryotic or eukaryotic proteins, such as mammalian (e.g., human, non-human primate, pig, rat, mouse, etc.) or non-mammalian proteins, protein fragments, peptides, synthetic proteins, fusion proteins, etc., or non-coding nucleic acids, and the like.

In some instances, hepatocytes and/or hepatocyte progenitors of a cell population may be genetically modified to express one or more immune inhibitory proteins, including e.g., T cell inhibitory proteins, NK cell inhibitory proteins, or the like. For example, in some instances, hepatocytes and/or hepatocyte progenitors may be genetically modified to include a transgene encoding a gene product that is an NK cell decoy receptor. The term “NK cell decoy receptor”, as used herein, generally refers to a mammalian (e.g., human) protein receptor or portion thereof, recombinant or synthetic receptor or portion thereof, or the like that, when expressed on the surface of a cell, provides protection from killing by NK cells, e.g., by serving as a ligand for an NK inhibitory receptor (such as e.g., KIRs, HLA-cl I-specific receptors, NKG2 inhibitory receptors (e.g., CD94/NKG2A heterodimer, NKG2B receptors), LIR-1, checkpoint receptors, SIRPα, PD-1 (CD279), Siglec-7 (CD328), IRP60 (CD300a), Tactile (CD96), IL1R8, TIGIT, TIM-3, NKG2A/KLRD1 (CD159a/CD94), KIR2DL1 (CD158a), KIR2DL2/3 (CD158b), (CD158d)a, KIR2DL5 (CD158f), KIR3DL1 (CD158e1), KIR3DL2 (CD158k), ILT2/LIR-1 (CD85J), LAG-3 (CD223), and the like). NK cell inhibitory receptors may be HLA-specific or non-HLA-specific and, as such, NK cell decoy receptors include both HLA derived polypeptides and non-HLA derived polypeptides.

Non-limiting examples of useful NK cell decoy receptors include but are not limited to e.g., HLA class I proteins and fragments thereof (e.g., HLA class 1a proteins (e.g., HLA-A, -B, -C) and fragments thereof, HLA class 1b proteins (HLA-E, -F, -G, -H) and fragments thereof), synthetic HLA class I protein fusions (including e.g., HLA class 1a fusions, HLA class 1b fusions, HLA class 1a/1b fusions, and the like), CD47, PD-L1 (CD274), PD-L2 (CD273), PVR (CD155), IL-37, Gal-9, PtdSer, HMGB1, CEACAM1, HLA-E, HLA-G, HLA-C1, HLA-C2, HLA-A-Bw4, HLA-B-Bw4, HLA-A*03, HLA-A*11, MHC-I proteins, MHC-2 proteins, CD80 and CD86 (CTLA-4), LSECtin (LAG3), CD112 (TIGIT), CXADR (JAML/AMICA1), HVEM (BTLA), and the like. Useful HLA genes, alleles, and the proteins thereof include e.g., those described in Marsh et al. (2010) Tissue Antigens 75:291-455; the disclosure of which is incorporated herein by reference in its entirety. In some instances, useful NK cell decoy receptors may include only a portion or fragment, e.g., of an exemplary NK cell decoy receptor protein described herein, or may include a fusion of two or more proteins and/or protein fragments, e.g., a fusion of two or more exemplary NK cell decoy receptor proteins described herein and/or fragments thereof.

In some instances, a useful NK cell decoy receptor may include an HLA class 1b fusion protein that includes e.g., a beta-2-microglobulin (B2M) protein or portion thereof fused, directly or in directly, to one or more of HLA-E, HLA-F, HLA-G, or HLA-H or one or more portions thereof. Useful HLA class 1b fusion proteins may or may not include a peptide antigen, optionally a cleavable peptide antigen e.g., that upon cleavage can occupy a peptide binding cleft of the fusion protein. Useful portions of B2M and/or HLA class Ib proteins that may be included in an HLA class 1b fusion protein include but are not necessarily limited to e.g., extracellular domains, transmembrane domains, cytoplasmic domains, signal peptides, signal sequences, alpha1 domains, alpha2 domains, alpha3 domains, alpha chains, and the like. In some instances, HLA class 1b fusion proteins may include one or more non-HLA and/or non-B2M portions (i.e., portions not derived from an HLA protein and/or a B2M protein) such as e.g., one or more linker portions, such as e.g., a synthetic linker, such as e.g., a glycine linker, a glycine-serine linker, or the like.

The following are provided as non-limiting examples of proteins useful, alone or in combination, in whole or in part(s), in various NK cell decoy receptors. An exemplary human B2M sequence (UniProtKB ID: P61769; NCBI RefSeq: NP_004039.1) is SEQ ID NO:032.

An exemplary human HLA-E sequence (UniProtKB ID: P13747; NCBI RefSeq: NP_005507.3) is SEQ ID NO:033.

An exemplary human HLA-G sequence (UniProtKB ID: P17693; NCBI RefSeq: NP_002118.1) is SEQ ID NO:034.

In some instances, useful B2M-HLA-E fusions include e.g., a full- or partial-length B2M fused to an HLA-E fragment, e.g., through a GS-linker, optionally with an HLA-G signal sequence, such as but not limited to e.g., SEQ ID NO:035.

In some instances, useful B2M-HLA-E fusions may include a signal sequence (e.g., optionally a B2M signal sequence), optionally with a cleavable HLA-G peptide joined via a linker to a full- or partial-length B2M sequence joined via a linker to an HLA-E fragment, such as but not limited to SEQ ID NO:036 (a coding sequence of which is also referred to herein as “B2M-HLA-E fusion”).

As will be readily apparent, other arrangements of full- or partial-length of HLA class I protein sequences as well as full- or partial-length B2M sequences, with or without other components or substitute components, such as linkers, signal sequences, peptide antigen sequences, etc. may be made and employed in HLA class I, class 1b, HLA-E-B2M, and HLA-E/G-B2M fusions and the like. In some instances, useful proteins, fusions, sequences, portions thereof, and the like may include those described in U.S. Patent App. Pub. No. US20140134195A1; the disclosure of which is incorporated herein by reference in its entirety.

An exemplary human CD47 (hCD47) sequence is SEQ ID NO:037.

An exemplary truncated hCD47 sequence is SEQ ID NO:038.

In some instances, as will be readily understood, useful sequences, including amino acid and nucleic acid sequences such as but not limited to those such sequences described herein, may be employed as described or may vary and, e.g., may include one or more substitutions, deletions, insertions, and/or truncations, or other modifications. For example, an amino acid sequence, such as but not limited to an amino acid sequence described herein, may include at least 1, 1, at least 2, 2 or fewer, at least 3, 3 or fewer, at least 4, 4 or fewer, at least 5, 5 or fewer, at least 6, 6 or fewer, at least 7, 7 or fewer, at least 8, 8 or fewer, at least 9, 9 or fewer, at least 10, 10 or fewer, or greater than 10 amino acid substitutions. In some instances, a nucleic acid sequence may have an alternative sequence that encodes for the same amino acid sequence, such as e.g., a codon optimized sequence. In some instances, one or more bases or one or more codons of a nucleic acid sequence may be modified to introduce one or more substitutions, such as e.g., at least 1, 1, at least 2, 2 or fewer, at least 3, 3 or fewer, at least 4, 4 or fewer, at least 5, 5 or fewer, at least 6, 6 or fewer, at least 7, 7 or fewer, at least 8, 8 or fewer, at least 9, 9 or fewer, at least 10, 10 or fewer, or greater than 10 amino acid substitutions in the resulting encoded polypeptide. In some instances, a useful sequence, including amino acid and nucleic acid sequences such as but not limited to those such sequences described herein, may share 100% sequence identity with a sequence described herein. In some instances, a useful sequence, including amino acid and nucleic acid sequences such as but not limited to those such sequences described herein, may share less than 100% sequence identity with a sequence described herein, including e.g., at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 65%, at least 60%, at least 55%, at least 50%, at least 45%, at least 40%, at least 35%, or at least 30% sequence identity with a sequence, including but not limited to e.g., an amino acid or nucleic acid sequence described herein.

Useful nucleic acids encoding a gene product present on a transgene will vary and may provide for various functions, including e.g., correction of a defective gene in the host cell or organism, encoding and/or expression of a heterologous gene product in the cell, encoding and/or expression of one or more additional copies of an endogenous gene product in the cell, inhibition of the expression of a gene or a gene product in the cell, or the like. Useful nucleic acids include but are not limited to e.g., expression cassettes, recombinant mRNA, recombinant vector genomes (such as e.g., recombinant viral genomes), recombinant plasmids, minicircle plasmids, minigenes, microgenes, artificial chromosomes, interfering nucleic acids (e.g., siRNA, shRNA, etc.), and the like.

Useful gene products, e.g., of a functionally integrated transgene, include but are not limited to e.g., noncoding nucleic acids and nucleic acids coding for one or more proteins and/or peptides. In some embodiments, a gene product of a transgene or a coding region of a vector may include nucleic acid sequence coding for an enzyme, such as e.g., a nuclease, a DNA base editor, an RNA editor, or the like. In some embodiments, a sequence encoding a gene product may include, alone or with other payload elements, a noncoding nucleic acid such as e.g., a microRNA (i.e., miRNA), shRNA, siRNA, piRNA, snoRNA, snRNA, exRNA, scaRNA, lncRNA, guide RNA (gRNA, sgRNA, etc.), or the like.

In some instances, cell populations that include hepatocytes and/or hepatocyte progenitors may be edited at a target locus. Essentially any locus may be targeted including but not limited e.g., loci that include liver-associated genes and/or regulatory elements thereof, loci that include immune related genes and/or regulatory elements thereof, and the like. The edit introduced at a target locus may vary where useful edits include but are not limited to e.g., a deletion, an insertion, a substitution, a frameshift, and the like.

Non-limiting examples of useful deletions include: deletion of 1, 2, 3, 4, 5, 6, 7, 8, 9, 10, or more bases; deletion of 1 or more, 2 or more, or 3 or more functional domains, and/or portions thereof, of a gene; deletion of 1 or more, 2 or more, or 3 or more exons, and/or portions thereof, deletion of all, all except 1, all except 2, or all except 3 exons, and/or portions thereof, of a gene; deletion of a regulatory element (e.g., promoter, enhancer, etc.) of a gene; and the like. Non-limiting examples of useful insertions include: insertion of 1, 2, 3, 4, 5, 6, 7, 8, 9, 10, or more bases; insertion of 1 or more, 2 or more, or 3 or more functional domains, and/or portions thereof, of a gene; insertion of 1 or more, 2 or more, or 3 or more exons, and/or portions thereof, insertion of all, all except 1, all except 2, or all except 3 exons, and/or portions thereof, of a gene; insertion of a regulatory element (e.g., promoter, enhancer, etc.) of a gene; and the like. The size of introduced deletions and/or insertions will vary and may range from 1 base to 500 bases or more, including but not limited to e.g., 1 to 400, 1 to 350, 1 to 300, 1 to 250, 1 to 200, 1 to 150, 1 to 100, 1 to 50, 10 to 400, 10 to 350, 10 to 300, 10 to 250, 10 to 200, 10 to 150, 10 to 100, 10 to 50, 25 to 400, 25 to 350, 25 to 300, 25 to 250, 25 to 200, 25 to 150, 25 to 100, 25 to 50, 50 to 400, 50 to 350, 50 to 300, 50 to 250, 50 to 200, 50 to 150, 50 to 100, 100 to 400, 100 to 350, 100 to 300, 100 to 250, 100 to 200, 100 to 500, 200 to 500, 300 to 500, 400 to 500, at least 1, at least 2, at least 10, at least 25, at least 50, at least 75, at least 100, at least 150, at least 200, 500 or less, 400 or less, 350 or less, 300 or less, 250 or less, 200 or less, 150 or less, 100 or less, or 50 or less bases.

Non-limiting examples of useful substitutions include: substitutions introducing a premature stop codon; substitutions ablating a stop codon; substitutions resulting in an amino acid change; and the like. One or multiple substitutions at the nucleic acid level (e.g., substitution of 1, 2, or 3 bases) may be employed to introduce essentially any amino acid to amino acid substitution at the polypeptide level as desired.

In some instances, useful edits may ablate or delete all or a portion of an endogenous gene or otherwise render non-functional one or more endogenous genes, such as but not limited to e.g., one or more immune-related genes, or the encoded product of such a gene, such as an immune-related protein. Such deletion of a gene, or portion thereof, rendering the gene and/or the encoded product non-functional may be referred to as a knock-out. In some instances, a gene, or the gene product thereof, may be rendered non-functional through introduction of an insertion, e.g., causing a frameshift or generating a misfolded or otherwise non-functional protein.

In some instances, useful edits may correct a dysfunctional gene, including e.g., a dysfunctional gene of a monogenic disease. In some instances, the monogenic disease is a liver-associated monogenic disease (i.e., a monogenetic disease arising from a dysfunctional gene that is liver-associated or is a hepatocyte-associated gene). In some instances, the monogenic disease is a non-liver-associated monogenic disease (i.e., a monogenetic disease arising from a dysfunctional gene that is not a liver-associated or hepatocyte-associated gene). In some instances, the edit is a corrective edit of a defective endogenous locus. In some instances, the edit is not a corrective edit of a defective endogenous locus.

In some instances, an edit may be introduced into a non-hepatocyte and/or non-liver associated locus such that the edit is in a locus that is not associated with hepatocyte and/or liver function. By “locus associated with hepatocyte function” or “hepatocyte locus” or similar terms, as used herein, is meant that the locus includes a coding (e.g., exon) or non-coding regulator region (e.g., intron, promoter, enhancer, etc.) of a gene associated with hepatocyte function and/or a function primarily carried out in the liver, such as e.g., liver metabolism (e.g., hepatocyte metabolism), ammonia metabolism (inc. e.g., urea cycle), amino acid metabolism, amino acid bio-synthesis, amino acid catabolism, detoxification, synthesis of liver (e.g., hepatocyte) proteins (including e.g., albumin, fibrinogen, prothrombin, clotting factor (e.g., factor V, VII, IX, X, XI, and XII), protein C, protein S, antithrombin, lipoprotein, ceruloplasmin, transferrin, complement proteins, proteins of the hepatocyte proteome and/or secretome (such as e.g., those described in Franko et al. Nutrients. (2019) 11 (8): 1795; the disclosure of which is incorporated herein by reference in its entirety)), and the like.

In some instances, multiple gene edits may be introduced into a single cell. For example, in some instances, a cell may include more than one deletion, insertion, substitution, or some combination thereof, including e.g., where the cell include 2, 3, 4, or 5 such edits. Any useful combination of edits may be introduced including e.g., multiple edits in a single gene, edits in two or more polypeptides or chains of a single protein, edits in two or more different proteins of a family or pathway, edits in two or more functionally-related proteins (e.g., two or more immune-related proteins, two or more liver-associated proteins, etc.), and the like.

Where multiple edits are introduced, and/or a cell is genetically modified in multiple ways, such as e.g., through introduction of an edit at an endogenous locus and functional integration of a transgene, or introduction of multiple edits at multiple endogenous loci, or a combination thereof; such multiple edits/modifications may be performed simultaneously and/or in any convenient and appropriate order. For example, in some instances, a cell population may be contacted simultaneously, or essentially simultaneously, with reagents to make two different genetic modifications. In some instances, a cell population will be contacted with a first reagent, or set of reagents, to make a first modification and subsequently contacted with a second reagent, or set of reagents, to make a second modification. In some instances, where steps are performed sequentially, one or more intervening actions may be performed, including but not limited to e.g., isolation, purification, enrichment, cell culture, expansion, analysis, cryopreservation, and/or the like. In some instances, no intervening actions, such as e.g., isolation, purification, enrichment, cell culture, expansion, analysis, cryopreservation, and/or the like, are performed.

Various convenient methods of contacting a cell population with one or more editing reagents may be employed including but not limited to e.g., transfection of editing reagents or nucleic acids encoding such agents, transduction of editing reagents, nucleofection and/or electroporation of editing reagents, and the like. In some instances, a vector, e.g., a viral vector or a non-viral vector may be employed. In some instances, the components of the vector may include nucleic acids, proteins, or a combination thereof. Any convenient viral or non-viral vector may be employed including but not limited to e.g., lipid nanoparticle (LNP) vectors.

Vectors may be configured to contain all, or less than all, of the components necessary for performing a desired edit. For example, in some instances, a vector may include all components sufficient for performing an edit at a targeted locus. In some instances, a vector may include less than all of the components needed for performing an edit and the remaining components may be delivered by other means, e.g., another different vector, transduction, transfection, or the like. In some instances, components, e.g., nucleic acid and protein components, of a targeting system may be pre-complexed prior to delivery, including where such components are pre-complexed within a delivery vector. For example, in some instances nucleic acid (e.g., a gRNA, etc.) and protein (e.g., nuclease(s) or base editing protein(s), etc.) editing reagents of an editing system may be complexed as ribonucleoprotein (RNP) for delivery to a cell population for editing.

Any convenient and appropriate gene editing system may be employed to introduce one or more of the edits described herein. Methods of site-directed introduction of a desired edit will vary and may include introducing one or more site directed cleavage events, e.g., through the use of one or more site-directed nucleases (e.g., a CRISPR/Cas9 nuclease, a TALEN nuclease, a ZFN, and the like). Site-directed cleavage may include double and/or single strand breaks where applicable. In some instances, site-directed cleavage is followed by a specific repair event at the site cleaved by the site-directed nuclease, e.g., to introduce a desired edit, such as e.g., a substitution, insertion, deletion, or the like. Such methods of specific repair may include, e.g., homologous recombination, including homology directed repair (HDR), e.g., in the presence of a nucleic acid that includes homology regions to guide the repair. In some instances, site-directed cleavage may be employed to introduce a gene disruption and/or knock-out, e.g., without employing a specific repair event, e.g., through cellular processes following site-directed cleavage such as e.g., non-homologous end joining (NHEJ). In some instances, site-directed introduction of a desired edit may employ a base editing system that does not introduce a double strand cleavage event, such as but not limited to e.g., CRISPR protein-guided based editing systems, such as e.g., dCas9-deaminase fusion protein systems including cytosine base editor (CBE) and adenine base editor (ABE) systems. In some instances, useful base editing systems introduce a single base change, e.g., without cleavage of the phosphodiester nucleic acid backbone.

Various editing compositions may be employed and such compositions will vary, e.g., based on the editing-system employed, the type of edit desired, the sequence of the targeted locus or loci, etc. Useful editing compositions may include e.g., CRISPR/Cas9 editing compositions, e.g., including a Cas9 protein, or a nucleic acid encoding a Cas9 protein, and gRNAs or a sgRNA or a nucleic acid encoding the gRNAs or sgRNA; TALEN editing compositions, including e.g., a TALEN nuclease or TALEN nuclease pair, or a nucleic acid encoding a TALEN nuclease or TALEN nuclease pair; ZFN editing compositions, including e.g., a ZFN nuclease or ZFN nuclease pair, or a nucleic acid encoding a ZFN nuclease or ZEN nuclease pair; base-editing editing compositions e.g., including a CRISPR-protein-guided-base-editing protein, or a nucleic acid encoding a CRISPR-protein-guided-base-editing protein, and gRNAs or a sgRNA or a nucleic acid encoding the gRNAs or sgRNA; and the like.

CRISPR-Cas based editing compositions, and methods of employing CRISPR-Cas based editing compositions will be described in more detail. However, such description, as well as the compositions and methods, are not so limited and it will be readily understood that elements, targets, and/or concepts of such description may be correspondingly adapted or applied to the use of other editing systems where appropriate.

In some instances, useful editing compositions will include a CRISPR-Cas protein, such as e.g., a Cas9 protein, or a polynucleotide encoding a CRISPR-Cas protein and guide RNA (gRNA) or a polynucleotide encoding gRNA. As used herein, the term “gRNA” generally encompasses either two-component guide systems (e.g., two gRNAs) as well as single guide RNA (sgRNA) systems, unless inappropriate and/or denoted otherwise. In some instances, the gRNA or multiple gRNAs may be configured and employed to target a desired locus as described herein or one or more elements thereof such as one of more exons of a gene present at the locus. For example, in some instances, a gRNA or multiple gRNAs may be configured and employed to target a B2M locus or one or more elements thereof, such as e.g., one or more exons (e.g., one or more of exon 1, exon 2, or exon 3) of a B2M locus.

In some instances, an instant method of editing may include the use of a Cas9 nuclease, including natural and engineered Cas9 nucleases, as well as nucleic acid sequences encoding the same. Useful Cas9 nucleases include but are not limited to e.g., Streptococcus pyogenes Cas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1: e60 and Chylinski et al. (2013) RNA Biology 10:726-737, the disclosures of which are incorporated herein by reference in their entirety. In some instances, a non-Cas9 CRISPR nuclease (or engineered variant thereof) may be employed, including but not limited to e.g., Cpf1 or Cpf1 variant.

Cas9 nucleases are used in the CRISPR/Cas9 system of gene editing and modified Cas9 proteins (e.g., Cas9 nickases and dCas9 proteins, with or without added functionalities) may be employed in various editing methodologies. In the CRISPR/Cas9 system two separate guide components (i.e., crRNA and a tracrRNA) or a chimeric RNA containing the target sequence (i.e., the “guide RNA” or “single guide RNA (sgRNA)”, which collectively contains a crRNA and a tracrRNA) guides the Cas9 nuclease to cleave the DNA at a specific target sequence defined by the gRNAs or sgRNA. Where employed, the specificity, efficiency and versatility of targeting and replacement by HDR is greatly improved through the combined use of various homology-directed repair strategies and CRISPR nucleases (see e.g., Gratz et al. (2014) Genetics. 196 (4) 961-971; Chu et al. (2015) Nature. 33:543-548; Hisano et al. (2015) Scientific Reports 5:8841; Farboud & Meyer (2015) Genetics, 199:959-971; Merkert & Martin (2016) Stem Cell Research 16 (2): 377-386; the disclosures of which are incorporated herein by reference in their entirety).

The CRISPR system offers significant versatility in gene editing in part because of the small size and high frequency of necessary sequence targeting elements within host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target nucleic acids. The PAM sequence directly downstream of the target sequence is not part of the guide RNA but is obligatory for cutting the DNA strand. Synthetic Cas9 nucleases have been generated with novel PAM recognition, further increasing the versatility of targeting, and may be used in the methods described herein. Cas9 nickases (e.g., Cas9 (D10A) and the like) that cleave only one strand of target nucleic acid as well as endonuclease deficient (i.e., “dead”) dCas9 variants with additional enzymatic activities added by an attached fusion protein have also been developed.

In some embodiments, immune-related loci of hepatocytes and/or hepatocyte progenitors may be targeted for editing, e.g., to render the edited hepatocytes and/or hepatocyte progenitors hypoimmunogenic. For example, in some instances, one or more loci encoding HLA class I proteins or related proteins (e.g., HLA class 1a or related proteins), one or more loci encoding HLA class II proteins or related proteins (e.g., “HLA-D” proteins, transcription factors and/or coactivators that causes expression of an HLA class II genes, or the like), or combinations thereof may be targeted. By introducing such disruptions in HLA class I and/or HLA class II proteins hepatocytes and/or hepatocyte progenitors, as shown herein, may be rendered hypoimmunogenic. For example, such disruptions may result in reduced killing of the edited hepatocytes by immune cells, such as e.g., lymphocytes such as e.g., cytotoxic T lymphocytes (CTLs). Useful loci for targeting include but are not limited to e.g., HLA-A, HLA-B, HLA-C, HLA-DP, HLA-DM, HLA-DOA, HLA-DOB, HLA-DQ, HLA-DR, B2M, CIITA, NLRC5, RFX5, RFXANK, RFXAP, and the like. In some instances, a desired edit may disrupt all genes of a particular class, e.g., by introducing one or more edits resulting in e.g., HLA-A, -B, and -C disruption and/or introducing one or more edits of a component shared by HLA-A, -B, and -C such as e.g., B2M. Similar strategies may be adapted and employed for other targets and loci, such as e.g., HLA class II proteins. Useful HLA genes, alleles, loci, and the proteins thereof include e.g., those described in Marsh et al. (2010) Tissue Antigens 75:291-455; the disclosure of which is incorporated herein by reference in its entirety.

In some instances, useful CRISPR Cas9-based B2M targeting sequences and corresponding PAM sequences that may be employed for editing a B2M locus as described herein include e.g.: the B2M exon 1 targeting sequence “B2M_Ex1_7” having the sequence GGCCACGGAGCGAGACATCT (SEQ ID NO:039) (PAM, CGG); the B2M exon 1 targeting sequence “B2M_Ex1_3” having the sequence CGCGAGCACAGCTAAGGCCA (SEQ ID NO: 040) (PAM, CGG); the B2M exon 2 targeting sequence “B2M_Ex2_4” having the sequence AAGTCAACTTCAATGTCGGA (SEQ ID NO:041) (PAM, TGG); and the like.

In some instances, an editing composition may be an HLA class I-targeting composition and/or a HLA class II-targeting composition, resulting in a disruption in the production and/or function of one or more HLA class I proteins, one or more HLA class II proteins, and/or one or more associated proteins such as but not limited to e.g., B2M, a transcription factor that causes expression of an HLA class I and/or II gene or protein, and/or a coactivator that causes expression of an HLA class I and/or II gene or protein.

Such editing compositions may be contacted with a cell population under conditions sufficient to generate the desired edit, including e.g., where such conditions are sufficient for the introduction, delivery, transfection, transduction, targeting, enzymatic activity, and/or repair (where applicable), as well as survival and necessary biological activities of the cells. Conditions sufficient to generate desired edits may include but are not limited to e.g., suitable culture conditions, including e.g., maintenance at a suitable environmental conditions (e.g., temperature, gas exchange, etc.) in a suitable culture medium conducive to the editing reaction, and the like. In addition, editing reactions may be carried out for a sufficient amount of time for the editing reaction to take place and reach desired levels of completion, where such sufficient amounts of time will vary. Also, in some instances, the time of exposure to editing reagents may be minimized, e.g., where an editing reaction or components thereof may have one or more detrimental effects on a cell population, such as e.g., decreased cell viability, increased cell fragility, and the like.

In some instances, a method of gene editing may include the use of a zinc-finger nuclease (ZFN). ZFNs consist of the sequence-independent Fokl nuclease domain fused to zinc finger proteins (ZFPs). ZFPs can be altered to change their sequence specificity. Cleavage of targeted dsDNA involves binding of two ZFNs (designated left and right) to adjacent half-sites on opposite strands with correct orientation and spacing, thus forming a Fokl dimer. Dimerization increases ZFN specificity significantly. Three or four finger ZFPs target about 9 or 12 bases per ZFN, or about 18 or 24 bases for the ZFN pair. The specificity, efficiency and versatility of targeting and replacement of homologous recombination is greatly improved through the combined use of various homology-directed repair strategies and ZFNs (see e.g., Urnov et al. (2005) Nature. 435 (7042): 646-5; Beumer et al (2006) Genetics. 172 (4): 2391-2403; Meng et al (2008) Nat Biotechnol. 26 (6): 695-701; Perez et al. (2008) Nat Biotechnol. 26 (7): 808-816; Hockemeyer et al. (2009) Nat Biotechnol. 27 (9): 851-7; the disclosures of which are incorporated herein by reference in their entirety). In general, one ZFN site can be found every 125-500 bp of a random genomic sequence, depending on the assembly method. Methods for identifying appropriate ZFN targeting sites include computer-mediated methods e.g., as described in e.g., Cradick et al. (2011) BMC Bioinformatics. 12:152, the disclosure of which is incorporated herein by reference in its entirety.

In some instances, a method of gene editing may include the use of a transcription activator-like effector nuclease (TALEN). Similar in principle to the ZEN nucleases, TALENs utilize the sequence-independent Fokl nuclease domain fused to Transcription activator-like effectors (TALEs) proteins that, unlike ZNF, individually recognize single nucleotides. TALEs generally contain a characteristic central domain of DNA-binding tandem repeats, a nuclear localization signal, and a C-terminal transcriptional activation domain. A typical repeat is 33-35 amino acids in length and contains two hypervariable amino acid residues at positions 12 and 13, known as the “repeat variable di-residue” (RVD). An RVD is able to recognize one specific DNA base pair and sequential repeats match consecutive DNA sequences. Target DNA specificity is based on the simple code of the RVDs, which thus enables prediction of target DNA sequences. Native TALEs or engineered/modified TALEs may be used in TALENs, depending on the desired targeting. TALENs can be designed for almost any sequence stretch. Merely the presence of a thymine at each 5′ end of the DNA recognition site is required. The specificity, efficiency and versatility of targeting and replacement of homologous recombination is greatly improved through the combined use of various homology-directed repair strategies and TALENs (see e.g., Zu et al. (2013) Nature Methods. 10:329-331; Cui et al. (2015) Scientific Reports 5:10482; Liu et al. (2012) J. Genet. Genomics. 39:209-215, Bedell et al. (2012) Nature. 491:114-118, Wang et al. (2013) Nat. Biotechnol. 31:530-532; Ding et al. (2013) Cell Stem Cell. 12:238-251; Wefers et al. (2013) Proc. Natl. Acad. Sci. U.S.A, 110:3782-3787; the disclosures of which are incorporated herein by reference in their entirety).

In some instances, a method of gene editing may include the use of a base editor system, including but not limited to e.g., base editor systems employing a fusion protein comprising a programmable DNA binding protein, a nucleobase editor and gRNA, and the like. Base editing will generally not rely on HDR and/or NHEJ and will generally not result in or require the cleavage of phosphodiester bonds on both backbones of dsDNA. Thus, based editing may, in some instances, employ RNA-guided (i.e., “programmable”) DNA binding proteins, such as Cas nucleases, that do not cause double-strand breaks, such as e.g., nuclease-deficient or nuclease-defective Cas proteins, such as e.g., a dCas9 or a Cas9 nickase. Useful examples of base editors and base editing systems, including base editor encoding nucleic acids, include but are not limited to BE1, BE2, BE3 (Komor et al., 2016); Target-AID (Nishida et al., 2016); SaBE3, BE3 PAM variants, BE3 editing window variants (Kim et al., 2017); HF-BE3 (Rees et al., 2017); BE4 and BE4-Gam; AID, CDA1 and APOBEC3G BE3 variants (Komor et al., 2017); BE4max, ArcBe4max, ABEmax (Koblan et al., 2018); Adenine base editors (ABE7.10) (Gaudelli et al., 2017); ABE8 (Richter et al., 2020); ABE8e (Gaudelli et al., 2020); A&C-BEmax (Zhang et al., 2020); SPACE (Grünewald et al., 2020); and the like; the preceding references being incorporated by reference herein in their entirety.

Non-limiting examples of useful base editor systems, and the components thereof, include but are not limited to e.g., those describe in PCT Patent App. Pub. Nos. WO2020236936A1, WO2020231863A1, WO2020168135A1, WO2020168122A1, WO2020168088A1, WO2020168132A9, WO2020168133A1, WO2020168075A9, WO2020168051A9, WO2020160514A1, WO2020160517A1, WO2020150534A9, WO2020051562A3, WO2020051562A3, WO2020028823A1, WO2019217941A1, WO2019217942A1, and WO2019217943A1; as well as US. Pat. App. Pub. No. US20200399626A1; the disclosures of which are incorporated herein by reference in their entirety.

In some instances, the presence of a desired edit may be verified, e.g., by an assay to test whether the edit is present (or the desired deletion is absent) at the target locus, by an assay to test whether the gene product encoded at the locus targeted for disruption is absent, that the gene product encoded by an introduced sequence is present, or the like. Useful methods to perform such assays include but are not limited to e.g., methods based on PCR (e.g., PCR, qPCR, rt-PCR, etc.) of the locus and/or a RNA encoded at the locus, methods based on Western blot of cells lysates probed with antibodies to a protein encoded at the locus, flow cytometry based methods, sequencing (e.g., single cell sequencing), and the like.

Other useful components, e.g., of transgenes, of expression cassettes, of editing compositions, of vectors, or the like, may include promoter sequences (e.g., constitutive, tissue-specific, etc.), signal peptide sequences, poly (A) sequences, terminators, translational regulatory sequences such as ribosome binding sites and internal ribosome entry sites, enhancers, silencers, insulators, boundary elements, replication origins, matrix attachment sites and/or locus control regions. Furthermore, multiple gene products can be expressed from one nucleic acid, for example by linking individual components (transgenes) in one open reading frame separated, for example, by a self-cleaving 2A peptide or IRES sequence.

Examples of useful promoters include, for example, viral simian virus 40 (SV40) (e.g., early or late), cytomegalovirus (CMV) (e.g., immediate early), Moloney murine leukemia virus (MoMLV), MND (myeloproliferative sarcoma virus enhancer, negative control region deleted, d1587rev primer-binding site substituted), Rous sarcoma virus (RSV), herpes simplex virus (HSV), spleen focus-forming virus (SFFV) promoters and the like. In certain embodiments, the promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured. In some instances, the promoter may be constitutive. In some instances, the promoter may cause preferential expression in a desired cell-type or tissue, e.g., the promoter may be cell-type or tissue specific.

In some instances, a transgene, an expression cassette, a vector, etc., may include sequence encoding a signal peptide. Signal peptides are short peptides located in the N-terminal of proteins. Functioning in protein localization, signal peptides are useful in directing the associated protein to the secretory pathway and driving secretion of the protein.

Vectors, including retroviral vectors, e.g., lentivirus vectors, may include (or exclude as desired where appropriate) various elements, including cis-acting elements, such as promoters, long terminal repeats (LTR), and/or elements thereof, including 5′ LTRs and 3′ LTRs and elements thereof, central polypurine tract (cPPT) elements, DNA flap (FLAP) elements, export elements (e.g., rev response element (RRE), hepatitis B virus posttranscriptional regulatory element (HPRE), etc.), posttranscriptional regulatory elements (e.g., woodchuck hepatitis virus posttranscriptional regulatory element (WPRE), hepatitis B virus regulatory element (HPRE), etc.), polyadenylation sites, transcription termination signals, insulators elements (e.g., β-globin insulator, e.g., chicken HS4), and the like. Other elements that may be present or absent in various vectors include but are not limited to enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, homology regions useful in homology directed repair (HDR), and the like.

Useful LTRs include but are not limited to e.g., those containing U3, R and/or U5 regions, and portions thereof. LTRs provide functions for the expression of retroviral genes (e.g., promotion, initiation and polyadenylation of gene transcripts) and for viral replication. An LTR can contain numerous regulatory signals including transcriptional control elements, polyadenylation signals and sequences needed for replication and integration of the viral genome. A U3 region may contain enhancer and promoter elements. A U5 region may contain a polyadenylation sequence. The R (repeat) region is generally flanked by the U3 and U5 regions. An LTR composed of U3, R and U5 regions may appear at both the 5′ and 3′ ends of a viral genome. A viral genome may include sequence adjacent to a 5′ LTR that functions in reverse transcription of the genome (e.g., the tRNA primer binding site), for efficient packaging of viral RNA into particles (e.g., the Psi site), and the like.

Useful LTRs include modified 5′ LTR and/or 3′ LTRs. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

In some embodiments, useful vectors may be self-inactivating. The term “self-inactivating” (SIN) with regards to vectors refers to replication-defective vectors, e.g., retroviral or lentiviral vectors, in which the right (3′) LTR enhancer-promoter region, including e.g., the U3 region, has been modified (e.g., by deletion and/or substitution) to prevent viral transcription beyond the first round of viral replication. In further embodiments, the 3′ LTR may be modified such that the U5 region is replaced, for example, with a heterologous or synthetic poly (A) sequence, one or more insulator elements, and/or an inducible promoter. It will be readily apparent to the ordinarily skilled artisan where reference to an LTR, e.g., 3′ LTR or 5′ LTR, may include modified LTRs or modifications to LTRs, such as modifications to the 3′ LTR, the 5′ LTR, or both 3′ and 5′ LTRs.

In some embodiments, viral vectors may comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. In some embodiments, a vector may not include a TAR element, including e.g., wherein the U3 region of the 5′ LTR is replaced by a heterologous promoter.

In some instances, a vector may be a pseudotyped vector. The terms “pseudotype” or “pseudotyping” as used herein, refer to a virus that has one or more viral envelope proteins that have been substituted with those of another virus possessing preferable characteristics. For example, HIV can be pseudotyped with vesicular stomatitis virus G-protein (VSV-G) envelope proteins. In some embodiments, lentiviral envelope proteins are pseudotyped with VSV-G. In some embodiments, packaging cells which produce recombinant retrovirus, e.g., lentivirus, pseudotyped with the VSV-G envelope glycoprotein may be employed.

Vectors, both viral and nonviral, may include structural and/or genetic elements, or portions thereof, derived from viruses. Retroviral vectors may include structural and/or genetic elements, or portions thereof, derived from retroviruses. Lentiviral vectors may include structural and functional genetic elements, or portions thereof, including LTRs that are primarily derived from a lentivirus. In some instances, hybrid vectors may be employed, including e.g., where a hybrid vector includes an LTR or other nucleic acid containing both retroviral, e.g., lentiviral, sequences and non-retroviral, e.g., non-lentiviral viral, sequences. In some embodiments, a hybrid vector may include a vector comprising retroviral e.g., lentiviral, sequences for reverse transcription, replication, integration and/or packaging.

The cell populations, and/or hepatocytes and/or hepatocyte progenitors thereof, can be used for the treatment of a subject for a condition where administration of an effective amount of the cells will have a desired therapeutic effect. In some instances, the desired therapeutic effect will be a result of one or more endogenous functions of the administered hepatocytes (e.g., endogenous function(s) of healthy hepatocytes, endogenous hepatocyte function(s) of hypoimmunogenic hepatocytes, and the like), including but not limited to e.g., hepatocyte metabolism, detoxification, synthesis of hepatocyte proteins (including e.g., albumin, fibrinogen, prothrombin, clotting factor (e.g., factor V, VII, IX, X, XI, and XII), protein C, protein S, antithrombin, lipoprotein, ceruloplasmin, transferrin, complement proteins, proteins of the hepatocyte proteome and/or secretome (such as e.g., those described in Franko et al. Nutrients. (2019) 11 (8): 1795; the disclosure of which is incorporated herein by reference in its entirety)), and the like. In some instances, the desired therapeutic effect will be a result of one or more heterologous functions of the administered hepatocytes, e.g., a heterologous function of a gene product encoded by a functionally integrated transgene. In some instances, when the condition of the subject is hemophilia (e.g., Hemophilia A or Hemophilia B) and the methods include administering to the subject an effective amount of genetically modified human hepatocytes comprising a transgene encoding a gene product for treating the hemophilia (e.g., Factor VIII, Factor IX, and/or the like), the methods may further include modulating coagulation in the subject, e.g., by administration of an anti-coagulant (e.g., warfarin, rivaroxaban, dabigatran, apixaban, edoxaban, and/or the like) to the subject in an amount effective to modulate coagulation in the subject.

Cell populations including hepatocytes and/or progenitors thereof as described herein can be used for treatment and/or prevention of any liver disease or disorder. For example, reconstitution of liver tissue in a patient by the introduction of hepatocytes is a potential therapeutic option for patients with any liver condition(s) (e.g., acute liver failure, chronic liver disease and/or metabolic or monogenic disease), including as a permanent treatment for these conditions by repopulating the subject's liver with genetically modified cells as described herein. Hepatocyte reconstitution may be used, for example, to introduce genetically modified hepatocytes for gene therapy or to replace hepatocytes lost as a result of disease, physical or chemical injury, or malignancy. In addition, expanded human hepatocytes can be used to populate artificial liver assist devices.

Disclosed herein are methods of producing, including expanding, hepatocytes for various purposes. In some instances, the instant methods provide for the production and/or expansion of human hepatocytes suitable for transplantation into a subject in need thereof, including human hepatocytes suitable for transplantation, including e.g., orthotopic liver transplantation. Hepatocytes, including human hepatocytes, produced according to the methods described herein can be purified, cryopreserved, and/or extensively characterized prior to transplantation or infusion. Among other uses, hepatocytes produced according to the methods described herein may provide on-demand therapy for patients with one or more severe liver diseases.

In some instances, the desired therapeutic effect will be a result of one or more heterologous functions of the administered hepatocytes conferred by a gene product encoded by an integrated transgene. Accordingly, essentially any condition that may be treated through delivery of a heterologous gene product, such as a secreted heterologous gene product, may be treated using genetically modified hepatocytes generated as described herein. For example, a monogenic disease resulting in a deficiency of a protein may be treated through administration of an effective amount of hepatocytes genetically modified to contain an integrated transgene encoding the protein, thereby reducing the deficiency of the protein.

Useful transgene for treating monogenic conditions include, but are not limited to e.g., transgenes encoding full-length and modified forms of Copper-transporting ATPase 2 (ATP7B), Hereditary hemochromatosis protein (HFE), Hemojuvelin, Hepcidin (HAMP), Transferrin receptor protein 2 (TFR2), Solute carrier family 40 member 1 (SLC40A1), Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase (ARG, including ARG1), alpha-1 antitrypsin (AAT), fumarylacetoacetate hydrolase (FAH), Argininosuccinate lyase (ASL), Argininosuccinate synthase (ASS, including ASS1), Ornithine translocase (ORNT1), citrin, UDP-glucuronosyltransferase 1A1 (UGT1A1), Transthyretin (TTR), Serine—pyruvate aminotransferase (AGXT), Complement factor H (CFH), the like, and combinations thereof.

In some instances, the treated disease is a liver disease and/or a liver-associated monogenic disease, including e.g., where the gene product of the transgene is a liver-associated protein. In some instances, the treated disease is a liver disease and/or a liver-associated monogenic disease, including e.g., where the gene product of the transgene is not a liver-associated protein. In some instances, the treated monogenic disease is not a liver-associated monogenic disease, including e.g., where the gene product of the integrated transgene is not a liver-associated protein. In some instances, the treated disease is not a liver disease, including e.g., where the gene product of the integrated transgene is not a liver-associated protein.

Cell populations including hepatocytes and/or hepatocyte progenitors as described herein and compositions comprising such cells as described herein can be administered to subjects by any suitable means and to any part, organ, or tissue of the subject. Non-limiting examples of administration means include portal vein infusion, umbilical vein infusion, direct splenic capsule injection, splenic artery infusion, infusion into the omental bursa and/or intraperitoneal injection (infusion, transplantation). In certain embodiments, the compositions comprise encapsulated hepatocytes that are transplanted by infusion into the intraperitoneal space and/or the omental bursa. In certain embodiments, the compositions comprise acellular/decellularized scaffold, including e.g., synthetic scaffolds, decellularized liver, and the like, that are seeded and/or repopulated with hepatocytes as described herein and surgically transplanted into a subject in need thereof.

In addition to or as an alternative to administration (transplantation) to a subject (patient), the hepatocytes as described herein can also be used for supplying hepatocytes to devices or compositions useful in treating subjects with liver disease. Non-limiting examples of such devices or compositions in which the hepatocytes of the present disclosure can be used include bioartificial livers (BAL) (extracorporeal supportive devices for subjects suffering from acute liver failure) and/or decellularized livers (recellularizing organ scaffolds to provide liver function in the subject). See, e.g., Shaheen et al. (2019) Nat Biomed Eng. doi: 10.1038/s41551-019-0460-x; Glorioso et al. (2015) J Hepatol 63 (2): 388-98.

In some instances, a subject receiving a treatment as described herein may not receive immunosuppressants, e.g., the subject may be non-immunosuppressed and/or immunologically normal at the time of therapy, e.g., before, during, and/or after administration of hepatocytes as described herein. For example, such a subject may have one or more contraindications to immunosuppression, immunosuppressants, and/or a particular immunosuppressive therapy, or may not be administered an immunosuppressant for different reason. In some instances, a non-immunosuppressed subject and/or a subject with a contraindication to immunosuppression may be administered a cell population of which a substantial portion, including all or essentially all, of the population is hypoimmunogenic hepatocytes.

Non-limiting examples of contraindications to immunosuppression include: liver disease or condition, fibrosis, cirrhosis, kidney disease or condition, a blood disease or condition, history of shingles and/or chickenpox, infection (e.g., tuberculosis, BK polyomavirus, herpes simplex, fungus, parasite (e.g., roundworm Strongyloides), varicella zoster virus, measles, etc.), exposure to infectious agent (e.g., measles, chickenpox, etc.), cancer, malignancy, diabetes, high cholesterol, high blood pressure, high blood triglycerides, hemolytic uremic syndrome, anemia, decreased blood platelets, low WBC count, pericardial effusion, thrombotic thrombocytopenia purpura, pulmonary edema, interstitial pneumonitis, stomatitis, stomatitis, acute kidney failure, renal artery occlusion (e.g., renal artery thrombosis), visible edema, ascites, proteinuria, impaired wound healing, pregnancy, lactation and breastfeeding, malignant lymphoma, thrombosis (e.g., post-liver transplant thrombosis), heart transplant, endocrine disorder of hormone deficiency (e.g., thyroid hormone deficiency, hypothalamus insufficiency, pituitary insufficiency), low blood potassium, psychotic disorder, myopathy, glaucoma, cataracts, ulcers, gastritis, diverticulitis, intestinal anastomosis, tendon rupture, osteoporosis, low bone calcification or density, seizures, argininosuccinate lyase deficiency, carbamoyl phosphate synthetase deficiency, citrullinemia, ornithine carbamoyltransferase deficiency, arginase deficiency, elevated creatine kinase, broken bone due to disease or illness, osteonecrosis, muscle wasting, hyperammonemia (e.g., as associated with N-acetylglutamate synthase deficiency), allergy to immunosuppressants (such as e.g., corticosteroids (e.g., prednisone, budesonide, prednisolone), janus kinase inhibitors (e.g., tofacitinib), calcineurin inhibitors (e.g., cyclosporine, tacrolimus), mTOR inhibitors (e.g., sirolimus, everolimus), IMDH inhibitors (e.g., azathioprine, leflunomide, mycophenolate, immunosuppressant biologics (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)), etc.), and the like. Other contraindications, e.g., for specific immunosuppressants, are readily ascertainable from the label information, drug and drug interaction databases, drug manufacturer, and/or relevant regulatory agency such as e.g., the U.S. Food and Drug Administration (FDA).

In some instances, an administered cell population may be 80% or greater hypoimmunogenic hepatocytes, including e.g., 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, or 99% or greater hypoimmunogenic hepatocytes. In some instances, a subject with one or more contraindications to treatment with one or more immunosuppressants may be administered a cell population having 80% or greater hypoimmunogenic hepatocytes, including e.g., where such hypoimmunogenic hepatocytes include one or more, including two or more, including at least three genetic modifications as described herein.

Disease and disorders, including in subjects with or without one or more contraindications to immunosuppression, that can be treated using the methods and/or cell populations described herein include but are not limited to Crigler-Najjar syndrome type 1; familial hypercholesterolemia; Factor VII deficiency; Glycogen storage disease type I; infantile Refsum's disease; Progressive familial intrahepatic cholestasis type 2; hereditary tyrosinemia type 1; and various urea cycle defects; acute liver failure, including juvenile and adult patients with acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including cirrhosis and/or fibrosis; acute-on-chronic liver disease caused by one of the following acute events: alcohol consumption, drug ingestion, and/or hepatitis B flares. Thus, the patients may have one or more of these or other liver conditions.

In some instances, diseases and disorders treated according to the methods described herein may include hepatocyte-specific (hepatocyte-intrinsic) dysfunction. For example, the dysfunction, and the etiology of the disease and/or disorder, may be due to, or primarily attributable to, dysfunction of the endogenous hepatocytes present within the subject. In some instances, the hepatocyte-specific dysfunction may be genetic or inherited by the subject. In some instances, the etiology of the disease or disorder does not substantially involve cell types other than hepatocytes. In some instances, the disease or disorder results in decreased liver function, liver disease (acute or chronic), or other adverse condition derived from the endogenous hepatocytes. Accordingly, in some instances, e.g., where a disease is intrinsic to the endogenous hepatocyte population, an effective treatment may include replacement, supplementation, transplantation, or repopulation with hepatocytes as described herein. Without being bound by theory, in hepatocyte-intrinsic diseases/disorders replacement and/or supplementation of the endogenous hepatocytes can result in significant clinical improvement without the disease/disorder negatively impacting the transplanted hepatocytes. For example, where a subject has a genetic disorder affecting hepatocyte function (e.g., amino acid metabolism within hepatocytes, such as e.g., a hypertyrosinemia) allogenic transplanted hepatocytes may be essentially unaffected by the presence of the disease/disorder within the subject. Thus, transplanted hepatocytes may substantially engraft, survive, expand, and/or repopulate within the subject, resulting in a significant positive clinical outcome.

Diseases and disorders characterized by hepatocyte-specific (hepatocyte-intrinsic) dysfunction may be contrasted with diseases and disorders having an etiology that is not hepatocyte specific and involve hepatocyte extrinsic factors. Examples of diseases having factors and/or an etiology that is hepatocyte extrinsic include but are not limited to e.g., alcoholic steatohepatitis, alcoholic liver disease (ALD), hepatic steatosis/nonalcoholic fatty liver disease (NAFLD), and the like. Hepatocyte extrinsic diseases involve hepatic insults that are external, or derived from outside the endogenous hepatocytes, such as alcohol, diet, infection, etc. In some instances, diseases and disorders treated according to the methods described herein may include diseases and disorders that are not hepatocyte-specific (hepatocyte-intrinsic) dysfunction.

Examples of hepatocyte-intrinsic and hepatocyte-related diseases include liver-related enzyme deficiencies, hepatocyte-related transport diseases, and the like. Such liver-related deficiencies may be acquired or inherited diseases and may include metabolic diseases (such as e.g. liver-based metabolic disorders). Inherited liver-based metabolic disorders may be referred to “inherited metabolic diseases of the liver”, such as but not limited to e.g., those diseases described in Ishak, Clin Liver Dis (2002) 6:455-479. Liver-related deficiencies may, in some instances, result in acute and/or chronic liver disease, including e.g., where acute and/or chronic liver disease is a result of the deficiency when left untreated or insufficiently treated. Non-limiting examples of inherited liver-related enzyme deficiencies, hepatocyte-related transport diseases, and the like include Crigler-Najjar syndrome type 1; familial hypercholesterolemia, Factor VII deficiency, Glycogen storage disease type I, infantile Refsum's disease, Progressive familial intrahepatic cholestasis type 2, hereditary tyrosinemias (e.g., hereditary tyrosinemia type 1), genetic urea cycle defects, phenylketonuria (PKU), hereditary hemochromatosis, Alpha-I antitrypsin deficiency (AATD), Wilson Disease, and the like. Non-limiting examples of inherited metabolic diseases of the liver, including metabolic diseases having at least some liver phenotype, pathology, and/or liver-related symptom(s), include 5-beta-reductase deficiency, AACT deficiency, Aarskog syndrome, abetalipoproteinemia, adrenal leukodystrophy, Alpers disease, Alpers syndrome, alpha-1-antitrypsin deficiency, antithrombin III deficiency, arginase deficiency, argininosuccinic aciduria, arteriohepatic dysplasia, autoimmune lymphoproliferative syndrome, benign recurrent cholestasis, beta-thalassemia, Bloom syndrome, Budd-Chiari syndrome, carbohydrate-deficient glycoprotein syndrome, ceramidase deficiency, ceroid lipofuscinosis, cholesterol ester storage disease, cholesteryl ester storage disease, chronic granulomatous, chronic hepatitis C, Crigler-Najjar syndrome, cystic fibrosis, cystinosis, diabetes mellitus, Dubin-Johnson syndrome, endemic Tyrolean cirrhosis, erythropoietic protoporphyria, Fabry disease, familial hypercholesterolemia, familial steatohepatitis, fibrinogen storage disease, galactosemia, gangliosidosis, Gaucher disease, genetic hemochromatosis, glycogenosis type 1a, glycogenosis type 2, glycogenosis type 3, glycogenosis type 4, granulomatous disease, hepatic familial amyloidosis, hereditary fructose intolerance, hereditary spherocytosis, Hermansky-Pudlak syndrome, homocystinuria, hyperoxaluria, hypobetalipoproteinemia, hypofibrinogenemia, intrahepatic cholestasis of pregnancy, Lafora disease, lipoamide dehydrogenase deficiency, lipoprotein disorders, Mauriac syndrome, metachromatic leukodystrophy, mitochondrial cytopathies, Navajo neurohepatopathy, Niemann-Pick disease, nonsyndromic paucity of bile ducts, North American Indian childhood cirrhosis, ornithine transcarbamylase deficiency, partial lipodystrophy, Pearson syndrome, porphyria cutanea tarda, progressive familial intrahepatic cholestasis, progressive familial intrahepatic cholestasis type 1, progressive familial intrahepatic cholestasis type 2, protein C deficiency, Shwachman syndrome, Tangier disease, thrombocytopenia purpura, total lipodystrophy, type 1 glycogenosis, Tyrolean cirrhosis, tyrosinemia, urea cycle disorders, venocclusive disease, Wilson disease, Wolman disease, X-linked hyper-IgM syndrome, and Zellweger syndrome,

Treatment of subjects according to the methods described herein may result in various clinical benefits and/or measurable outcomes, including but not limited to e.g., prolonged survival, delayed disease progression (e.g., delayed liver failure), prevention of liver failure, improved and/or normalized liver function, improved and/or normalized amino acid levels, improved and/or normalized ammonia levels, improved and/or normalized albumin levels, improved and/or normalized bilirubin, recovery from a failure to thrive phenotype, reduction in lethargy, reduction in obtundation, reduction in seizures, reduction in jaundice, improved and/or normalized serum glucose, improved and/or normalized INR, improved and/or normalized urine test results, and the like.

For example, in some instances, administration of genetically modified hepatocytes and/or hepatocyte progenitors as described herein results in at least a 5% increase in survival of subjects having a liver disease and/or a condition resulting in liver failure as compared to e.g., subjects treated according to the standard of care and/or administered hepatocytes and/or hepatocyte progenitors that have not been genetically modified as described herein. The observed level of enhanced survival in such subject may vary and may range from an at least 5% to 60% or more increase, including but not limited to e.g., an at least 5%, at least 10%, at least 15%, at least 20%, at least 25%, at least 30%, at least 35%, at least 40%, at least 45%, at least 50%, at least 55%, at least 60% or more increase in survival. In some instances, subjects administered genetically modified hepatocytes and/or progenitors thereof as described herein may experience a delay in disease progression and/or the onset of one or more disease symptoms, such as but not limited to e.g., liver failure and/or any symptom(s) attributable thereto. Such a delay in disease progression and/or symptom onset may last days, weeks, months or years, including but not limited to e.g., at least one week, at least one month, at least 2 months, at least 3 months, at least 4 months, at least 5 months, at least 6 months, at least a year or more. The hepatocytes as described herein administered to a patient effect a beneficial therapeutic response in the patient over time.

Non-limiting examples of liver conditions that may be treated include acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis B, chronic hepatitis C, chronic hepatitis D, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, nonalcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis A, viral hepatitis B, viral hepatitis C, viral hepatitis D, viral hepatitis E, and zellweger syndrome, and the like. In some instances, a subject may be treated for fibrosis or a fibrotic condition. In some instances, a subject may be treated for cirrhosis or a cirrhotic condition.

Non-limiting examples of genetic conditions include: 1p36 deletion syndrome, 1q21.1 deletion syndrome, 2q37 deletion syndrome, 5q deletion syndrome, 5,10-methenyltetrahydrofolate synthetase deficiency, 17q12 microdeletion syndrome, 17q12 microduplication syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, Adenylosuccinate lyase deficiency, Adrenoleukodystrophy, Alagille syndrome, ADULT syndrome, Aicardi-Goutières syndrome, Albinism, Alexander disease, Alfi's syndrome, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dubé syndrome, Brody myopathy, Brunner syndrome, CADASIL syndrome, Cat eye syndrome, CRASIL syndrome, Chronic granulomatous disorder, Campomelic dysplasia, Canavan disease, Carpenter Syndrome, CDKL5 deficiency disorder, Cerebral dysgenesis-neuropathy-ichthyosis-keratoderma syndrome (CEDNIK), Cystic fibrosis, Charcot-Marie-Tooth disease, CHARGE syndrome, Chédiak-Higashi syndrome, Chondrodysplasia, Grebe type, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Currarino syndrome, Darier's disease, Dent's disease (Genetic hypercalciuria), Denys-Drash syndrome, De Grouchy syndrome, Down Syndrome, DiGeorge syndrome, Distal hereditary motor neuropathies, multiple types, Distal muscular dystrophy, Duchenne muscular dystrophy, Dravet syndrome, Edwards Syndrome, Ehlers-Danlos syndrome, Emanuel syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis type 1, Hemochromatosis type 2A, Hemochromatosis type 2B, Haemochromatosis type 3, Hemochromatosis type 4, Hemochromatosis type 5, Hemophilia A, Hemophilia B, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Jacobsen syndrome, Joubert syndrome, Juvenile primary lateral sclerosis (JPLS), Keloid disorder, KIFIA-Associated Neurological Disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Miller-Dieker syndrome, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phelan-McDermid syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Proximal 18q deletion syndrome, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von Hippel-Lindau disease, von Willebrand disease, Waardenburg syndrome, Warkany syndrome 2, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49,XXXXX), XXXXY syndrome (49,XXXXY), XYY syndrome (47,XYY), XXYY syndrome (48,XXYY), XYYY syndrome (48,XYYY), XXXY syndrome (48,XXXY), XYYYY syndrome (49,XYYYY), and Zellweger syndrome.

Genetic conditions include many lysosomal storage diseases. Non-limiting examples of lysosomal storage diseases include gangliosidosis (including e.g., GM2 gangliosidosis (Type A, Type O, Type AB) and GM1 gangliosidosis types 1, 2, and 3); Niemann-Pick diseases A, B, and C; Gaucher disease types 1, 2, and 3; Fabry disease; Metachromatic leukodystrophy; Globoid leukodystrophy; Multiple sulfatase deficiency; Alfa mannosidosis; Schindler disease; Aspartylglucosaminuria; Fucosidosis; Hurler syndrome; Scheie syndrome; Hurler-Scheie syndrome; Hunter syndrome; SanFilippo syndrome A, B, C, and D; Morquio syndrome A and B; Maroteaux-Lamy syndrome; Sly syndrome; Neuronal ceroid lipofuscinosis; Galactosialidosis; Infantile sialic acid storage disease; Salla disease; Sialuria; Sialidosis I and II; I-cell disease; Pseudo-Hurler-Polydystrophy; Mucolipidosis IV; Lysosomal Acid lipase deficiency; Pompe disease; Danon disease; Cystinosis, and the like. Causative mutations in genetic lysosomal storage diseases, and the genes and deficient enzymes associated with individual lysosomal storage diseases, are known and have been described, e.g., in Rajkumar & Dumpa. (2021) In: StatPearls. Treasure Island (FL): StatPearls Publishing (Available at www (dot) ncbi (dot) nlm (dot) nih (dot) gov/books/NBK563270/).

Genetic conditions include many urea cycle disorders (UCDs). Non-limiting examples of UCDs include N-acetylglutamate synthase deficiency (NAGS deficiency), Carbamoylphosphate synthetase I deficiency (CPS1 deficiency), Ornithine transcarbamylase deficiency (OTC deficiency), Citrullinemia type I (ASS1 deficiency), Argininosuccinic aciduria (ASL deficiency), Arginase deficiency (hyperargininemia, ARG1 deficiency), Ornithine translocase deficiency (ORNT1 deficiency, hyperornithinemia-hyperammonemia-homocitrullinuria syndrome), and Citrin deficiency.

Treatments described herein may be performed chronically (i.e., continuously) or non-chronically (i.e., non-continuously) and may include administration of one or more agents chronically (i.e., continuously) or non-chronically (i.e., non-continuously). Chronic administration of one or more agents according to the methods described herein may be employed in various instances, including e.g., where a subject has a chronic condition, including e.g., a chronic liver condition (e.g., chronic liver disease, cirrhosis, alcoholic liver disease, nonalcoholic fatty liver disease (NAFLD/NASH), chronic viral hepatitis, etc.), a chronic genetic liver condition (alpha-1 antitrypsin deficiency, Hereditary hemochromatosis, Wilson disease, etc.), chronic liver-related autoimmune conditions (e.g., primary biliary cirrhosis (PBC), primary sclerosing cholangitis (PSC), autoimmune hepatitis (AIH), etc.) etc. Administration of one or more agents for a chronic condition may include but is not limited to administration of the agent for multiple months, a year or more, multiple years, etc. Such chronic administration may be performed at any convenient and appropriate dosing schedule including but not limited to e.g., daily, twice daily, weekly, twice weekly, monthly, twice monthly, etc. In some instances, e.g., in the case of correction of a genetic condition or other persistent gene therapies, a chronic condition may be treated by a single or few (e.g., 2, 3, 4, or 5) treatments. Non-chronic administration of one or more agents may include but is not limited to e.g., administration for a month or less, including e.g., a period of weeks, a week, a period of days, a limited number of doses (e.g., less than 10 doses, e.g., 9 doses or less, 8 doses or less, 7 doses or less, etc., including a single dose).

An effective amount of a composition of therapeutic cells will depend, at least, on the particular method of use, the subject being treated, the severity of the affliction, the manner of administration of the composition, and the mechanism of action of the therapeutic cells. A “therapeutically effective amount” of a composition is a quantity of a specified reagent, e.g., therapeutic cells, sufficient to achieve a desired effect in a subject being treated.

In some instances, the amount of genetically modified hepatocytes administered to a subject may include e.g., at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least 40 billion, at least 50 billion, at least 60 billion, at least 70 billion, at least 80 billion, at least 90 billion, or at least 100 billion hepatocytes. Genetically modified hepatocytes may be delivered to a subject in need thereof in a single dose or in multiple doses.

The specific dose level and frequency of dosage for any particular subject may be varied and will depend upon a variety of factors, including the activity of the cells of the composition(s), the stability and length of action of the cells of the composition, the age, body weight, general health, sex and diet of the subject, mode and time of administration, drug combination(s) co-administered, and severity of the condition of the host undergoing therapy.

The above listed examples of therapies should not be construed as limiting and essentially any appropriate therapy resulting in the desired therapeutic outcome in subjects identified as described may be employed.

### Kits & Systems

Aspects of the present disclosure also include kits and systems and, in some instances, devices, for use therewith or therein. The kits and/or systems may include, e.g., one or more of any of the components described above with respect to the compositions and methods of the present disclosure. Kits and/or systems may be configured for use in the methods described herein. Encoded elements may be separately provided, e.g., as separate polypeptide-encoding polynucleotides, or may be combined, where appropriate, e.g., as a single polynucleotide encoding two or more separate polypeptides or non-coding nucleic acids. Accordingly, multiple encoded components may be provided on a single or multiple vectors, including e.g., where such multiple encoded components are under the control of shared (e.g., a single or a single set of) or separate (e.g. or individual or separate sets of) regulatory elements. Agents may be in separate vessels or may be combined, according to any described or appropriate combination, into shared vessels. Useful vessels include vials, tubes, syringes, bottles, bags, ampules, and the like. In some embodiments, useful kits may further include a device.

In some instances, the kits and/or systems of the present disclosure may comprise one or more modifying reagents, such as one or more reagents for genetic modification of a cell such as, e.g., one or more gene editing compositions, one or more transgene reagents, and/or the like.

In some instances, a kit and/or system may include a vector, such as e.g., a vector that includes a transgene encoding a gene product. Useful vectors may be integrating or non-integrating. In some instances, an employed vector may be an integrating vector where the integrating vector is sufficient for functional integration of the transgene into a hepatocyte or progenitor thereof. In some instances, a kit and/or system may include an editing composition, such as e.g., where the editing composition is sufficient to generate an HLA class I deficiency in a hepatocyte or progenitor thereof. In some instances, a kit and/or system may be configured for modifying hepatocytes or progenitors, e.g., through specific configuration of the components of the kits and/or systems, such as vessels, design elements, instructions, directions to internet-accessible media, the like, and/or combinations thereof. Such specific configurations may guide a user to employ components of the kit, such as one or more modifying reagents provided in the kit to generate genetically modified hepatocytes and/or progenitors thereof and to expand the produced genetically modified cells, e.g., in a bioreactor. In some instances, a kit may include components and/or instructions for preservation and/or preparation of genetically modified cells for shipping, e.g., to a facility where the cells may be expanded, e.g., in an in vivo bioreactor. In some instances, a kit may include an editing composition that includes a non-viral vector, such as e.g., an LNP, including e.g., where the vector is sufficient to generate an HLA class I deficiency. In some instances, the kit may include one or more reagents for cryopreservation of the genetically modified hepatocytes and/or progenitors thereof, including where such cryopreservation is performed before and/or after expansion of the genetically modified hepatocytes.

In addition to the above components, the kits and/or systems may further include (in certain embodiments) instructions for practicing the methods. These instructions may be present in the kits and/or provided with the systems in a variety of forms, one or more of which may be present in the kit and/or provided with a system. One form in which these instructions may be present is as printed information on a suitable medium or substrate, e.g., a piece or pieces of paper on which the information is printed, in the packaging of the kit and/or system, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

Notwithstanding the appended claims, the present disclosure is also defined by the following embodiments.

1. A method of generating hypoimmunogenic hepatocytes or progenitors thereof, the method comprising:
contacting a cell population comprising human hepatocytes or progenitors thereof with an editing composition under conditions sufficient to generate a human leukocyte antigen (HLA) class I deficiency in the hepatocytes or progenitors thereof; and
contacting the cell population with a transgene encoding at least one NK cell decoy receptor under conditions sufficient for expression of the transgene by the hepatocytes or progenitors thereof, thereby generating a population of hypoimmunogenic hepatocytes or progenitors thereof.

2. The method of embodiment 1, wherein the editing composition is a beta-2-microglobulin (B2M)-editing composition.

3. The method of embodiment 1 or 2, wherein the human hepatocytes or progenitors thereof are primary human hepatocytes.

4. The method of any of the preceding embodiments, further comprising introducing the generated population of hypoimmunogenic hepatocytes or progenitors thereof into a bioreactor.

5. The method of embodiment 4, wherein the bioreactor is an in vivo bioreactor and the in vivo bioreactor is maintained under conditions sufficient to produce an expanded population of hypoimmunogenic hepatocytes, optionally wherein the in vivo bioreactor is a mouse, rat, or pig.

6. The method of any of the preceding embodiments, further comprising expanding human hepatocytes or progenitors thereof in a bioreactor and extracting cells from the bioreactor after expansion to obtain the cell population comprising human hepatocytes or progenitors thereof, optionally wherein the bioreactor is a mouse, rat, or pig.

7. The method of embodiment 6, wherein the human hepatocytes or progenitors thereof expanded in the bioreactor are primary human hepatocytes.

8. The method of any of the preceding embodiments, wherein the cell population is contacted with the editing composition and the transgene simultaneously.

9. The method of any of embodiments 1 to 7, wherein the cell population is contacted with the editing composition before being contacted with the transgene, optionally wherein the hepatocytes or progenitors thereof of the cell population are expanded between being contacted with the editing composition and the transgene.

10. The method of any of embodiments 1 to 7, wherein the cell population is contacted with the transgene before being contacted with the editing composition, optionally wherein the hepatocytes or progenitors thereof of the cell population are expanded between being contacted with transgene and the editing composition.

11. The method of any of the preceding embodiments, wherein the at least one NK cell decoy receptor comprises CD47, a B2M-HLA-E fusion, or a combination thereof.

12. The method of any of the preceding embodiments, wherein contacting the cell population with the transgene comprises contacting the cell population with an integrating vector comprising the transgene, optionally wherein the integrating vector is a lentiviral vector.

13. The method of any of the preceding embodiments, wherein the editing composition comprises a CRISPR-Cas protein or a polynucleotide encoding the CRISPR-Cas protein and a guide RNA (gRNA) or a polynucleotide encoding the gRNA.

14. The method of any of the preceding embodiments, wherein contacting with the editing composition comprises contacting the cell population with a vector comprising reagents sufficient for disrupting a B2M locus of the hepatocytes or progenitors, optionally wherein the vector is a non-viral vector, optionally wherein the non-viral vector is a lipid nanoparticle (LNP).

15. The method of embodiment 14, wherein the vector:
encodes a Cas9 protein and a guide RNA (gRNA) targeting the B2M locus; or
comprises a ribonucleoprotein (RNP) comprising the Cas9 protein and the gRNA.

16. The method of any of the preceding embodiments, wherein the method further comprises contacting the cell population with an HLA class II-targeting composition under conditions sufficient to generate an HLA class II deficiency in the hepatocytes or progenitors.

17. The method of embodiment 16, wherein the HLA class II-targeting composition comprises an editing composition that, under sufficient conditions, edits a locus encoding a transcription factor or coactivator that causes expression of an HLA class II gene.

18. The method of embodiment 17, wherein the HLA class II-targeting composition comprises a class II, major histocompatibility complex, transactivator (CIITA)-editing composition that edits a CIITA locus.

19. The method of embodiment 18, wherein the CIITA-editing composition comprises a CRISPR-Cas protein or a polynucleotide encoding the CRISPR-Cas protein and a gRNA targeting the CIITA locus or a polynucleotide encoding the gRNA.

20. The method of any of embodiments 17 to 19, wherein contacting with the editing composition comprises contacting the cell population with a vector comprising reagents sufficient for disrupting the locus encoding the transcription factor or coactivator, optionally wherein the vector is a non-viral vector, optionally wherein the non-viral vector is a lipid nanoparticle (LNP).

21. The method of embodiment 20, wherein the vector:
encodes a Cas9 protein and a guide RNA (gRNA) targeting the locus; or
comprises an RNP comprising the Cas9 protein and the gRNA.

22. The method of any of the preceding embodiments, further comprising cryopreserving the generated hypoimmunogenic hepatocytes or progenitors thereof.

23. A method of treating a subject for a condition, the method comprising:
administering to the subject an effective amount of hypoimmunogenic hepatocytes or progenitors, wherein the hypoimmunogenic hepatocytes or progenitors each comprise an HLA class I deficiency and a transgene encoding at least one NK cell decoy receptor, optionally wherein the condition is a liver condition.

24. The method of embodiment 23, wherein the subject has a contraindication to immunosuppression.

25. The method of embodiment 23 or 24, wherein the hypoimmunogenic hepatocytes or progenitors thereof are generated according to the method of any of embodiments 1 to 22.

26. A non-human mammal comprising an engrafted cell population, the cell population comprising a plurality of hypoimmunogenic human hepatocytes or progenitors thereof, wherein each hepatocyte or progenitor of the plurality comprises an HLA class I deficiency and a transgene encoding at least one NK cell decoy receptor.

27. The non-human mammal of embodiment 26, wherein the engrafted cell population is an allogenic or heterologous cell population with respect to the non-human mammal.

28. The non-human mammal of embodiment 26 or 27, wherein the non-human mammal is an in vivo bioreactor, optionally wherein the in vivo bioreactor is a mouse, rat, or pig.

29. The non-human mammal of any of embodiments 26 to 28, wherein the hypoimmunogenic human hepatocytes or progenitors thereof are primary human hepatocytes.

30. The non-human mammal of any of embodiments 26 to 29, wherein the HLA class I deficiency comprises a B2M deficiency.

31. The non-human mammal of any of embodiments 26 to 30, wherein the at least one NK cell decoy receptor comprises CD47, a B2M-HLA-E fusion, or a combination thereof.

32. The non-human mammal of any of embodiments 26 to 31, wherein each hepatocyte or progenitor of the plurality further comprises an HLA class II deficiency, optionally wherein the HLA class II deficiency comprises a deficiency in a transcription factor or coactivator that causes expression of an HLA class II gene, optionally wherein the transcription factor or coactivator is CIITA.

33. A population of hepatocytes or progenitors thereof comprising an expanded population of hypoimmunogenic human hepatocytes or progenitors thereof isolated from the non-human mammal of any of embodiments 26 to 32.

34. The population of hepatocytes or progenitors thereof of embodiment 33, wherein the population of hepatocytes or progenitors thereof is cryopreserved.

35. A cell population comprising a plurality of hypoimmunogenic primary human hepatocytes, wherein each hepatocyte of the plurality comprises an HLA class I deficiency and a transgene encoding at least one NK cell decoy receptor.

36. The cell population of embodiment 35, wherein the HLA class I deficiency comprises a B2M deficiency.

37. The cell population of embodiment 35 or 36, wherein the at least one NK cell decoy receptor comprises CD47, a B2M-HLA-E fusion, or a combination thereof.

38. The cell population of any of embodiments 35 to 37, wherein each hepatocyte of the plurality further comprises an HLA class II deficiency, optionally wherein the HLA class II deficiency comprises a deficiency in a transcription factor or coactivator that causes expression of an HLA class II gene, optionally wherein the transcription factor or coactivator is CIITA.

39. A method of generating genetically modified human hepatocytes, the method comprising:
contacting a cell population comprising human hepatocytes or progenitors thereof with an integrating vector comprising a transgene encoding a gene product under conditions sufficient for functional integration of the transgene to produce genetically modified hepatocytes or progenitors thereof comprising the integrated transgene; and
transplanting the genetically modified hepatocytes or progenitors thereof into an in vivo bioreactor and maintaining the in vivo bioreactor under conditions sufficient for expansion of the genetically modified hepatocytes or progenitors to generate an expanded population of genetically modified human hepatocytes that express the gene product, optionally wherein the in vivo bioreactor is a mouse, rat, or pig.

40. The method of embodiment 39, wherein the human hepatocytes or progenitors thereof are primary human hepatocytes.

41. The method of embodiment 39 or 40, further comprising cryopreserving the expanded population of genetically modified human hepatocytes.

42. The method of any one of embodiments 39 to 41, wherein the transgene encodes a gene product selected from the group consisting of: Copper-transporting ATPase 2 (ATP7B), Hereditary hemochromatosis protein (HFE), Hemojuvelin, Hepcidin (HAMP), Transferrin receptor protein 2 (TFR2), Solute carrier family 40 member 1 (SLC40A1), Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase (ARG), alpha-1 antitrypsin (AAT), fumarylacetoacetate hydrolase (FAH), Argininosuccinate lyase (ASL), Argininosuccinate synthase (ASS), Ornithine translocase (ORNT1), citrin, UDP-glucuronosyltransferase 1A1 (UGT1A1), Transthyretin (TTR), Serine—pyruvate aminotransferase (AGXT), Complement factor H (CFH), and combinations thereof.

43. A method of treating a subject for a condition, the method comprising:
administering to the subject an effective amount of genetically modified human hepatocytes generated according to the method of any of embodiments 39 to 42.

44. The method of embodiment 43, wherein the condition is a liver condition.

45. The method of embodiment 43 or embodiment 44, wherein the condition is a genetic disease, optionally a monogenic disease.

46. The method of embodiment 45, wherein the condition is a Factor VIII deficiency and the transgene encodes Factor VIII.

47. The method of embodiment 46, wherein the condition is Hemophilia A.

48. The method of embodiment 45, wherein the condition is a Factor IX deficiency and the transgene encodes Factor IX.

49. The method of embodiment 48, wherein the transgene encodes a Padua variant Factor IX.

50. The method of embodiment 48 or 49, wherein the condition is Hemophilia B.

51. The method according to any one of embodiments 46 to 50, further comprising modulating coagulation in the subject.

52. The method of embodiment 43, wherein the condition is a urea cycle disorder (UCD) and the transgene encodes one or more urea cycle polypeptides.

53a. The method of embodiment 52, wherein the transgene encodes one or more urea cycle polypeptides that are rate-limiting in the metabolism of nitrogen waste.

53b. The method of embodiment 43, wherein the condition is a lysosomal storage disease, optionally Fabry Disease, and the transgene encodes an enzyme associated with the lysosomal storage disease, optional an alpha-galactosidase A polypeptide.

54. A non-human mammal comprising an engrafted cell population, the cell population comprising a plurality of genetically modified human hepatocytes, wherein each hepatocyte of the plurality comprises a functionally integrated transgene encoding a gene product.

55. The non-human mammal of embodiment 54, wherein the engrafted cell population is an in vivo expanded cell population, and the non-human mammal further comprises hepatocyte progeny of the genetically modified human hepatocytes.

56. The non-human mammal of embodiment 54 or 55, wherein the genetically modified human hepatocytes further comprise an HLA class I deficiency and a transgene encoding at least one NK cell decoy receptor.

57. The non-human mammal of embodiment 56, wherein each hepatocyte of the plurality further comprises an HLA class II deficiency, optionally wherein the HLA class II deficiency comprises a deficiency in a transcription factor or coactivator that causes expression of an HLA class II gene, optionally wherein the transcription factor or coactivator is CIITA.

58. The non-human mammal of any of embodiments 54 to 57, wherein the transgene encodes a gene product selected from the group consisting of: Copper-transporting ATPase 2 (ATP7B), Hereditary hemochromatosis protein (HFE), Hemojuvelin, Hepcidin (HAMP), Transferrin receptor protein 2 (TFR2), Solute carrier family 40 member 1 (SLC40A1), Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase (ARG), alpha-1 antitrypsin (AAT), fumarylacetoacetate hydrolase (FAH), Argininosuccinate lyase (ASL), Argininosuccinate synthase (ASS), Ornithine translocase (ORNT1), citrin, UDP-glucuronosyltransferase 1A1 (UGT1A1), Transthyretin (TTR), Serine—pyruvate aminotransferase (AGXT), Complement factor H (CFH), and combinations thereof.

59. The non-human mammal of any of embodiments 54 to 58, wherein the non-human mammal is an in vivo bioreactor.

60. The non-human mammal of embodiment 59, wherein the in vivo bioreactor is a rodent.

61. The non-human mammal of embodiment 59, wherein the rodent in vivo bioreactor is a rat in vivo bioreactor.

62. The non-human mammal of embodiment 59, wherein the in vivo bioreactor is a pig.

63. The non-human mammal of any one of embodiments 60 to 62, wherein the rodent in vivo bioreactor is deficient for interleukin 2 receptor subunit gamma (IL2rg), recombination activating gene 1 (RAG1), recombination activating gene 2 (RAG2), or a combination thereof.

64. The non-human mammal of any one of embodiments 60 to 63, wherein the rodent in vivo bioreactor is deficient for fumarylacetoacetate hydrolase (FAH).

65. The non-human mammal of any of embodiments 54 to 62, wherein the genetically modified human hepatocytes thereof are modified primary human hepatocytes.

66. A population of hepatocytes or progenitors thereof comprising an expanded population of genetically modified human hepatocytes isolated from the non-human mammal of any of embodiments 54 to 65.

67. The population of hepatocytes or progenitors thereof of embodiment 66, wherein the population of hepatocytes is cryopreserved.

68. The population of hepatocytes or progenitors thereof of embodiment 66 or 67, wherein the population comprises from 100 million to 20 billion hepatocytes or progenitors thereof.

69. The population of hepatocytes or progenitors thereof of any one of embodiments 66 to 68, wherein the hepatocytes or progenitors thereof are present in a container, optionally wherein the container is a culture vessel, a tube, a flask, a vial, a cryovial, or a cryo-bag.

70. A cell population comprising a plurality of hypoimmunogenic primary human hepatocytes, wherein each hepatocyte of the plurality comprises an HLA class I deficiency and a transgene encoding at least one NK cell decoy receptor.

71. The cell population of embodiment 70, comprising from 100 million to 20 billion of the hypoimmunogenic primary human hepatocytes.

72. The cell population of embodiment 70 or embodiment 71, wherein the cell population is present in a container, optionally wherein the container is a culture vessel, a tube, a flask, a vial, a cryovial, or a cryo-bag.

73. A method of generating a plurality of hepatocyte cell therapy doses, the method comprising:
(1a) genetically modifying human hepatocytes and expanding the genetically modified human hepatocytes in one or more in vivo bioreactors to generate an expanded population of genetically modified human hepatocytes, or
(1b) genetically modifying expanded human hepatocytes obtained from one or more in vivo bioreactors to generate an expanded population of genetically modified human hepatocytes; and
(2) aliquoting the expanded population of genetically modified human hepatocytes of 1a or 1b into a plurality of hepatocyte cell therapy doses.

74. The method of embodiment 73, wherein the plurality comprises at least 10 doses of at least 1 billion hepatocytes each, optionally at least 10 doses of at least 10 billion hepatocytes each, optionally at least 100 doses of at least 1 billion cells each.

75. The method of embodiment 73 or 74, wherein the human hepatocytes are derived from a single human liver.

76. A method of treating a plurality of subjects having a condition, the method comprising: generating a plurality of hepatocyte cell therapy doses according to any of embodiments 73 to 75; and administering one or more doses of the plurality to each of the subjects to treat the subjects for the condition.

77. The method of embodiment 76, wherein the plurality of subjects comprises at least 10 subjects, optionally at least 100 subjects.

78. The method of embodiment 76 or 77, wherein each subject of the plurality are treated for the same condition.

79. The method of embodiment 76 or 77, wherein two or more subjects of the plurality are treated for different conditions.

80. A kit or system comprising:
one or more modifying reagents comprising:
a vector comprising a transgene encoding a gene product, the vector sufficient for functional integration of the transgene into a hepatocyte or progenitor thereof; and/or
an editing composition sufficient to generate an HLA class I deficiency in the hepatocyte or progenitor thereof; and
optionally, instructions for modifying hepatocytes or progenitors thereof using the one or more modifying reagents to generate genetically modified hepatocytes or progenitors thereof and expanding the genetically modified cells in a bioreactor.

81. The kit or system of embodiment 80, wherein the vector is an integrating vector sufficient for functional integration of the transgene into a hepatocyte or progenitor thereof.

82. The kit or system of embodiment 80 or 81, wherein the editing composition comprises a non-viral vector, optionally an LNP, sufficient to generate the HLA class I deficiency. 83. The kit or system of any of embodiments 80 to 82, further comprising one or more reagents for cryopreservation of the genetically modified hepatocytes or progenitors thereof.

### Examples

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

General methods in molecular and cellular biochemistry can be found in such standard textbooks as Molecular Cloning: A Laboratory Manual, 4th Ed. (Sambrook et al., Cold Spring Harbor Laboratory Press 2012); Short Protocols in Molecular Biology, 4th Ed. (Ausubel et al. eds., John Wiley & Sons 1999); Protein Methods (Bollag et al., John Wiley & Sons 1996); and Cell and Tissue Culture: Laboratory Procedures in Biotechnology (Doyle & Griffiths, John Wiley & Sons 1998), the disclosures of which are incorporated herein by reference. Reagents, antibodies, cells, tissue samples, etc., and kits referred to in this disclosure are available from commercial vendors such, but not limited to, those vendors identified herein.

### Example 1: Generation of Universal Human Hepatocytes

Universal human hepatocytes were generated by ex vivo engineering of primary human hepatocytes (PHH) (1) to be deficient in human leukocyte antigen (HLA) class I, thereby blocking recognition by cytotoxic T cells (CTLs) in vivo, and (2) to express a decoy receptor for NK cells, thereby inhibiting killing by natural killer (NK) cells in vivo. HLA class I deficiency was achieved by CRISPR/Cas9 targeted knock-out of beta-2-microglobulin (B2M) and NK cell decoy receptor expression was achieved by transduction with a lentiviral vector carrying a transgene encoding either CD47 or a B2M-HLA-E fusion construct.

Various methods to deliver gene editing reagents to PHH were employed and evaluated, including but not limited to the production of Cas9 and synthetic chemically modified gRNA (Synthego) containing ribonucleoprotein (RNP) delivered to cells by transfection or nucleofection. For example, RNP transfection was performed using the CRISPRMAX™ Cas9 system (ThermoFishser Scientific) according to manufacturer's instructions. Briefly, TrueCut™ Cas9 Protein v2 (ThermoFisher Scientific) and synthetic chemically modified sgRNA (Synthego) were complexed into RNP, then added, along with the CRISPRMAX™ reagents, directly to freshly thawed PHH, then rocked in a 37 deg. C incubator for 2 hrs. RNP nucleofection was performed on a Lonza 4D-Nucleofector™ X Unit using the P3 Primary Cell 4D-Nucleofector Kit according to manufacturer's instructions.

An exemplary gRNA sequence targeting exon 1 of the B2M locus used in this example is as follows:

| Name | Target Sequence | PAM sequence |
|---|---|---|
| B2M_Ex1_7 | GGCCACGGAGCGAGACATCT | CGG |
| | (SEQ ID NO: 039) | |

As a control, the irrelevant safe harbor locus, AAVS1, was targeted using the same CRISPR/Cas9 systems described above but employing AAVS1-targeting gRNA having the following sequence:

| Name | Target sequence | PAM sequence |
|---|---|---|
| AAVS1 | GGGGCCACTAGGGACAGGAT | TGG |
| | (SEQ ID NO: 042) | |

Following editing, cells subjected to nucleofection or transfection protocols were transduced with lentiviral vector (LVV) containing either a CD47 or B2M-HLA-E fusion transgene for two hours with rocking at 37 deg. C. Sequences of the transgenes employed are as follows:

| Name | Sequence |
|---|---|
| hsCD47 | SEQ ID NO: 043 (encoding SEQ ID NO: 037) |
| Truncated CD47 | SEQ ID NO: 045 (encoding SEQ ID NO: 038) |
| B2M-HLA-E | SEQ ID NO: 047 (encoding SEQ ID NO: 036) |
| fusion | |

Negative control mock-transductions with two hours of rocking without the addition of LVV were also performed, allowing for assessment of B2M or control locus editing in the absence of LVV transduction.

Editing efficiency at the targeted B2M locus, or the AAVS1 control locus, was assessed. Briefly, genomic DNA (gDNA) was extracted from cell samples and the region of interest was PCR amplified. Amplified DNA was sequenced by Sanger method and the resulting reads were subjected to Tracking of Indels by Decomposition (TIDE) analysis. Resulting protein expression levels, of B2M and/or expressed transgene, in cells subjected to either targeted or control editing was evaluated by flow cytometry using anti-HLA-ABC, anti-HLA-E, and/or anti-CD47 specific antibodies.

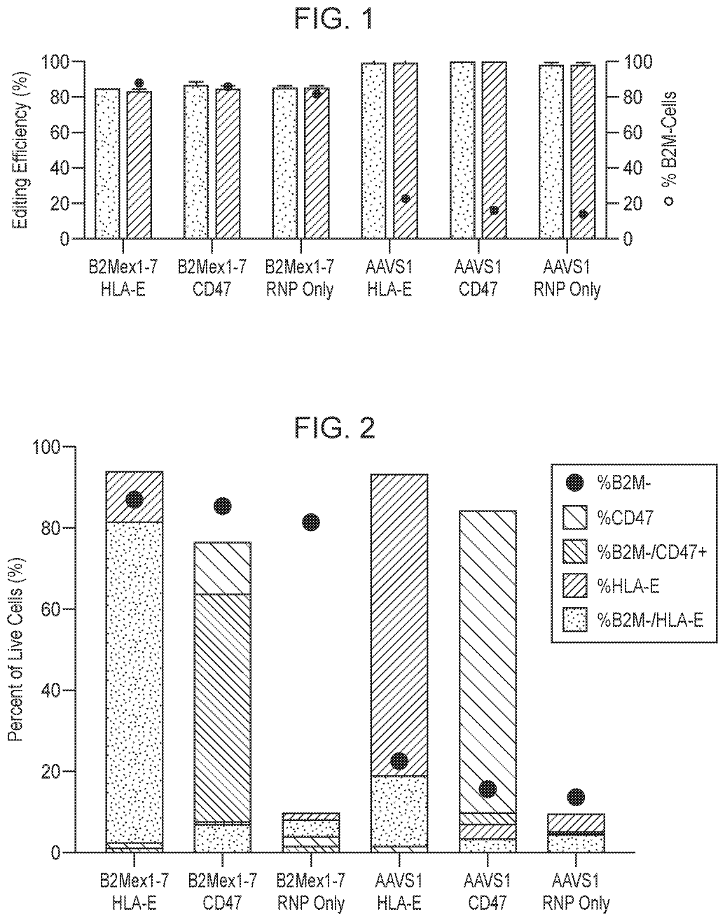

As shown in FIG. 1, editing efficiencies of 80% or greater, measured by indels (left y-axis, black bars) or knock-out (KO) score (left y-axis, gray bars), were observed at both the B2M target locus and the AAVS1 control locus, respectively. Editing efficiencies with B2M exon 1 targeting reagents alone (“B2Mex1-7 RNP Only”) or control locus targeting reagents alone (“AAVS1 RNP Only”) were similar to efficiencies observed when either NK cell decoy receptor LVV transduction, i.e., B2M-HLA-E fusion (“B2Mex1-7 HLA-E”; “AAVS1 HLA-E”) or CD47 (“B2Mex1-7 CD47”; “AAVS1 CD47”), was included. Furthermore, the percentage of B2M negative cells (“B2M-”) as measured by flow cytometry (right y-axis, red dots) indicated that at least 80% of cells in the B2M edited samples (“B2Mex 1-7 HLA-E”, B2Mex1-7 CD47″, and “B2Mex1-7 RNP Only”) did not express B2M protein. In comparison, less than 30%, and in some cases less than 20%, of cells in the AAVS1 control samples (“AAVS1 HLA-E”, “AAVS1 CD47”, and “AAVS1 RNP Only”) were B2M negative. These data demonstrate the successful production of B2M negative PHH through the editing process described, which achieves high editing efficiency and specificity of B2M KO at both the genomic and protein levels.

In addition to B2M KO, expression of the introduced NK decoy receptor transgenes, CD47 and HLA-E, was also assessed in the treated PHH. Results of flow cytometric analysis of both B2M negativity as well as HLA-E or CD47 expression in the aforedescribed groups (“B2Mex1-7 HLA-E”; “B2Mex1-7 CD47”; “B2Mex1-7 RNP Only”; “AAVS1 HLA-E”; “AAVS1 CD47”; “AAVS1 RNP Only”) are provided in FIG. 2. As shown, high levels of cells that are simultaneously negative for B2M and positive for decoy receptor expression (“% B2M−/HLA-E” or “% B2M−/CD47+”) were observed in the groups that were subjected to both B2M targeted editing and decoy receptor transduction. Correspondingly, low levels of B2M negativity were observed in control editing reactions (“AAVS1 HLA-E”; “AAVS1 CD47”; “AAVS1 RNP Only”) and decoy receptor was essentially absent in reactions not treated with LVV (“B2Mex1-7 RNP Only”; “AAVS1 RNP Only”). These data demonstrate the successful and efficient production of PHH that are both B2M negative and express one of two different NK cell decoy receptors.

In addition, these data demonstrate that the above-described approach results in populations of PHH that contain over 50%, and in some cases at least 60%, at least 70%, or at least 80% double-engineered PHH that are deficient in HLA class I while expressing an NK cell decoy receptor. Such percentages are not considered to be limiting and, accordingly, populations having greater than 80% double-engineered hepatocytes can be readily achieved through these and other methods described herein.

### Example 2: Double-Engineered Primary Human Hepatocytes are Hypoimmunogenic

Double-engineered PHH, having a B2M KO and expressing either CD47 or B2M-HLA-E fusion transgene, were produced essentially as described above. The double engineered cells were mixed with various immune cell compositions at various ratios and survival assays were performed. Briefly, the engineered cells were mixed with immune cells, including cytotoxic T lymphocytes (CTLs) and natural killer (NK) cells, activated by cytokine stimulation and having strong effector function and the survival (i.e., viability) of the double engineered cells was evaluated over time.

For example, survival in a 2:1 immune-to-target-cell co-culture was assessed for four different target cell groups: (1) double-engineered B2M exon 1 KO+B2M-HLA-E transgene cells, (2) double-engineered B2M exon 1 KO+CD47 transgene cells, (3) single engineered B2M exon 1 KO RNP only cells, and (4) AAVS1 RNP only control cells. The immune cells included a mixture of effector cells made primarily of CTLs and survival was assessed by a quantitative imaging-based cell viability assay over 72 hours. Substantial increases in survival were observed in all B2M knockout (B2M−; groups 1, 2, 3) PHH across all time points (24 hr, 48 hr, and 72 hr) as compared to the control group (4).

In another example, survival in a 2:1 immune-to-target-cell co-culture, where the immune cell mixture contained primarily NK cells, was assessed for four different groups of target cells: (1) double-engineered B2M exon 1 KO+B2M-HLA-E transgene cells, (2) double-engineered B2M exon 1 KO+CD47 transgene cells, (3) single engineered B2M exon 1 KO RNP only cells, and (4) AAVS1 RNP only control cells. Both double-engineered groups (1) and (2) showed increased levels of survival across the 24, 48, and 72 hr time points as compared to the control group (4); while the single-engineered group (3) showed a decrease in survival compared to the control group due to the increased “missing-self recognition” by NK cells caused by B2M knockout.

These data show that PHH, doubly engineered to be B2M negative and express an NK cell decoy receptor transgene, are subjected to reduced immune cell killing as compared to levels of immune cell killing seen in non-engineered cells and cells engineered only with B2M KO. Collectively, these assays demonstrate that engineering HLA class I deficiency and NK cell decoy receptor expression into PHH generates hypoimmunogenic PHH that show increased survival in the presence of activated immune effector cell populations, including populations that contain activated CTL and NK cell subpopulations.

### Example 3: Liver Repopulation with Hypoimmunogenic Engineered Primary Human Hepatocytes

Engineered PHH were generated by CRISPR/Cas9 KO of B2M through RNP transfection or nucleofection, with or without LVV transgene transduction, essentially as described above. Four different groups of engineered cells, (1) Cas9 B2M KO RNP via transfection, (2) Cas9 B2M KO RNP via transfection+LVV, (3) Cas9 B2M KO RNP via nucleofection, and (4) Cas9 B2M KO RNP via nucleofection+LVV, were separately transplanted into recipient FRGN mice via intrasplenic injection at 5×10⁵ viable cells per animal. Animals were subjected to cycling with NTBC to introduce selective pressure and promote engraftment of transplanted cells. Human albumin levels (hALB), as a surrogate for transplanted engineered PHH engraftment and expansion, were assessed at 2, 4, and 8 weeks post-transplantation.

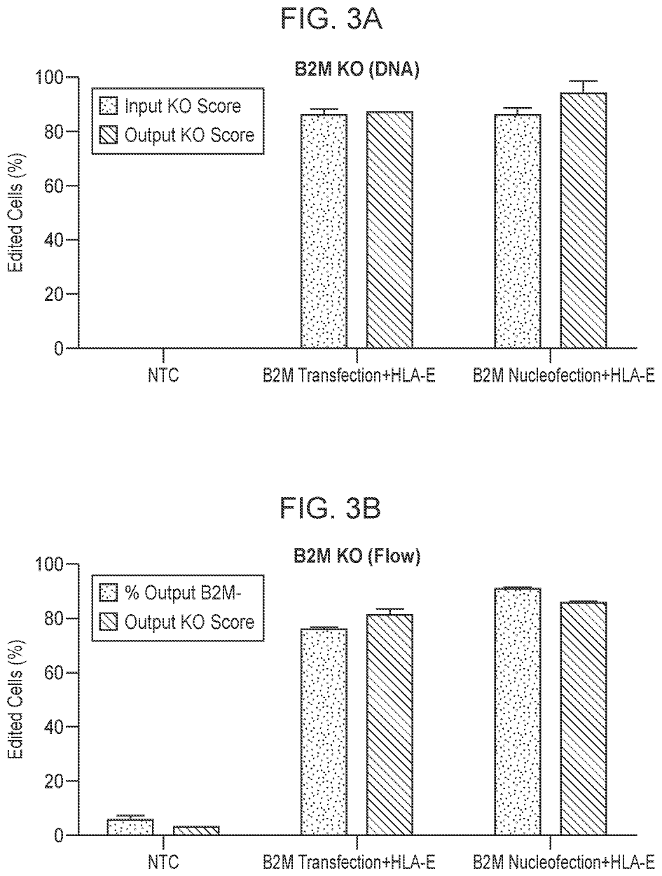

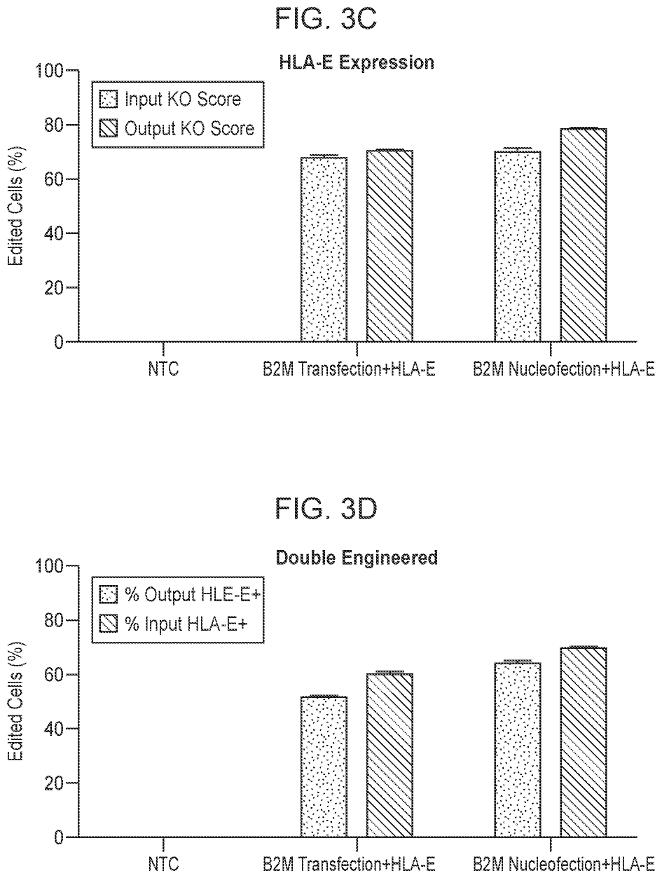

Levels of hALB were observed to increase in all groups over all three time points, indicating that engineered cells of all groups (1)-(4) were able to engraft and expand in the recipient animals. Furthermore, hALB levels were comparable, at corresponding timepoints, between transfection and nucleofection modes of RNP delivery, indicating that either delivery method of editing components can be successfully employed to generate functional engineered PHH capable of liver engraftment and repopulation. In addition, the representation of cells having the desired engineered characteristics (i.e., B2M KO, HLA-E transgene expression, or both KO and transgene expression) within the input population, i.e., ex vivo engineered PHH used for transplantation into the FRGN bioreactor, was compared to the output population, i.e., hepatocytes purified from the repopulated FRGN bioreactor following in vivo expansion. FIG. 3A-3D, provides the percent of desired engineered cells (generated using transfection or nucleofection) from input and output populations measured as having B2M KO by DNA analysis (FIG. 3A), B2M KO by flow cytometric analysis (FIG. 3B), HLA-E transgene expression by flow cytometric analysis (FIG. 3C), and double modification (i.e., both B2M KO and transgene expression) by flow cytometric analysis (FIG. 3D). Samples from, no-treatment-control (NTC) animals (i.e., animals transplanted with unmodified PHH) were also assessed in parallel. As shown, these comparisons revealed surprisingly similar representation of the desired engineered cells between input and output populations, indicating comparable engraftment, expansion, and repopulation kinetics between engineered and unmodified hepatocytes. In addition, at study termination (24 weeks post-transplant) host FRGN livers transplanted with engineered PHH showed similar levels of repopulation and humanization as compared to NTC animals that received unmodified PHH (as measured by liver immunohistochemistry for FAH and hAlb ELISA). For example, two representative animals transplanted with engineered cells showed 89.76% and 89.54% repopulation with FAH+ cells and 12,489 μg/mL and 11,615 μg/mL levels of hAlb as compared to 90.81% FAH+ cells and 11,607 μg/mL hAlb as observed in a representative NTC animal.

Collectively, these data show that hypoimmunogenic PHH, as well as PHH generally, engineered with a genomic edit and/or an integrated transgene according to the methods described herein are functional and capable of repopulating a recipient liver. Such repopulation was seen to occur at kinetics comparable to unedited/unmodified cells, indicating that hepatocytes engineered in this way, and their progeny, will persist in a host liver. Thus, universal hepatocytes, and hepatocytes generally engineered with any edit or transgene, as described herein may be, e.g., transplanted and successfully expanded in an in vivo bioreactor, transplanted into a subject for therapeutic purposes, and the like.

### Example 4: Transgene-Engineered PHH Engraft, Expand, and Produce Physiologically Relevant Amounts of Therapeutic Transgene Product In Vivo

In vivo FRG rat expanded human hepatocytes (huFRG) were isolated and cryopreserved. For cryopreservation, hepatocyte cell suspension was aliquoted into vessels and pelleted by centrifugation. Cell pellets were gently resuspended in cryopreservation media under cold conditions to reach a desired final concentration, such as e.g., 10 million live cells per mL, and the resuspended cells were kept at 4-8 deg. C. Hepatocytes prepared for cryopreservation were aliquoted into freezing containers and frozen using a controlled rate freezer. After controlled rate freezing was complete, cryopreserved hepatocytes were transferred to vapor phase liquid nitrogen for storage. Cryopreserved huFRG hepatocytes were thawed and transduced via lentiviral vector with an expression cassette encoding either human factor IX (i.e., F9 or FIX) or firefly luciferase (i.e., Luc) as a marker/control.

The lentiviral vector (LakePharma/Curia) F9 expression construct employed in this example included the MND promoter (SEQ ID NO:001) operably linked to an F9 coding sequence (SEQ ID NO:002), encoding an F9 Padua variant polypeptide (SEQ ID NO:003), operably linked to a 3′LTR (SEQ ID NO:004).

The lentiviral vector (Imanis LV050L) Luc expression construct employed in this example included an SFFV promoter (SEQ ID NO:005) operably linked to a Luc coding sequence (SEQ ID NO:006), encoding a Luc polypeptide (SEQ ID NO:007), and a EmGFP coding sequence (SEQ ID NO: 008), encoding a EmGFP polypeptide (SEQ ID NO:009), operably linked to a 3′LTR (SEQ ID NO:004).

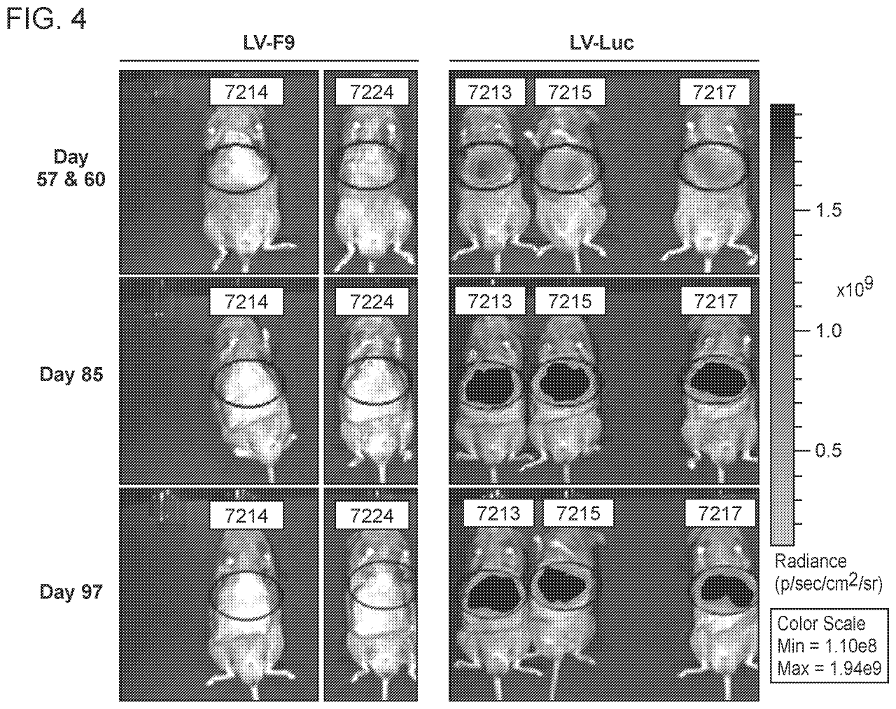

Following transduction, the transduced huFRG hepatocytes were transplanted into FRGN recipient mice via intrasplenic injection and the mice were maintained under conditions sufficient for engraftment and expansion of the transplanted huFRG hepatocytes. Mice transplanted with either F9-encoding lentiviral vector (hereafter, “LV-F9 mice”) or luciferase-encoding lentiviral vector (hereafter, “LV-Luc mice”) were subsequently assayed for luciferase bioluminescence at various timepoints during expansion of the transplanted cells within the host mouse livers using an IVIS live animal bioluminescence imaging system (PerkinElmer, Waltham, Massachusetts, USA). FIG. 4 provides representative IVIS images of LV-F9 and LV-Luc mice at day 57-60, day 85 and day 97 following transplantation, showing substantial bioluminescence in LV-Luc mice with increasing intensity at later time points. In this assay, the LV-F9 mice serve as a useful negative control because the LV-F9 vector does not encode for luciferase and thus no bioluminescence is expected to be detected in LV-F9 mice.

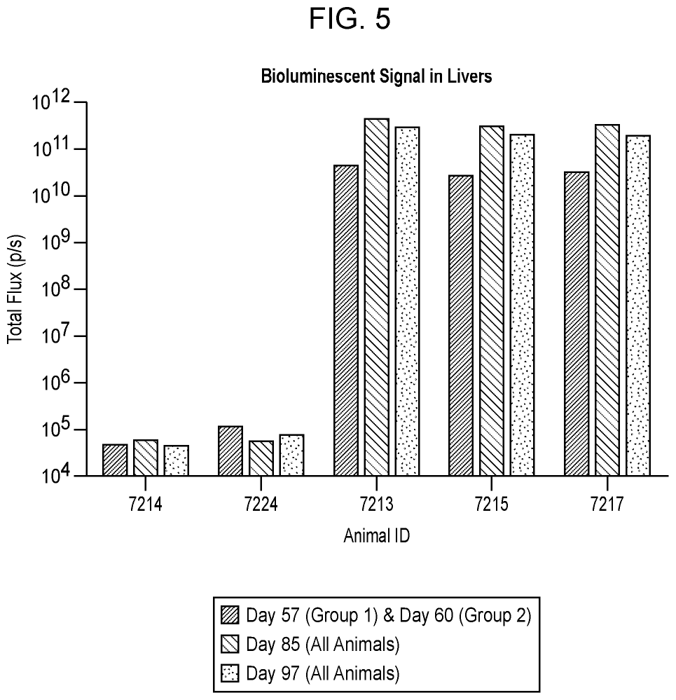

Bioluminescence measured on the IVIS was quantitated and FIG. 5 provides such quantification (measured as total flux in photons per second; p/s) of individual LV-F9 and LV-Luc mice at day 57 or 60, day 85 and day 97 following transplantation. The quantification confirms the qualitative observations described above, namely that the LV-Luc animals displayed substantial bioluminescence, e.g., as compared to LV-F9 animals, and the bioluminescence intensity was greater at the later timepoints as compared to the early, day 57 or 60, timepoints. Collectively, these findings demonstrate the effective engraftment of transduced huFRG hepatocytes in host mice and that the introduced transgene, luciferase in this case, was successfully and persistently expressed from the engrafted hepatocytes, and/or their progeny, for at least months following transplantation.

Employing similar methods as above, huFRG hepatocytes were transduced with a lentiviral vector containing an expression cassette encoding the Padua variant of human F9 (aka the “R338L” substitution (Simioni, et al. N Engl J Med 2009; 361:1671-5) corresponding to R384L substitution (as compared to wildtype UniProt P00740; RefSeq NP_000124.1; SEQ ID NO: 010), which displays eight times (8×) coagulation activity above normal physiological levels (see Lozier. Blood (2012) 120 (23): 4452-4453). 500,000 transduced huFRG hepatocytes were transplanted into FRGN recipient mice via intrasplenic injection and the mice were maintained under conditions sufficient for engraftment and expansion of the transplanted huFRG hepatocytes. Human albumin and human F9 levels were measured in blood samples collected from the LV-F9 mice at various timepoints following transplantation. Mice transplanted with huFRG hepatocytes transduced with LV-Luc were employed as controls and corresponding human albumin and human F9 measurements were collected from LV-Luc control animals.

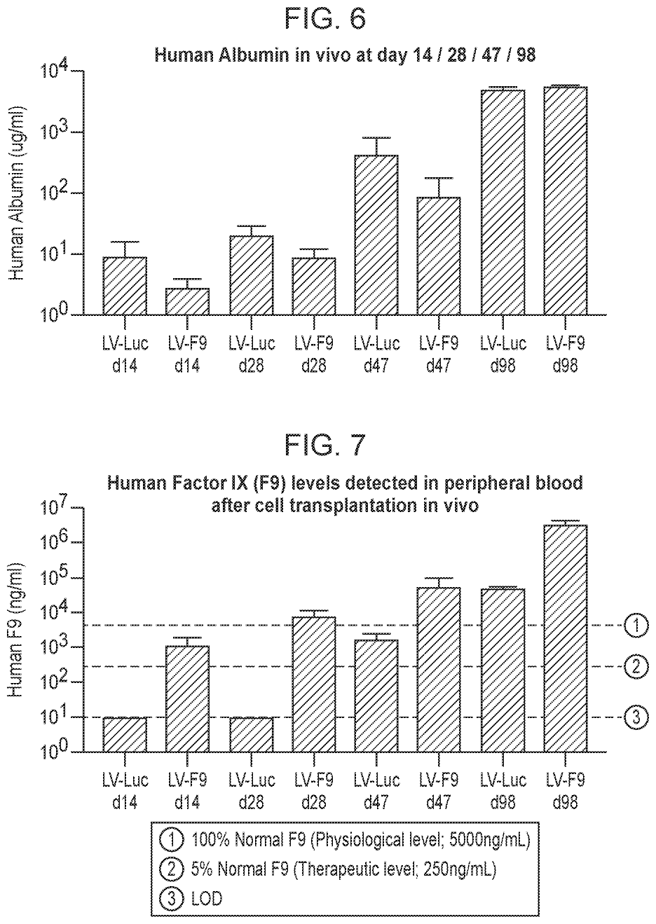

FIG. 6 provides the levels of heterologous human albumin (in micrograms per milliliter, log scale) as measured in peripheral blood samples from LV-F9 and LV-Luc mice collected at 14, 28, 47, and 98 days following transplantation. As can be readily seen, the levels of human albumin increased steadily in both cohorts, indicating similar levels of engraftment and expansion of LV-F9 and LV-Luc huFRG engineered hepatocytes in the respective host livers. In addition, the human albumin levels ultimately reached levels consistent with at least 70-80% humanization by 98 days, indicating robust in vivo engraftment and expansion of the ex vivo engineered hepatocytes.

FIG. 7 provides the levels of heterologous human F9 (in nanograms per milliliter, log scale) as measured in peripheral blood samples from LV-F9 and LV-Luc mice collected at 14, 28, 47, and 98 days following transplantation. The lower limit of detection (LOD) of the assay is indicated by a horizontal dotted line. As a proxy to evaluate the therapeutic potential of engineered huFRG hepatocytes in clinical deficiencies (e.g., as observed in monogenic diseases), F9 levels corresponding to (1) those necessary to achieve a desired therapeutic effect in F9-deficient human subjects (i.e., 5% of normal physiological level, 250 ng/ml, “5% normal F9”) or (2) a normal physiological level in human subjects (i.e., 100% of normal physiological level, 5000 ng/mL, “100% normal F9”) are also indicated by horizontal dotted lines.

As can be readily seen in FIG. 7, LV-F9 mice reached human F9 levels exceeding that necessary for a desired therapeutic effect at least as early as the first timepoint evaluated (i.e., 14 days) post-transplantation. Moreover, the LV-F9 mice reached human F9 levels exceeding 100% of normal physiological levels by at least day 28 post-transplantation. Such mice continued to display super-physiological levels of human F9 at all following timepoints.

Collectively, these findings demonstrate that huFRG hepatocytes, ex vivo engineered to express human F9, readily engraft, expand, and produce detectable levels of human F9 in recipient peripheral blood. Moreover, the transplanted mice rapidly reached levels of human F9 in peripheral blood that correspond to levels sufficient for therapeutic efficacy in human F9-deficiency. In addition, levels corresponding to, and even exceeding, 100% of normal human physiological F9 levels were achieved and persisted through the last measured timepoint.

HuFRG hepatocytes transplanted into LV-Luc mice contain an endogenous human gene encoding Factor IX. Thus, although these cells do not carry a heterologous F9 transgene like the LV-F9 huFRG hepatocytes, the Luc hepatocytes nonetheless express human F9 from the endogenous locus. While initial (i.e., day 14 and 28 post-transplantation) levels of human F9 in peripheral blood collected from LV-Luc mice were at or below the LOD (see e.g., FIG. 7), human F9 levels did eventually reach significant levels at later timepoints (see e.g., FIG. 7, LV-F9 d47 and d98) following substantial expansion of huFRG cells within the host liver (as confirmed by measuring human albumin levels). In comparison to such human F9 production in LV-Luc mice, human F9 production in LV-F9 mice was substantially higher at each timepoint, indicating more rapid achievement of therapeutic and physiological levels as well as overall greater levels at the final timepoint measured. For example, while LV-F9 mice reached 100% of normal physiological levels of F9 by day 28, LV-Luc mice did not reach 100% of normal physiological levels until the day 98 timepoint. Accordingly, these data indicate that the presence of the F9 transgene provides a significant advantage in both onset and potency of the therapeutic effect.

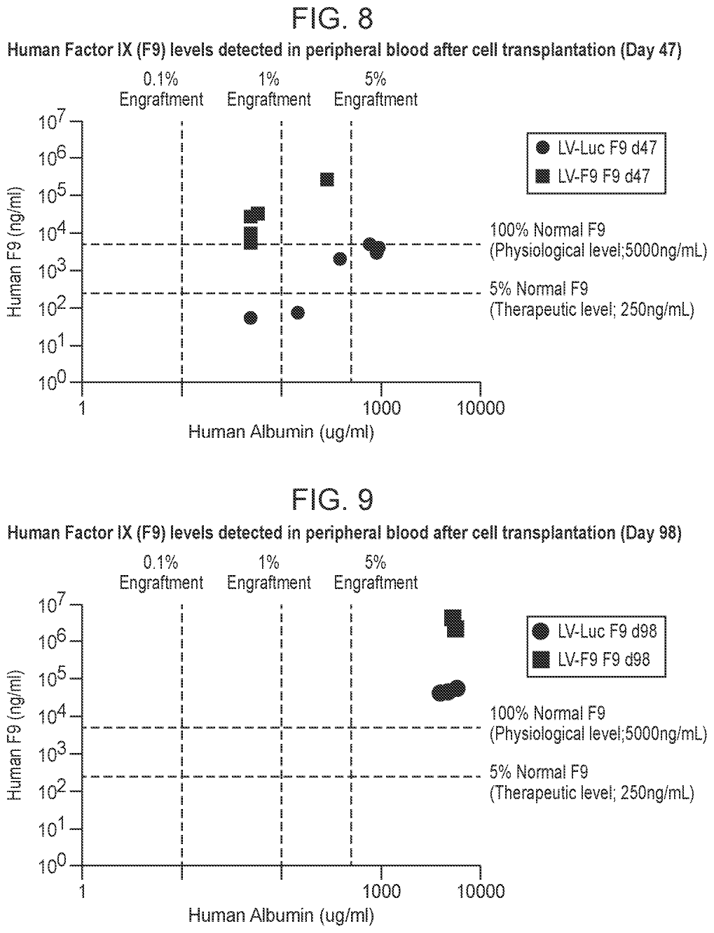

FIG. 8 provides a plot of human F9 levels measured in each animal versus the corresponding human albumin level in each animal at the day 47 time point. Reference levels for 0.1%, 1%, and 5% engraftment as well as for 5% and 100% of normal physiological human F9 are shown as vertical and horizontal dotted lines, respectively. In all cases, when mice having substantially similar levels of engraftment were compared, those that received huFRG hepatocytes engineered ex vivo with the Padua F9 transgene had higher human F9 levels in peripheral blood as compared to corresponding LV-Luc mice. Accordingly, the data further supports higher per cell levels of F9 expression in those cells that received the F9 transgene, e.g., as compared to cells expressing F9 from an endogenous locus. Moreover, this analysis demonstrates that less than 1% engraftment, and even as low as 0.2% engraftment, of huFRG hepatocytes ex vivo engineered to express a human F9 transgene is sufficient to achieve both therapeutic and even normal physiological concentrations of human F9 in peripheral blood.

FIG. 9 provides the corresponding plot to FIG. 8 for animals at the day 96 timepoint. Despite similar levels of engraftment in the LV-Luc and LV-F9 animals (indicated by substantially similar positions on the x-axis of all data points), peripheral blood of the LV-F9 animals contained about 60 times (60×) more human F9 as compared to peripheral blood from the LV-Luc animals. Considering this high level of expression and the enhanced coagulation activity of the Padua variant, the LV-F9 mice display a theoretical coagulation activity 490 times (490×) greater than that of the LV-Luc control animals.

Collectively, these data demonstrate the successful engraftment and expansion of human hepatocytes engineered to contain and express a therapeutic transgene within a host liver. Moreover, these data demonstrate that the engraftment and expansion of engineered hepatocytes carrying a transgene (regardless of the identity of the transgene) are at least comparable to the engraftment and expansion seen in control cells not carrying a transgene. Furthermore, given the observed high levels of expression of therapeutic factors from transgene engineered hepatocytes (e.g., as compared to corresponding expression of related endogenous factors in non-engineered cells), engraftment and expansion of such engineered hepatocytes, as described herein, provides for rapid achievement of therapeutically relevant levels of the transgene expression product that increases and persist over time, including over multiple months.

### Example 5: Generation and Expansion of Factor IX Engineered Human Hepatocytes for Hemophilia B

The following expression constructs were designed for introduction into human hepatocytes to facilitate expression of the therapeutic transgene product by engineered hepatocytes transplanted into subjects in need thereof, such as human subjects having a Factor IX deficiency such as Hemophilia B.

F9 expression constructs employed in this example include a suitable promoter, such as e.g., the MND promoter (SEQ ID NO:001), operably linked to an F9 coding sequence, such as e.g., a full-length F9 coding sequence (SEQ ID NO:011), encoding a full-length F9 polypeptide (SEQ ID NO:010), a Padua variant F9 coding sequence (SEQ ID NO:002), encoding an F9 Padua variant polypeptide (SEQ ID NO:003), or the like, operably linked to a suitable 3′ sequence, including e.g., a polyadenylation signal (polyA).

As will be readily understood, in some instances, substitutions may be made in the above-described constructs including e.g., exchange of the described promoter for another appropriate promoter, exchange of the transgene coding sequence for another coding sequence encoding the same transgene or a variant of the transgene, exchange of the sequence 3′ of the transgene for another 3′ sequence (e.g., including an alternative polyA or other 3′ components), or the like. Expression constructs are introduced into a suitable lentiviral vector for transduction into hepatocytes.

Freshly isolated human hepatocytes, or recently thawed cryopreserved hepatocytes, are transduced with one of the above-described expression constructs. Useful freshly isolated human hepatocytes include those isolated from cadaveric donor liver tissue as well as those expanded in, and isolated from, an in vivo bioreactor. Useful cryopreserved hepatocytes include those cryopreserved following isolation from cadaveric donor liver tissue as well as those cryopreserved following expansion in, and isolation from, an in vivo bioreactor. Accordingly, transduction is performed before or after expansion of the human hepatocytes in an in vivo bioreactor, such as e.g., a rodent bioreactor.

Human hepatocytes transduced with any of the above constructs may be otherwise unmodified, where, e.g., introduction of the above construct is the only genetic modification performed. Alternatively, human hepatocytes transduced with any of the above constructs may be modified to include additional genetic modifications and may, e.g., be hypoimmune, including e.g., hepatocytes made hypoimmune by disruption at an HLA class I locus (such as a B2M locus) and introduction of an NK cell decoy receptor transgene (such as e.g., a CD47, HLA-E, or B2M-HLA-E fusion transgene). Contacting the human hepatocytes with reagents to induce hypo-immunity (e.g., a B2M editing composition and a NK cell decoy receptor transgene) is performed before, during, or after transduction with the above identified expression construct.

Where transduction is performed prior to hepatocyte expansion, the transduced hepatocytes are transplanted into one or more recipient rodent bioreactors (such as e.g., an FRG rat, an FRGN mouse, or the like) via intrasplenic or portal vein injection and the rodent(s) is/are maintained under conditions sufficient for engraftment and expansion of the transplanted engineered hepatocytes. Following expansion in the bioreactor(s), the bioreactor liver(s) is/are harvested and perfused to retrieve the expanded population of engineered human hepatocytes. The retrieved engineered human hepatocytes are processed through enrichment, purification, and/or isolation procedures. The resulting processed cell population is subsequently prepared for delivery or cryopreserved for later delivery to a subject in need thereof.

Where transduction is performed following hepatocyte expansion, expanded hepatocytes are retrieved from one or more rodent bioreactors and transduced with one of the above identified constructs before or after further processing for enrichment, isolation, purification and/or isolation of the desired hepatocytes (with or without cryopreservation at any convenient point). The resulting transduced and processed cell population is subsequently prepared for delivery or cryopreserved for later delivery to a subject in need thereof.

A population of the prepared engineered hepatocytes are formulated into a dose formulation in a suitable delivery medium. The prepared dose formulation is delivered to a subject in need thereof through a medically appropriate route, such as e.g., via intrasplenic or portal vein injection or infusion, to treat the subject for the Factor IX deficiency and Hemophilia B.

### Example 6: Generation and Expansion of Factor VIII Engineered Human Hepatocytes for Hemophilia A

The feasibility of using an LVV approach to introduce an exogenous Factor VIII (F8) transgene into primary human hepatocytes to generate F8-engineered hepatocytes, e.g., expanded in an in vivo bioreactor before or after transgene transduction, was evaluated. As an initial test, primary human hepatocytes were transduced ex vivo with a commercially available LVV overexpressing human F8 (an unoptimized surrogate for corresponding clinical constructs), at various multiplicity of infections (MOI) and the cells were maintained in vitro. As a control, primary human hepatocytes not transduced (i.e., non-treated control, NTC) were maintained under the same in vitro culture conditions. Supernatants were collected from MOI 2 transduced samples, MOI 7 transduced samples, and NTC samples at culture days 4, 5, and 6 and F8 activity was measured using a commercially available kit (Chromogenix Coatest SP4 Factor VIII Kit; DiaPharma, West Chester, OH). Following supernatant collection on culture day 6, the cells were harvested and lysed and the F8 activity assay was also performed on the cell lysates.

The amount of F8 activity in both the MOI 2 and MOI 7 samples showed increasing activity across the day 4, 5, and 6 timepoints. In comparison, F8 activity in the NTC samples was at baseline at all three time points. By the day 6 timepoint, F8 activity measured in the MOI 7 supernatant samples was at least four times (4×) greater than the NTC baseline level. Importantly, detection of F8 activity shows that the exogenous F8 is expressed and secreted by the engineered cells. Human F8 activity was correspondingly high in the day 6 cell lysates. These data demonstrate the ability to generate engineered human hepatocytes that overexpress human F8 and display F8 activity that is substantially greater than corresponding non-engineered human hepatocytes, even using an unoptimized surrogate F8-LVV for transduction.

Having demonstrated the ability to generate F8 overexpressing human hepatocytes, the following improved expression constructs were designed for introduction into human hepatocytes to facilitate expression of the therapeutic transgene product by engineered hepatocytes transplanted into subjects in need thereof, such as e.g., human subjects having a Factor VIII deficiency such as Hemophilia A.

F8 expression constructs employed in this example include a suitable promoter, such as e.g., the MND promoter (SEQ ID NO:001), operably linked to an F8 coding sequence, such as e.g., a full-length F8 coding sequence (SEQ ID NO:012), encoding a full-length F8 polypeptide (SEQ ID NO:013), a B-domain-deleted F8 (i.e., BDDrFVIII) coding sequence (SEQ ID NO: 014), encoding an BDDrFVIII variant polypeptide (SEQ ID NO:015), a FVIII-Fc fusion protein (i.e., F8.Fc) coding sequence (SEQ ID NO:016), encoding an F8.Fc polypeptide (SEQ ID NO: 017), or the like, operably linked to a suitable 3′ sequence, including e.g., a polyA signal.

Useful constructs include those encoding multiple polypeptides, such as a F8 polypeptide and a von Willebrand Factor (vWF) polypeptide (such as e.g., a vWF Fc fusion (i.e., vWF.Fc SEQ ID NO:019 encoded by SEQ ID NO:018), including e.g., where such polypeptides are expressed from a F8 coding sequence operably linked to a vWF coding sequence via a 2A-self cleaving sequence, such as a furin and glycine-serine-glycine containing 2A sequence, such as e.g., a furin.GSG.T2A (SEQ ID NO:020) or a furin.GSG.P2A (SEQ ID NO:021). Where multiple polypeptides, such as vWF and F8, are employed the coding sequences are arranged in any order.

Useful expression cassette arrangements include, e.g.:
[MND promoter]-[F8 full-length]-[polyA],
[MND promoter]-[F8 (B domain deleted)]-[polyA],
[MND promoter]-[F8.Fc]-[Furin.GSG.T2A]-[VWF.Fc]-[polyA],
[MND promoter]-[VWF.Fc]-[Furin.GSG.T2A]-[F8.Fc]-[polyA], and the like.

As will be readily understood, in some instances, substitutions may be made in the above-described constructs including e.g., exchange of the described promoter for another appropriate promoter, exchange of the transgene coding sequence for another coding sequence encoding the same transgene or a variant of the transgene, exchange of the sequence 3′ of the transgene for another 3′ sequence (e.g., including an alternative polyA or other 3′ components), or the like. Expression constructs are introduced into a suitable lentiviral vector for transduction into hepatocytes.

Hepatocytes are prepared, expanded, and transduced essentially as described in Example 5, substituting the above constructs for those constructs described in Example 5.

A population of the prepared engineered hepatocytes are formulated into a dose formulation in a suitable delivery medium. The prepared dose formulation is delivered to a subject in need thereof through a medically appropriate route, such as e.g., via intrasplenic or portal vein injection or infusion, to treat the subject for the Factor VIII deficiency and Hemophilia A.

### Example 7: Generation and Expansion of Human Hepatocytes Engineered with Urea Cycle Genes for Urea Cycle Disorders (UCD)

The following expression constructs were designed for introduction into human hepatocytes to facilitate expression of the therapeutic transgene product (or multiple transgene products) by engineered hepatocytes transplanted into subjects in need thereof, such as e.g., human subjects having a UCD.

Expression constructs employed in this example include a suitable promoter, such as e.g., the MND promoter (SEQ ID NO:001), operably linked to one or more urea cycle genes, such as e.g., those urea cycle genes that are rate-limiting in the metabolism of nitrogen waste, operably linked to a suitable 3′ sequence, including e.g., a polyA signal.

Useful sequences encoding urea cycle genes including e.g.:
a Carbamoyl-phosphate synthase (CPS1) coding sequence, such as e.g., a codon-optimized CPS1 coding sequence (SEQ ID NO:022), encoding a CPS1 polypeptide (SEQ ID NO:023), a N-acetylglutamate synthase (NAGS) coding sequence, such as e.g., a codon-optimized NAGS coding sequence (SEQ ID NO:024), encoding a NAGS polypeptide (SEQ ID NO:025), a Ornithine transcarbamylase (OTC) coding sequence, such as e.g., a codon-optimized OTC coding sequence (SEQ ID NO:026), encoding a OTC polypeptide (SEQ ID NO:027), or the like.

Useful constructs include those encoding multiple polypeptides, such as e.g., CPS1 and NAGS, CPS1 and OTC, NAGS and OTC, or CPS1, NAGS, and OCT, including e.g., where such polypeptides are expressed from a first coding sequence operably linked to a second coding sequence via a 2A-self cleaving sequence, such as a furin and glycine-serine-glycine containing 2A sequence, such as e.g., a furin.GSG.T2A (SEQ ID NO:020) or a furin.GSG.P2A (SEQ ID NO: 021). Where multiple polypeptides, such as first urea cycle coding sequence encoding a first polypeptide and a second urea cycle coding sequence encoding a second polypeptide, are employed the coding sequences are arranged in any order.

Useful expression cassette arrangements include, e.g.:
[MND promoter]-[CPS1]-[polyA]
[MND promoter]-[NAGS]-[polyA]
[MND promoter]-[OTC]-[polyA]
[MND promoter]-[CPS1]-[poly A]-[Furin.GSG.T2A]-[OTC]-[polyA]
[MND promoter]-[OTC]-[polyA]-[Furin.GSG.T2A]-[CPS1]-[polyA]
[MND promoter]-[NAGS]-[polyA]-[Furin.GSG.T2A]-[CPS1]-[polyA]
[MND promoter]-[CPS1]-[polyA]-[Furin.GSG.T2A]-[NAGS]-[polyA]
[MND promoter]-[NAGS]-[polyA]-[Furin.GSG.T2A]-[CPS1]-[Furin.GSG.P2A]-[OTC]-[polyA]

As will be readily understood, in some instances, substitutions may be made in the above-described constructs including e.g., exchange of the described promoter for another appropriate promoter, exchange of the transgene coding sequence for another coding sequence encoding the same transgene or a variant of the transgene, exchange of the sequence 3′ of the transgene for another 3′ sequence (e.g., including an alternative polyA or other 3′ components), or the like. Expression constructs are introduced into a suitable lentiviral vector for transduction into hepatocytes.

Hepatocytes are prepared, expanded, and transduced essentially as described in Example 5, substituting the above constructs for those constructs described in Example 5.

A population of the prepared engineered hepatocytes are formulated into a dose formulation in a suitable delivery medium. The prepared dose formulation is delivered to a subject in need thereof through a medically appropriate route, such as e.g., via intrasplenic or portal vein injection or infusion, to treat the subject for the urea cycle disorder.

### Example 8: Generation and Expansion of GLA Gene Engineered Human Hepatocytes for Fabry Disease

The feasibility of using an LVV approach to introduce an exogenous alpha-galactosidase A (GLA) transgene into primary human hepatocytes to generate GLA-engineered hepatocytes, e.g., expanded in an in vivo bioreactor before or after transgene transduction, was evaluated. As an initial test, primary human hepatocytes were transduced ex vivo with a commercially available LVV overexpressing human GLA (an unoptimized surrogate for corresponding clinical constructs), at various multiplicity of infections (MOI) and the cells were maintained in vitro. As a control, primary human hepatocytes not transduced (i.e., non-treated control, NTC) were maintained under the same in vitro culture conditions. Cells were collected from MOI 2 transduced samples, MOI 12 transduced samples, and NTC samples at culture day 5, then lysed and homogenized. Alpha galactosidase (alpha-Gal) activity was measured using a commercially available assay (Abcam, Cambridge, UK) which employs a specific synthetic substrate that releases a fluorophore (which can be quantified at Ex/Em 360/445 nm) upon alpha-Gal cleavage. A positive control sample, included in the aforementioned kit, was also employed.

The amount of alpha-Gal activity measured in all MOI 2 and MOI 12 samples was at least five times (5×) greater than the highest level of activity observed in the NTC. Moreover, the alpha-Gal activity measured in some of transduced samples with the highest activity was ten times (10×) or greater than the highest activity observed in the positive control samples. Collectively, these data demonstrate the ability to generate engineered human hepatocytes that overexpress human GLA and display alpha-Gal activity that is substantially greater than corresponding non-engineered human hepatocytes, even using an unoptimized surrogate GLA-LVV for transduction.

Having demonstrated the ability to generate F8 overexpressing human hepatocytes, the following improved expression constructs were designed for introduction into human hepatocytes to facilitate expression of the therapeutic transgene product by engineered hepatocytes transplanted into subjects in need thereof, such as e.g., human subjects having a lysosomal storage disorder, such as Fabry Disease.

Expression constructs employed in this example include a suitable promoter, such as e.g., the MND promoter (SEQ ID NO:001), operably linked to an alpha-galactosidase A gene (GLA), such as e.g., a GLA (1) coding sequence (SEQ ID NO:028), encoding a GLA (1) polypeptide (SEQ ID NO:029) or a GLA (2) coding sequence (SEQ ID NO:030), encoding a GLA (2) polypeptide (SEQ ID NO:029) or the like, operably linked to a suitable 3′ sequence, including e.g., a polyA signal.

Useful expression cassette arrangements include, e.g.:
[MND promoter]-[GLA (1)]-[polyA]
[MND promoter]-[GLA (2)]-[polyA]

As will be readily understood, in some instances, substitutions may be made in the above-described constructs including e.g., exchange of the described promoter for another appropriate promoter, exchange of the transgene coding sequence for another coding sequence encoding the same transgene or a variant of the transgene, exchange of the sequence 3′ of the transgene for another 3′ sequence (e.g., including an alternative polyA or other 3′ components), or the like. Expression constructs are introduced into a suitable lentiviral vector for transduction into hepatocytes.

Hepatocytes are prepared, expanded, and transduced essentially as described in Example 5, substituting the above constructs for those constructs described in Example 5.

A population of the prepared engineered hepatocytes are formulated into a dose formulation in a suitable delivery medium. The prepared dose formulation is delivered to a subject in need thereof through a medically appropriate route, such as e.g., via intrasplenic or portal vein injection or infusion, to treat the subject for the lysosomal storage disorder and Fabry Disease.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of the present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A method of generating hypoimmunogenic hepatocytes or progenitors thereof, the method comprising:
contacting a cell population comprising human hepatocytes or progenitors thereof with an editing composition under conditions sufficient to generate a human leukocyte antigen (HLA) class I deficiency in the hepatocytes or progenitors thereof; and
contacting the cell population with a transgene encoding at least one NK cell decoy receptor under conditions sufficient for expression of the transgene by the hepatocytes or progenitors thereof,
thereby generating a population of hypoimmunogenic hepatocytes or progenitors thereof.

2. The method of claim 1, wherein the editing composition is a beta-2-microglobulin (B2M)-editing composition.

3. The method of claim 1 or claim 2, further comprising introducing the generated population of hypoimmunogenic hepatocytes or progenitors thereof into a bioreactor.

4. The method of claim 3, wherein the bioreactor is an in vivo bioreactor and the in vivo bioreactor is maintained under conditions sufficient to produce an expanded population of hypoimmunogenic hepatocytes, optionally wherein the in vivo bioreactor is a mouse, rat, or pig.

5. The method of any of the preceding claims, wherein the at least one NK cell decoy receptor comprises CD47, a B2M-HLA-E fusion, or a combination thereof.

6. A method of treating a subject for a condition, the method comprising:
administering to the subject an effective amount of hypoimmunogenic hepatocytes or progenitors, wherein the hypoimmunogenic hepatocytes or progenitors each comprise an HLA class I deficiency and a transgene encoding at least one NK cell decoy receptor, optionally wherein the condition is a liver condition.

7. The method of claim 6, wherein the hypoimmunogenic hepatocytes or progenitors thereof are generated according to the method of any of claims 1 to 5.

8. A non-human mammal comprising an engrafted cell population, the cell population comprising a plurality of hypoimmunogenic human hepatocytes or progenitors thereof, wherein each hepatocyte or progenitor of the plurality comprises an HLA class I deficiency and a transgene encoding at least one NK cell decoy receptor, optionally wherein the HLA class I deficiency comprises a B2M deficiency and the at least one NK cell decoy receptor comprises CD47, a B2M-HLA-E fusion, or a combination thereof.

9. A population of hepatocytes or progenitors thereof comprising an expanded population of hypoimmunogenic human hepatocytes or progenitors thereof isolated from the non-human mammal of claim 8.

10. A cell population comprising a plurality of hypoimmunogenic primary human hepatocytes, wherein each hepatocyte of the plurality comprises an HLA class I deficiency and a transgene encoding at least one NK cell decoy receptor, optionally wherein the HLA class I deficiency comprises a B2M deficiency and the at least one NK cell decoy receptor comprises CD47, a B2M-HLA-E fusion, or a combination thereof.

11. A method of generating genetically modified human hepatocytes, the method comprising:
contacting a cell population comprising human hepatocytes or progenitors thereof with an integrating vector comprising a transgene encoding a gene product under conditions sufficient for functional integration of the transgene to produce genetically modified hepatocytes or progenitors thereof comprising the integrated transgene; and
transplanting the genetically modified hepatocytes or progenitors thereof into an in vivo bioreactor and maintaining the in vivo bioreactor under conditions sufficient for expansion of the genetically modified hepatocytes or progenitors to generate an expanded population of genetically modified human hepatocytes that express the gene product, optionally wherein the in vivo bioreactor is a mouse, rat, or pig.

12. The method of claim 11, wherein the transgene encodes a gene product selected from the group consisting of: Copper-transporting ATPase 2 (ATP7B), Hereditary hemochromatosis protein (HFE), Hemojuvelin, Hepcidin (HAMP), Transferrin receptor protein 2 (TFR2), Solute carrier family 40 member 1 (SLC40A1), Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase (ARG), alpha-1 antitrypsin (AAT), fumarylacetoacetate hydrolase (FAH), Argininosuccinate lyase (ASL), Argininosuccinate synthase (ASS), Ornithine translocase (ORNT1), citrin, UDP-glucuronosyltransferase 1A1 (UGT1A1), Transthyretin (TTR), Serine—pyruvate aminotransferase (AGXT), Complement factor H (CFH), and combinations thereof.

13. A method of treating a subject for a condition, the method comprising:
administering to the subject an effective amount of genetically modified human hepatocytes generated according to the method of any of claim 11 or 12.

14. The method of claim 13, wherein the condition is a liver condition or a genetic disease, optionally wherein the genetic disease is a monogenic disease, optionally wherein the condition is: a Factor VIII deficiency and the transgene encodes Factor VIII; a Factor IX deficiency and the transgene encodes Factor IX; a urea cycle disorder (UCD) and the transgene encodes one or more urea cycle polypeptides; or a lysosomal storage disease and the transgene encodes an enzyme associated with the lysosomal storage disease.

15. A non-human mammal comprising an engrafted cell population, the cell population comprising a plurality of genetically modified human hepatocytes, wherein each hepatocyte of the plurality comprises a functionally integrated transgene encoding a gene product.

16. The non-human mammal of claim 15, wherein the engrafted cell population is an in vivo expanded cell population, and the non-human mammal further comprises hepatocyte progeny of the genetically modified human hepatocytes.

17. A population of hepatocytes or progenitors thereof comprising an expanded population of genetically modified human hepatocytes isolated from the non-human mammal of claim 15 or 16.

18. A cell population comprising a plurality of hypoimmunogenic primary human hepatocytes, wherein each hepatocyte of the plurality comprises an HLA class I deficiency and a transgene encoding at least one NK cell decoy receptor.

19. A method of generating a plurality of hepatocyte cell therapy doses, the method comprising:
(1a) genetically modifying human hepatocytes and expanding the genetically modified human hepatocytes in one or more in vivo bioreactors to generate an expanded population of genetically modified human hepatocytes, or
(1b) genetically modifying expanded human hepatocytes obtained from one or more in vivo bioreactors to generate an expanded population of genetically modified human hepatocytes; and
(2) aliquoting the expanded population of genetically modified human hepatocytes of 1a or 1b into a plurality of hepatocyte cell therapy doses.

20. A method of treating a plurality of subjects having a condition, the method comprising:
generating a plurality of hepatocyte cell therapy doses according to claim 19; and
administering one or more doses of the plurality to each of the subjects to treat the subjects for the condition, optionally wherein the human hepatocytes are derived from a single human liver.


SEQUENCE LISTING (47 sequences)

SEQ ID NO: 1 - DNA, 389 bp, Artificial sequence
  myeloproliferative sarcoma virus enhancer, negative control region deleted, dl587rev primer-binding site substituted
tgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcg

SEQ ID NO: 2 - DNA, 1386 bp, Artificial sequence
  F9 coding sequence encoding an F9 Padua variant polypeptide
atgcagagagtcaacatgattatggccgagtctcccgggctgatcacaatatgccttctcggctaccttttgtcagcagaatgcaccgtcttcttggatcatgaaaacgcgaataagatcctcaaccgacctaagaggtataattcaggtaaactcgaggagttcgttcagggaaatttggagagagaatgtatggaggagaaatgcagcttcgaggaagcgcgagaagtgtttgagaacactgaacggacaactgaattttggaagcaatatgtcgacggggatcagtgtgagagcaacccctgcctgaatggagggtcatgcaaggatgatattaacagctatgagtgttggtgtccttttgggttcgagggcaagaattgcgaattggatgttacatgtaacattaaaaatggtagatgcgagcagttttgcaagaacagcgcagataataaggttgtttgttcctgtacggaaggatatcgcctcgcagaaaaccagaaaagttgcgaaccggctgtacctttcccatgtggccgagtaagtgtctcccagactagtaaacttacccgagcggagactgtgttccccgatgtagactatgtcaactccacggaagcggaaacaatacttgataatattacacaatccacgcaatccttcaatgattttactcgcgttgtagggggcgaggatgccaaacccgggcaattcccttggcaagtggtcctgaacggtaaggttgacgctttttgcggcgggagtattgttaacgaaaagtggatcgtcaccgcagcgcattgtgttgaaaccggcgtcaagattaccgtggttgcaggggagcataatatcgaggaaacggagcatactgagcagaaaagaaacgttattagaattattccccatcacaattacaatgcagcgataaataagtataatcatgatatcgctctgctggaacttgacgaaccgttggtattgaacagttacgtcacccccatctgtatagcggacaaggagtacacgaacatctttctgaaatttggaagcggctacgtgagcgggtggggtagagtattccacaagggtcgatccgcccttgttctgcaatacctcagagttcctcttgtagatagggcaacctgtcttcttagtaccaagtttactatctataataacatgttctgtgccggatttcatgagggcgggagggattcatgccagggtgattctggagggcctcacgtcactgaggtggagggtacgagttttctgacaggaattatttcttggggagaagaatgcgcgatgaagggcaagtatggtatctacactaaagtgagtaggtatgtgaattggattaaagaaaagactaagttgacatga

SEQ ID NO: 3 - protein, 461 aa, Artificial sequence
  F9 Padua variant polypeptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 4 - DNA, 234 bp, Artificial sequence
  F9 3′ long terminal repeat
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 5 - DNA, 408 bp, Unknown
  spleen focus-forming virus promoter
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 6 - DNA, 1647 bp, Unknown
  Luc coding sequence encoding a Luc polypeptide
atggaagatgccaaaaacattaagaaaggcccagcgccattctacccactcgaagacgggaccgctggcgagcagctgcataaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgtagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctgagcgtggtgccatttcaccacggcttcggcatgttcaccacgctgggctacttgatctgcggctttcgggtcgtgctcatgtaccgcttcgaggaagagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcatagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgcc

SEQ ID NO: 7 - protein, 549 aa, Unknown
  Luc polypeptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIA

SEQ ID NO: 8 - DNA, 720 bp, Unknown
  EmGFP coding sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - protein, 239 aa, Unknown
  EmGFP polypeptide
MVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 10 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 11 - DNA, 1386 bp, Homo sapiens
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 12 - DNA, 7056 bp, Homo sapiens
atgcagatcgagctgtcaacatgcttcttcctctgcctgctgcggttctgtttcagcgccacccgcagatactacctgggcgccgtggagctgtcctgggattatatgcagtctgatctgggagaactcccggtggacgctagattcccacccagagtgcctaagagtttccccttcaacacaagcgtagtgtacaagaagaccctctttgtggagttcacagaccacctattcaacatcgctaagcctagaccaccttggatgggcctcctgggacctacaatccaagccgaggtgtacgacaccgtggtgatcaccctgaaaaacatggcttcgcaccctgtgtctctccatgccgtgggtgtgagctactggaaggccagcgagggcgctgaatacgacgaccagacatcccagagagagaaggaggacgataaggtgttcccaggtggatctcacacctacgtgtggcaggtgctgaaggagaacggccctatggcctccgaccctttatgcttgacctacagctacctgagccacgtggacctggtgaaagacctcaactccggcctgatcggtgccctgctggtgtgtagagaaggatctcttgctaaggagaagacccagaccctgcacaaattcatcctgctgttcgccgtgtttgacgaggggaaaagctggcacagcgagacaaaaaattccctgatgcaggacagagacgccgcttctgcccgcgcctggccaaagatgcacaccgtcaatggctacgtcaatagaagcctgcccggcctgatcggctgtcacaggaaatccgtgtactggcacgtgataggcatgggcactacccctgaagtgcatagcattttcctggaggggcacacttttctggtgcggaatcaccggcaggcctctctggagatctctcctatcacatttctgaccgctcagacactgctcatggatctcggccagttcctgctgttctgccacatcagctcccaccagcacgacggtatggaggcctacgtgaaagtggacagctgccctgaggaacctcaactgcggatgaagaacaacgaggaggccgaggactacgatgacgacctgaccgatagcgagatggacgtggtcagatttgacgacgacaatagccctagcttcatccagatcagaagcgtcgccaagaagcaccccaagacctgggtccactacatcgccgctgaagaggaggattgggactacgcccccctggtgctggcccctgacgacagatcttacaagtcccagtacctaaacaatggccctcagagaatcggcagaaagtacaagaaagtgcgcttcatggcctatacagacgagaccttcaaaaccagagaagctatccaacacgagtctggcatcctgggcccgctgctgtacggcgaggtgggcgatacactcctgattatcttcaagaaccaggcctctcgcccttacaacatctaccctcacggcatcacagatgtgcggcctctgtactccagacggctgcccaagggcgtgaagcacctgaaagacttccccatcctgcccggagagatcttcaaatacaaatggaccgtcaccgtggaagatggacctaccaaaagcgaccccagatgcctgaccagatactactcctctttcgtgaacatggaacgggacctggcctcaggcctaattggcccactgctgatctgctacaaggagagcgtggaccaacgggggaaccagattatgagcgataagagaaatgtgatcctgttctctgtgtttgacgagaaccggagctggtacctgacagagaacatccaaagattcctgccgaatccggccggcgtccagctggaggacccagagttccaggcctccaacattatgcactcaatcaacggctacgtgtttgattcactgcaactgtcagtgtgtctgcacgaggtggcctactggtacatcctgtcaataggagcccaaaccgattttctgagcgtgtttttctccggctacaccttcaagcacaagatggtttacgaggataccctgacactgttcccatttagcggcgaaactgtgtttatgagcatggaaaaccccggactgtggatcctgggctgtcacaattctgactttagaaacagaggaatgaccgcactgctgaaggtgagctcttgcgacaagaacacaggagactactacgaagacagctacgaggatatttctgcctatctgctctctaaaaacaacgccattgaaccccggtcttttagccaaaattctagacaccctagcacgagacagaagcagttcaacgccacaaccatccccgagaatgatatcgagaaaaccgacccctggttcgcgcacagaacccctatgcctaagatccaaaacgtgtcctctagcgatctgctgatgctgctgcgccagagcccaactcctcacggcctgagcctgtccgacctgcaggaggccaagtacgaaaccttctccgacgatccttctccaggagctatcgacagtaacaactcgctgtctgagatgacccactttagaccccagttgcatcacagcggcgatatggtgtttacaccggagtcgggccttcaactacgcctcaacgaaaagctgggaaccactgctgcaactgagctgaaaaagctggacttcaaggtcagtagcaccagcaataaccttatctcgactatccctagcgacaatctggctgccggcaccgacaacacctcgtctctgggccctcccagcatgcccgtccattacgattcacagctggatacaaccctcttcggcaagaagagcagccctctgaccgaaagcggcgggcccctgtcgctgagcgaggagaacaacgatagtaagctgcttgaaagcggcctcatgaacagccaggagagcagctgggggaaaaatgtgtctagcaccgaatccggaagactgttcaagggcaagcgggctcacggccctgcactgctgacaaaggacaacgccctgttcaaggtcagtatctctctcttgaagacaaacaaaacaagcaacaactccgccaccaacaggaagacgcacatcgacgggcccagtctgctgatcgagaacagcccttccgtatggcagaacattttagaatcagacaccgaattcaagaaggtgacccctctgatccatgatagaatgctgatggacaagaacgccaccgccttgagactgaaccacatgagcaacaagactaccagcagcaagaacatggaaatggtgcagcagaagaaggagggtccgatccctccggatgcccagaaccctgatatgagcttcttcaagatgctattcctgcctgagtctgctaggtggatccagcggacccacggaaagaactctctgaatagcggccagggcccttcccctaaacagctcgtcagcttaggccctgaaaagtccgtggagggccagaatttcctctcagaaaagaacaaggtggttgtgggaaaaggcgagttcaccaaggacgtgggcctgaaggaaatggtgttccccagctctagaaacttgtttctgacgaacctggacaacctgcacgagaacaacacccacaaccaggaaaaaaagatccaggaggagatcgagaaaaaggaaacactgatccaggagaacgtggtgctgccccagatccacacggtcacagggaccaagaatttcatgaagaacctgttcctcctgtcaacccggcagaacgtggagggtagctacgacggcgcgtacgctcccgtgcttcaggacttcagaagcctgaacgactctacaaaccggacaaagaagcacaccgcacacttctctaaaaagggtgaggaagagaacctggagggcttaggcaaccagacaaagcagatcgtggagaagtacgcctgtaccaccagaataagcccaaacacatcccaacagaatttcgtgacccagagaagtaagcgcgcgctgaaacagttccggctacccctggaagagaccgaactggaaaagagaatcatcgtggacgacacaagcacccagtggagcaagaatatgaaacacttgactccatctacgttgacccagatcgactacaacgagaaggagaagggagccatcacccagagccctctgtctgactgcttaactcggagccacagcatcccccaggccaacagatctcccctgcccatcgccaaagtaagcagcttcccttctatcagacctatctatctgaccagagtgctgtttcaggacaatagcagccatctgcctgccgctagctacagaaagaaggattccggtgtgcaggaaagcagccacttcctccagggcgccaagaaaaataatctgtctctggcgatcctgacgctggagatgaccggtgaccagcgggaggtgggcagcctgggaaccagcgctacaaactctgtgacatacaagaaggtggaaaacaccgtgctgcccaaacctgacctgcctaagaccagcggaaaggttgagcttctgcccaaggtgcacatctaccagaaagacctgttccccaccgagaccagcaacggcagcccgggccacctggacctcgtggaaggttctctgctgcagggcaccgaaggcgccatcaagtggaacgaggccaatcggcctggaaaagtaccattcctgagagtggccaccgaatcttcagccaagactccaagcaaactgttggatcctttggcctgggacaaccactacggcacacagattcccaaggaggagtggaagagccaggaaaaaagccctgagaagaccgccttcaagaagaaggacaccatcctgtctctgaacgcctgcgagagcaaccacgccatcgccgctattaatgaaggccagaacaagcctgaaatcgaggtcacctgggccaagcagggccggacagaaagactgtgttctcaaaaccctccagttctcaagcggcaccagcgggaaattacccgaacaaccctgcaatctgaccaggaagaaatcgactacgacgatacaatctctgttgagatgaagaaagaggactttgatatctacgacgaagatgagaaccagagcccaagatcctttcaaaagaagaccagacactatttcatcgcggccgtggagaggctgtgggattacggcatgagctccagtccccatgtgctgcggaacagagcccagagcggctcggtcccgcaatttaagaaggtagtgttccaggagtttaccgacggcagttttacccaacctctgtatcggggcgagctgaacgaacacctgggcctcctgggcccgtacatcagagccgaggttgaggataacatcatggtgaccttccggaaccaggcgtccagaccttattcattttactccagcctgatctcgtatgaggaagaccagaggcagggcgctgagcctagaaaaaatttcgtgaagccgaacgagacgaagacctacttctggaaagtgcagcaccacatggcccccaccaaggacgagttcgattgcaaggcctgggcctacttctccgacgtggacctggagaaggatgtgcactctggcctgatcggccctctgctggtttgccacaccaacacattaaaccctgcccatggcagacaggtaaccgtccaggagttcgcgctgttttttaccatctttgacgaaaccaagtcctggtactttacagagaatatggaaagaaactgcagagccccatgcaacatccagatggaagatccaacctttaaggagaactacagattccacgccattaacggatacatcatggacaccctgccgggccttgtgatggcccaggaccagcggatccggtggtacctgctgtctatgggcagcaacgagaatatccacagcatccacttctctggccacgtgttcacagtgcggaaaaaggaagagtataagatggccctgtacaatctataccccggcgtgttcgagaccgtggagatgctgcctagcaaggccggtatctggagagtagagtgcctgatcggcgagcacctgcacgccggaatgagcacactttttctcgtgtactccaacaagtgccagacccctctgggcatggccagcggtcatatcagagattttcagatcaccgcaagcggccagtacggacagtgggctccaaagctggccagactgcactacagcggttctatcaacgcctggtccacaaaggaacctttcagttggatcaaggtggacttgctggcccctatgatcatccacggcatcaagacccagggcgccagacagaagttctctagcctgtacatcagccagttcatcataatgtactctctggacggcaaaaagtggcagacataccggggcaactctaccggcaccctgatggtgttcttcggcaacgtagactcttcgggcattaaacacaacatcttcaatccacctatcatagcccggtacatcagattacaccctacacactatagcatccggtctacactgagaatggaactgatgggctgcgacctgaactcatgcagcatgcctctgggcatggaatcaaaagctataagcgacgcccagatcaccgcctcgtcctacttcacaaatatgttcgccacatggtcaccttccaaggcacgactgcacctgcagggccggtctaacgcttggcgccctcaggtgaacaatcctaaggaatggctgcaagtggatttccaaaagacaatgaaggtcactggcgttacaacacagggggtgaagagcctgctgacaagcatgtacgtcaaggaattcctgatcagcagcagccaggacggccaccagtggaccctgttcttccagaacggcaaggtgaaagtgtttcagggaaaccaagacagcttcactccagtggtgaacagcttagaccctcccctactgaccagatacctgaggatccacccccagagctgggtgcaccagatcgccctgcggatggaagtgctgggctgcgaggcccaggacctctattga

SEQ ID NO: 13 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 14 - DNA, 4374 bp, Artificial sequence
  B-domain-deleted F8 coding sequence
atgcagattgagctgtctacttgtttttttctgtgcctgctgaggttctgcttctctgccaccaggaggtattacctgggggctgtggagctgagctgggattacatgcagtctgatctgggggagctgcctgtggatgccaggttcccccccagggtgcccaagagcttccccttcaacacctctgtggtgtataagaagaccctgtttgtggagttcactgatcatctgtttaacattgccaagcccaggcccccctggatgggcctgctgggcccaactatccaggctgaggtgtatgacactgtggtcatcaccctgaagaatatggccagccatcctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacttatagctacctgtctcatgtggacctggtgaaggacctgaactctggcctgattggggccctgctggtctgtagggaaggcagcctggccaaggagaagacccagaccctgcacaagtttattctgctgtttgctgtgtttgatgaaggcaagagctggcactctgagaccaagaattctctgatgcaggatagggatgctgcctctgccagggcctggcccaagatgcatactgtgaatggctatgtgaacagaagcctgcctggcctgattggctgccataggaagtctgtgtattggcatgtgattgggatgggcactacccctgaagtgcacagcattttcctggagggccacactttcctggtgaggaaccacaggcaggcctctctggagatcagccccattactttcctgactgcccagaccctgctgatggatctgggccagttcctgctgttctgccacatctctagccaccagcatgatggcatggaggcctatgtgaaggtggacagctgccctgaggagccccagctgaggatgaagaataatgaggaggctgaggattatgatgatgacctgactgactctgagatggatgtggtgaggtttgatgatgataatagccccagcttcatccagatcaggtctgtggccaagaagcatcccaagacctgggtgcactatattgctgctgaagaggaggactgggactatgcccctctggtgctggctcctgatgacaggagctataagagccagtatctgaacaatgggccccagaggattgggaggaagtacaagaaggtgaggttcatggcctacactgatgagacctttaagaccagggaggccatccagcatgagtctggcattctggggcccctgctgtatggggaggtgggggacactctgctgatcattttcaagaaccaggccagcaggccctacaatatttacccccatggcatcactgatgtgaggcccctgtacagcaggaggctgcccaagggggtgaagcacctgaaggacttccccatcctgcctggggagatcttcaagtacaagtggactgtgactgtggaggatggccctaccaagtctgaccctaggtgtctgactaggtactacagcagctttgtgaacatggagagagacctggcttctggcctgattggccccctgctgatctgctacaaggagtctgtggatcagaggggcaaccagattatgtctgataagaggaatgtcatcctgttctctgtgtttgatgagaacaggagctggtatctgactgagaacattcagaggttcctgcccaaccctgctggggtgcagctggaggaccctgagttccaggccagcaacatcatgcattctattaatggctatgtgtttgacagcctgcagctgtctgtgtgcctgcatgaggtggcctactggtacatcctgagcattggggcccagactgactttctgtctgtgtttttctctgggtacaccttcaagcacaagatggtctatgaggacaccctgaccctgttccccttttctggggaaactgtgtttatgagcatggagaaccctgggctgtggatcctgggctgccacaactctgactttaggaataggggcatgactgccctgctgaaggtgagcagctgtgacaagaatactggggattactatgaggacagctatgaggatatctctgcctacctgctgagcaagaacaatgccattgagcctaggagcttcagccagaacccccctgtgctgaagaggcaccagagggagatcaccaggaccaccctgcagtctgatcaggaggagattgactatgatgacaccatctctgtggagatgaagaaggaggactttgatatttatgatgaggatgagaaccagagccccaggagcttccagaagaagaccaggcactatttcattgctgctgtggagaggctgtgggactatggcatgagctctagcccccatgtgctgaggaacagggcccagtctggctctgtgccccagttcaagaaggtggtgttccaggaatttactgatggcagctttacccagcccctgtacagaggggagctgaatgagcacctgggcctgctgggcccctacatcagggctgaggtggaggataatatcatggtgacctttaggaaccaggcctctaggccctattctttttacagcagcctgatcagctatgaggaggaccagaggcagggggctgagcctaggaagaactttgtgaagcccaatgagaccaagacctacttttggaaagtgcagcaccacatggcccccactaaggatgagtttgattgcaaggcctgggcctatttctctgatgtggacctggagaaggatgtgcactctggcctgattggccccctgctggtgtgccacaccaacactctgaaccctgcccatggcaggcaggtgactgtgcaggagtttgccctgttctttaccatctttgatgagactaagagctggtatttcactgagaacatggagaggaactgcagagccccttgcaacatccagatggaggaccctaccttcaaggagaactataggttccatgccatcaatgggtacatcatggataccctgcctggcctggtgatggctcaggaccagaggatcaggtggtacctgctgagcatggggagcaatgagaacattcatagcatccacttctctgggcatgtgttcactgtgaggaagaaggaggagtataagatggccctgtacaacctgtaccctggggtgtttgagactgtggagatgctgcccagcaaggctggcatctggagggtggagtgcctgattggggagcacctgcatgctggcatgagcactctgttcctggtgtacagcaacaagtgccagacccccctgggcatggcctctggccacatcagggacttccagattactgcctctgggcagtatgggcagtgggcccccaagctggccaggctgcactactctgggtctatcaatgcttggagcaccaaggagcctttcagctggatcaaggtggatctgctggcccccatgatcattcatgggatcaagacccagggggccaggcagaagttcagcagcctgtatatttctcagttcatcatcatgtattctctggatggcaaaaagtggcagacctatagagggaacagcactgggaccctgatggtgttttttggcaatgtggatagctctggcatcaagcacaatatcttcaacccccccattattgccaggtacatcaggctgcaccccacccactactctatcaggagcaccctgaggatggagctgatgggctgtgatctgaacagctgctctatgcctctggggatggaaagcaaggccatctctgatgcccagatcactgccagcagctatttcaccaatatgtttgccacttggagccctagcaaggctaggctgcatctgcagggcaggtctaatgcctggaggccccaggtgaacaaccccaaggagtggctgcaggtggacttccagaagactatgaaagtgactggggtgaccacccagggggtgaaaagcctgctgaccagcatgtatgtgaaggagttcctgattagcagcagccaggatggccaccagtggaccctgttcttccagaatgggaaggtgaaggtgtttcagggcaatcaggatagcttcaccccagtggtgaacagcctggacccccccctgctgaccaggtacctgaggatccacccccagagctgggtgcaccagattgccctgaggatggaggtgctgggctgtgaggcccaggatctgtactga

SEQ ID NO: 15 - protein, 1457 aa, Artificial sequence
  B-domain-deleted F8 variant polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - DNA, 5829 bp, Artificial sequence
  FVIII-Fc fusion protein coding sequence
gccaccaggagatattaccttggcgccgtggaacttagttgggattacatgcagtctgacctgggcgagctcccggtcgacgccagatttcctccacgtgtgccaaaatcttttccatttaacacgagcgttgtgtataaaaagaccctgttcgtggaattcacagaccatctgttcaacattgctaagcccaggcctccctggatggggctcttgggccccaccatccaggcggaggtgtacgacacagtcgtgattactctgaagaacatggcatcacacccggtgtccttgcacgcggtgggcgtctcttactggaaggcatccgagggtgccgagtacgacgatcagacttcccagcgtgagaaggaggacgataaggtgttccccggcggtagccatacgtacgtgtggcaagtgctgaaggagaacggcccaatggcgagcgaccccctgtgcttgacgtactcttacctgtctcacgtggatctggtcaaagacctgaactccggcttgatcggtgctctcctggtgtgtcgggagggatccctggctaaggaaaaaacccaaaccctgcataaattcattctgctttttgccgtctttgacgagggcaagagctggcactccgagacaaagaacagtctgatgcaggatagagacgcagcctccgcccgcgcctggccgaaaatgcataccgtcaacgggtacgtgaacagatccctgccgggcctgattggctgccataggaagtcagtgtactggcatgtcatcggaatggggaccactcccgaggttcacagcattttcctggaaggtcacaccttcctggtcaggaaccatcgccaggccagccttgagatcagccccattaccttcctgacggcccagacgttgctcatggacctgggacaattccttctgttctgtcacatcagcagtcatcagcacgatggaatggaagcctacgtgaaggtggactcttgccccgaagagccgcaactgcgcatgaaaaataacgaggaagctgaggactatgatgacgatctgactgacagcgaaatggacgtggtccgcttcgacgatgacaactccccttctttcatccagatccgctccgtggcgaagaaacaccccaagacctgggttcactacatcgccgcagaagaggaagactgggattacgcaccccttgtgctggccccagacgatcgctcctacaaatcccagtacctgaataacggaccacaaagaattggcagaaagtataagaaagtgcgcttcatggcatataccgacgaaactttcaagacccgcgaggctattcagcacgagagcggcatcctcggtccacttctgtatggtgaggtgggggatactctgcttattatcttcaagaatcaggcttccaggccatataacatctatcctcacggaatcacagatgtcaggccgctgtattcccgcagactgcccaagggggtgaagcatctgaaggatttcccgattcttcccggtgagatcttcaagtataagtggaccgtgacagttgaagatggccccacaaagtccgatcctagatgcctgactcgctactattcaagctttgtgaacatggagcgcgatctggcctccggcctgatcggccctttgcttatctgttacaaggagtctgtcgatcagcgggggaaccagatcatgtccgataaaaggaatgtcatcctgttcagcgtgttcgacgaaaatcgtagctggtacctgactgagaatatccagcgttttctgccgaaccctgccggcgtgcaactggaggatccagagttccaggcttctaacattatgcactccatcaacggttacgtcttcgactctttgcaactgtccgtgtgcctgcatgaggtggcgtactggtatatcctctccatcggcgcccaaaccgacttcctgagcgtgtttttctccggctacaccttcaagcacaaaatggtgtacgaggacaccctcaccctgtttcctttcagcggggagactgtcttcatgagcatggaaaacccgggcttgtggatcctgggctgccacaactctgatttccgcaaccgcggcatgactgctctccttaaagtgtctagctgtgataagaacaccggtgactattacgaagatagctacgaggatatctccgcctacctgctctccaagaataacgccatcgagccccggtcattctcccagaatggaacgtccgagtccgccaccccagaatccggcccaggtagcgagccagcaactagtggcagcgagactcccggaactagcgagtccgctacccccgagagcggccccggatcagagccagccacctccggttctgaaaccccaggcacctctgagagcgccactcccgagtccggccccggaacgtccaccgaaccttctgaaggctccgctccaggttcacccgctggatctcctacttccaccgaggaagggaccagtgagtctgcgactcccgaatccggaccgggatctgagccagcgacctctggctctgaaacacccggcacctccgagtccgcaacacctgagtctggccccggctctccagccggctccccaacttccaccgaagagggctcccctgccggctcccctacgtccacggaagagggaacctctacggagccgtccgagggctccgccccgggtacaagtgagtccgccacaccagaatctgggcccggcacctccgaatccgctaccccagagagcggccctggtacaagcgagtccgcgacccctgaatctggcccgggatccgagccagccaccagcggatctgagacacctgggtctgaacccgccaccagcggttccgaaacgcctgggtcccccgccggatctcccacatccaccgaagagggaacaagtactgagcctagcgagggctccgctcctggtacctccactgagcccagtgagggtagcgctccgggctctgagcccgccacttcagggtcagagactcccggtacttctgaatccgccacccctgagtctggccccggcacctctactgaaccaagcgaaggctcagcacccgcgagttccgaaatcacacgcaccactctgcaaagcgatcaggaggaaattgactacgatgacaccatctccgtcgagatgaaaaaggaagatttcgacatttatgatgaagacgagaatcagagcccccgttcctttcagaaaaagacgcgccactacttcatcgcagctgtggagagactctgggactacggtatgtcctctagtccccatgtcctccgtaaccgtgcccaatcaggcagcgtgccccagtttaaaaaggtcgtgttccaggagttcaccgacgggtctttcacacagccgctctacaggggcgagctgaacgagcacctcggcctgctcggcccctacatccgggcggaagtggaggataacattatggtgacttttcgcaatcaggcctcccgtccctacagcttttacagttccctcatctcctacgaggaagatcagcgccaaggtgctgaacctcgcaagaacttcgtgaagcctaacgagacaaaaacctatttctggaaggtccagcaccatatggcccctaccaaggacgagttcgactgtaaagcatgggcgtacttttctgacgtggacctggagaaggatgtccactccggattgatcggtccgcttctcgtgtgccataccaatacactgaacccagcacacggccgccaggtgaccgtgcaagagtttgctctgtttttcaccatctttgacgagaccaagtcctggtactttactgaaaacatggagagaaactgtcgtgctccctgcaacattcagatggaggacccgacctttaaggagaattaccgtttccatgccatcaacggctatatcatggatacccttcccggcctcgtgatggcccaggatcagagaatccgctggtatcttctgtctatgggcagcaacgagaatatccattctattcacttctctggccatgtttttaccgtcaggaaaaaggaagagtataagatggccctgtataacctttaccccggagtgtttgagactgtcgagatgctgccaagcaaggccggaatctggcgcgtcgagtgtctcatcggcgagcacctgcatgctggcatgtctaccttgtttctggtgtacagcaacaagtgccagaccccgctggggatggcttccggtcacatccgcgacttccagatcactgccagcgggcagtacggacagtgggcgcctaagctggcgcgcctgcattactccggcagcattaacgcctggtccacaaaagaacccttctcttggatcaaagttgaccttctggcacctatgatcattcatggaatcaagacgcagggagccagacagaaattctcctctttgtacatttcacagttcattatcatgtatagcctggatggcaaaaagtggcagacttacagaggcaacagcaccggcaccctgatggtcttctttggtaacgtggactcttccggtatcaaacacaacatttttaatccacctattatcgcaaggtatattcgcctgcatcctactcactactccatccgttctaccctgaggatggaactcatgggatgtgatctcaactcatgctctatgcccctcggcatggagtccaaggctatttccgacgcgcagattacagcatccagctacttcaccaacatgttcgccacctggtctcccagcaaggctcgcctgcatctccaagggaggagtaacgcttggaggcctcaggtgaataaccctaaagagtggctgcaggtcgatttccaaaagactatgaaggtgaccggcgtaacaactcagggcgtgaaatctctgctcacctccatgtacgtgaaggagtttctgatctccagcagtcaggatggccaccagtggaccctttttttccagaatggcaaagttaaggttttccagggaaaccaagattcatttacaccggttgtgaactccctcgaccctcccctcctgacccgctatctgcgtatccatcctcagtcctgggtgcaccagatcgccttgcgcatggaggtgctgggctgcgaggcgcaggacctgtacgacaaaacccacacttgccctccctgtcccgctcctgagctgcttggcgggccgagcgtgtttctcttcccgcccaagccaaaggatacactcatgatctctcgcacccctgaggtcacctgtgtagttgtggatgtgagccacgaggaccccgaggtgaagtttaactggtacgtggacggcgtcgaggtccacaacgcgaagaccaaacctcgggaagagcaatataatagcacctatcgggtggttagcgtactgactgtgctgcaccaagactggttgaatggcaaggagtacaaatgcaaggtatcaaataaggctctccccgctcctatcgagaagaccatctccaaggccaaaggccagcctagagagccgcaggtgtacacgttgcccccttctagggacgagctgacaaagaaccaggtttcactgacatgcctggtgaagggtttttatccgagcgatatcgcagtggaatgggagagcaacggccagcctgagaataactacaagactaccccgcccgtcctggattctgatggaagttttttcctctattccaagctcactgtagataagtcccgttggcaacagggcaacgtcttttcttgcagcgtaatgcacgaggcgctccacaatcactatacccagaagagcctgagtctc

SEQ ID NO: 17 - protein, 1943 aa, Artificial sequence
  FVIII-Fc fusion polypeptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 18 - DNA, 4905 bp, Artificial sequence
  von Willebrand Factor-Fc fusion coding sequence
atgattcctgcacgctttgctggcgtgctcctggccttggcgctgattctgcctggcaccctgtgtgcagagggcacccgtggcagatctagcactgcgaggtgcagtctgttcggaagtgactttgtcaacaccttcgatggatctatgtattccttcgcaggctactgttcttatctcctggccggtggctgccagaagaggtccttcagcattatcggcgattttcagaacggtaagcgggtgtccctctctgtctacctgggcgaattctttgatatccacctgtttgtcaacggtactgtcactcagggcgatcagcgtgtgtcaatgccatacgctagcaaaggtctgtacctggagaccgaggccggctactataagctgtctggcgaggcctacggctttgtcgcccgcatcgacggttccggcaacttccaggtcttgctgtctgatcgttacttcaacaaaacctgcggtctgtgtggcaactttaacatcttcgctgaagacgatttcatgacacaggagggcacccttacctccgacccttacgacttcgccaattcttgggcactttcttccggcgagcaatggtgcgaacgcgcttctcctccaagtagctcctgtaacatcagctccggtgagatgcaaaagggcctgtgggagcagtgtcagctgttgaagtctacatcagtgttcgctcgctgtcaccctctggtcgatcccgagccatttgtggcactctgcgagaaaactctttgcgagtgtgccggtggcctggagtgcgcttgccctgcccttctggagtacgctcgcacctgcgcccaggaaggtatggtcctgtacggctggacagaccacagtgcgtgttcccccgtgtgccctgccggcatggagtatcgccagtgtgtgtccccgtgcgctagaacttgccagtcactgcatatcaacgagatgtgtcaggagaggtgcgtggacggctgctcttgccctgagggacagctcctggatgagggcttgtgcgtggaatccactgagtgcccttgcgtgcacagtggcaaaagataccctcccgggactagcttgtccagagactgcaacacttgtatctgtcgcaactcacaatggatttgctctaacgaagagtgccctggcgagtgcttggttaccggtcagagccactttaagagcttcgacaatcgctatttcaccttttccggcatctgtcaatacttgctggcccgtgactgtcaggaccactccttcagcattgtgatcgaaacggtgcagtgtgctgacgatcgtgatgccgtgtgcacccgcagtgtgaccgttcgcctccccggcctgcacaacagcctggtgaagctgaagcacggcgcgggcgtcgccatggacggccaagatattcaactgcctcttctgaagggcgacctgagaattcagcatacggtgactgcgagcgtgcgtttgtcctacggcgaggatctgcaaatggactgggacggtcgcggcaggttgctggtcaagctgtctccggtctatgcgggcaaaacctgcgggctgtgcggcaactacaatggtaatcagggcgatgacttcctcactcctagcgggctggctgaacctcgggtcgaggacttcggcaacgcgtggaagttgcacggagactgccaggacctgcagaagcaacactccgacccatgcgctctgaacccccgcatgactcgcttcagcgaagaggcctgtgcagtcctcacctcccccaccttcgaggcttgtcatcgtgccgtgagcccactcccatatctcaggaactgccggtacgatgtctgctcttgcagcgacggcagggaatgtctgtgcggtgcactggccagctacgcggccgcttgtgccgggagaggagtccgcgtcgcctggcgggagccaggtcgctgtgagctcaactgccccaaggggcaggtgtaccttcagtgtggcactccgtgcaatctgacctgtcgctccctttcttatcccgatgaagagtgtaacgaggcttgcctggagggctgcttttgcccgcctggactttacatggacgaacgtggtgactgcgtgccgaaggcccagtgtccgtgttattacgacggcgagattttccagcctgaggatattttctccgaccatcacaccatgtgttactgcgaggatggctttatgcactgtactatgagtggagtgcccggcagtctgttgccagacgcggtgctgagctcccccttgagtcatcggagcaagaggagcctgtcctgcaggcccccaatggttaagctggtgtgccctgccgataacctgcgcgccgaaggcctggagtgcactaagacttgtcagaattatgacctcgagtgtatgtccatgggttgcgtgagcggctgcctgtgccctcccggtatggtcagacacgagaatcggtgtgtcgctctggaacgctgtccgtgtttccaccaaggcaaggagtacgcccctggcgaaactgtgaaaatcgggtgcaacacttgtgtgtgtcgtgaccgcaagtggaactgcaccgaccacgtgtgtgacgcgacctgtagcaccattggaatggcccattacctgacctttgacggactgaagtatctgttccccggcgagtgtcagtatgttctggtacaggattactgtggtagtaaccccggcacattccggatcctggttgggaacaagggttgtagtcacccttctgtcaagtgcaaaaagcgtgttacgattctggtggaaggcggtgaaattgagcttttcgatggtgaggtgaatgtgaagcgccccatgaaggatgagactcatttcgaggtggtcgagtccggcaggtacattatcctgctcctggggaaggcactcagcgttgtatgggaccgtcatctgagcatctccgtcgtgctcaagcaaacataccaggaaaaggtgtgtggtctgtgtgggaactttgatgggatccagaacaatgaccttacttcctctaacctgcaggtggaggaagacccggtcgacttcgggaactcttggaaagtgagctcccagtgcgctgacactcggaaggtccccttggactcttcacccgctacatgccataataacattatgaagcagacaatggtggactcttcctgccgtattctgacctccgatgtatttcaggactgcaacaagctggtggaccccgagccttacttggatgtgtgtatctatgacacgtgcagttgcgaaagcatcggcgattgtgctgcgttctgtgacacaatcgccgcttacgctcacgtctgcgcccaacatggcaaggtagtgacttggcgtaccgccaccctgtgccctcagagttgtgaagagcgcaacctgagagagaacggatatgaagccgagtggagatacaactcttgcgcaccggcctgtcaggtgacttgtcagcatcccgagcccctcgcttgcccagtgcaatgtgtcgagggctgtcacgcgcattgcccgccagggaagatcctggatgagctgctccagacgtgtgtggatcctgaggactgtccagtctgtgaggtggccggccggcgcttcgccagtggtaaaaaggtcaccctcaatccttctgacccagaacactgccagatctgtcactgtgacgtagtgaacttgacctgtgaggcctgtcaagagcctacgagtgagtcagccactccggagtcaggacccggctcagagccagctaccagcggcagcgagacccccggaacatctgagtccgcaacacctgaaagtggtcctggatccgagcccgctacctccggctccgagaccccagggacaagcgagagtgccactcccgaatccggtcccgggacaagcaccgagccctctgagggttccgctccgggttccccggccggctcccctacctctaccgaagagggcacttctgagagtgcaacccccgaaagtggccccgggtctgagcctgcaacctctggctccgagactcctggtacaagcgagagcgccacccccgaaagcggcccagggagcccagctggcagcccgacctctaccgaggaagggagtccagctggttctcccacctcaaccgaagagggcatctctggcggagggggctctggagggggtggcagcctggtcccacgtggctctgggggaggcggatcagacaagacccacacttgcccaccctgtccggctcccgaattgctgggcggtccctccgtctttctgtttccccctaagcccaaggacacactcatgatctcccgcaccccggaggtgacctgtgtcgttgtcgacgtcagccatgaagaccccgaggtgaagttcaattggtacgttgacggggtggaggtccataacgcgaagaccaagccgcgcgaggaacagtacaacagcacataccgcgtcgtgtccgtgctcaccgtgctgcatcaggactggctgaacgggaaggaatacaaatgcaaggtatccaacaaagcgcttcccgccccgattgagaagacgattagcaaggccaagggccaaccaagggagccccaggtgtatactctgcccccttcccgtgacgaacttactaagaaccaggtttccctgacctgtttggtcaaaggtttctacccatccgacatcgctgtggagtgggaatcaaacggccagcccgaaaataactacaaaacgaccccgcctgtgcttgacagtgatggttcctttttcctgtacagcaagctgaccgtcgacaaatccaggtggcaacagggaaacgtcttctcctgtagcgtaatgcatgaggcactgcacaatcattatacccaaaagagcttgtccctcagtccaggcaagtga

SEQ ID NO: 19 - protein, 1634 aa, Artificial sequence
  von Willebrand Factor-Fc fusion polypeptide
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGISGGGGSGGGGSLVPRGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 75 bp, Artificial sequence
  furin and glycine-serine-glycine containing 2A sequence
cgtcgcaagcgcggaagtggagagggtcgcggcagccttctgacctgcggggatgtcgaagagaaccctggtccg

SEQ ID NO: 21 - DNA, 81 bp, Artificial sequence
  furin and glycine-serine-glycine containing 2A sequence
cgtcgcaagcgcggtagtggtggcgccaccaacttctccctgctgaagcaggccggcgacgtggaggagaaccccggcccc

SEQ ID NO: 22 - DNA, 4521 bp, Artificial sequence
  codon-optimized carbamoyl-phosphate synthase 1 coding sequence
atgcctcagatcataaagatgacccggattcttaccgcattcaaggttgtaaggacccttaaaaccggcttcggctttactaacgtgaccgcacaccaaaagtggaagtttagcaggcccggaattcgcctccttagtgtgaaagcccagaccgctcatatagtccttgaagacggcacaaaaatgaaagggtactcattcggccatccatcatctgtagccggtgaggtcgtgttcaatactggattggggggttatcccgaggccataacagacccagcttataagggccagatcctgaccatggccaacccaatcatcgggaacggaggtgcgccggatacaactgcgttggatgagctgggactgtccaagtacttggagagcaatggaattaaagtttctggactgctggtactggactactcaaaggactacaatcattggctggccaccaaaagtctggggcaatggctgcaggaggagaaggtgccagctatatacggagttgacactagaatgcttaccaaaattataagagacaaaggtactatgctgggaaaaattgagtttgaaggacagcccgtggatttcgtagaccctaataagcagaatcttatcgccgaggtgagcacaaaggacgttaaggtctacggaaaaggaaatccaactaaggtggtggctgttgattgtggcattaagaacaacgtgatcagactgctggtgaaacgcggagctgaagtccatcttgtcccatggaatcatgattttacgaaaatggagtatgatggaattctcatcgccggcggaccagggaacccagccttggctgaaccccttatccaaaacgttagaaaaatactcgaatctgataggaaagagcccctttttggtatatccaccggaaacttgattacaggccttgctgcaggggccaagacatataagatgagcatggcaaaccgcggacagaatcagcccgtactgaacattactaataagcaggcttttatcaccgcacagaatcacggttacgctctcgataatacgctccctgccggctggaagccgctcttcgttaacgtaaatgatcagacaaacgagggaataatgcacgaatccaaacccttcttcgccgtccagttccaccctgaagtcactccaggccctattgacacagaatatctctttgactccttctttagcctgataaaaaaggggaaggccaccaccataacgtccgtcctgcctaagccagctctcgtggcatcaagagtagaggtctccaaagtgctcatacttggtagcgggggactgtcaatcggccaagcaggcgagttcgattactccggaagccaagcagttaaggctatgaaagaagagaacgttaaaactgtgctgatgaatccaaatatagcctccgtgcagaccaatgaggtgggtctcaagcaagcagatactgtttactttcttccaattaccccccaattcgtaaccgaagtcattaaggccgagcagcctgatggattgatcctgggtatgggcggacagactgcactgaattgcggagtggagttgttcaaaaggggtgtgttgaaggaatatggagttaaggtactcggcacctccgttgagagcatcatggcgaccgaggatagacagttgttctctgataaactgaacgagattaatgagaagatcgccccctcattcgccgtggagtctatcgaagatgcactgaaagccgctgatacgattggctatcctgtaatgataagaagcgcctacgccctgggtggcctggggtctggcatctgccctaaccgagagacgctgatggacctctccacaaaagccttcgccatgactaaccagattctggtagaaaaatccgtcaccggctggaaggaaattgaatacgaagtagtaagagacgctgatgacaattgcgtcacagtctgcaacatggaaaacgtcgatgcgatgggcgtgcacaccggagattccgtcgttgtggcgccagcacaaacactctccaatgctgagttccagatgctcagaagaacaagcattaacgttgtgcgacatcttgggatagttggcgaatgtaacatccaatttgcactgcacccaactagcatggaatactgcattatcgaagtgaatgcgcggctgagccgaagcagcgctctcgccagcaaagccacaggctacccacttgccttcattgccgcaaagattgcactgggcattccactgcctgagattaagaatgtcgtaagcgggaagacaagcgcctgttttgaaccttccctggactatatggtgactaagattcctcggtgggaccttgataggttccatgggacctcatctagaataggatcatcaatgaagtctgtgggtgaagtgatggctatcgggcggaccttcgaagagagttttcagaaagcacttcggatgtgtcacccctcaattgagggcttcaccccccggttgccaatgaacaaggagtggccatcaaacctggacctgagaaaagagctcagcgagcctagctcaactagaatctacgcaatcgccaaggcaatcgacgataacatgtcattggatgagatagagaagttgacatacatagacaaatggttcctctacaaaatgcgagacattctgaatatggagaaaacactgaagggactgaattctgagagcatgacggaggagacacttaagagagcaaaagagattgggttcagcgataagcaaatttcaaagtgccttggactgaccgaagcccagacacgggagctgagactgaagaaaaatatacacccatgggtgaagcagatcgacaccctggcggccgaatatcccagcgttactaattacctgtatgttacatataacggccaagagcatgacgtaaattttgacgatcatggaatgatggttttgggatgcggtccctaccacattggctcttcagtggagtttgattggtgcgcagtgagctccattcggaccctcagacagcttggaaaaaaaacagtggtggtaaattgtaacccggagactgtgtcaaccgacttcgacgaatgcgacaagttgtattttgaggaattgagtcttgaaaggattcttgatatctaccatcaggaagcatgcggaggctgtattatctcagtgggcgggcagatacccaacaaccttgctgtacctctctataaaaacggtgtaaagatcatgggcacctctcccctccagattgacagggccgaggaccgctcaattttcagtgctgtgctggacgaactcaaagtcgctcaagctccttggaaagctgttaatactcttaacgaggccctcgagttcgccaagtctgtggattacccatgtcttcttcggccctcctacgtgctgtcaggatccgcaatgaacgtcgtgttcagcgaggatgaaatgaagaaatttctggaggaggctacacgggtgagtcaagagcatcctgtggttttgactaagttcgttgagggcgcccgggaagtcgagatggatgcagtcggtaaagatggacgggtaattagccacgcaattagtgaacacgtggaagatgccggggtccattctggcgacgccactctcatgctgccaacacagacaattagtcagggtgctatagagaaagtgaaagatgcgactaggaagatcgcaaaagccttcgcaatatctggcccatttaacgtgcagtttctcgtgaaaggtaacgacgtcctggtgatcgagtgtaatctccgagcgtcacgatccttccctttcgtaagcaagaccctcggcgtagactttattgacgtggccacgaaagttatgattggagagaatgtagacgagaaacacctccccactcttgaccatccgatcatccccgcggattatgttgccatcaaggccccaatgttctcttggccgcgcctgcgagacgctgatcccatcttgcgctgtgaaatggcaagcacaggcgaagtagcatgcttcggcgaaggtattcataccgcatttctgaaggccatgctgagcaccggcttcaagatcccccagaagggtatcctcatcggcatccagcagtctttccgcccaagattcctgggggtagcagaacaacttcataacgaaggcttcaagctgtttgcaacagaagcaacctctgattggctgaacgctaataatgttcctgcgactccagtcgcctggcccagccaggaaggacaaaatcccagcctgtctagcatcagaaaactcatacgagatggctctatcgaccttgttatcaacctgcctaataacaacaccaaatttgtccacgacaactacgtcatcagaagaactgccgtggatagcggtatccccctgctgaccaatttccaggttaccaagctctttgcagaagctgttcagaaatctcgcaaggtggatagcaagtcactgtttcactatcgacaatattcagcggggaaggctgcatag

SEQ ID NO: 23 - protein, 1506 aa, Artificial sequence
  codon-optimized carbamoyl-phosphate synthase 1 polypeptide
MPQIIKMTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 24 - DNA, 1605 bp, Artificial sequence
  codon optimized N-acetylglutamate synthase coding sequence
atggccacagctctgatggccgtggtcctgcgggccgctgccgttgcccctagactgcggggaagaggcgggacaggcggcgccagacggctgagctgtggcgcccggcggagagccgcccggggcaccagccctggcagaagactgagcaccgcctggagccaacctcagcctcctcctgaggaatacgccggagctgacgacgtgtcccagagccccgtggccgaggaaccctcttgggtcccttcccctagaccacccgtgcctcacgagagccccgagcctccaagcggaagaagcctggtgcagcgggacatccaggccttcctcaaccagtgcggcgcttcacctggagaggctagacactggctgacacagtttcagacctgtcaccactctgccgataagcctttcgccgtgatcgaggtggacgaggaagtgctgaagtgccagcaaggtgttagcagccttgcttttgccctggctttcctgcagagaatggacatgaagccgctggtggtgctgggcctgcctgcccctaccgccccatctggatgtctgtccttctgggaagccaaggcccagctggccaaaagctgcaaagtgctggtcgacgccctaagacacaacgccgccgctgccgtgcccttcttcggcggaggcagcgtgctgagggccgcagagcccgcccctcatgccagctatggcggcatcgtgtccgtggaaaccgacctcctgcaatggtgcctggaaagcggctctatccccattctgtgccctatcggcgagacagccgctaggagatccgtgctgctggattccctggaagtgaccgccagcctggctaaggccctccggcctacaaagatcatcttcctgaacaacaccggcggcctgagagacagcagccacaaggtgctgagcaatgtgaacctgcccgctgatctggacctggtgtgcaacgccgagtgggtgtctacaaaggaaagacagcagatgagactgatcgtggacgtgctgtccagactgccccatcacagcagcgccgtgatcacagccgcctctaccctgctgaccgagctgttcagcaacaagggcagcggcaccctgtttaagaacgccgagagaatgctgagagtgcggtcgctggacaagctggaccagggcagactggtggatctggtcaatgccagctttggaaaaaagctgagagatgactacctggcctctctgcggcctagactgcacagcatctacgtgtccgagggctacaacgccgccgcaatcctcaccatggaacccgtgctgggcggcaccccttacctggacaagttcgtggtgagcagctctagacagggccagggtagcggccagatgctgtgggagtgcctgagaagggacctgcagacactgttctggcggagccgggtgaccaaccctatcaacccctggtacttcaagcacagcgacggcagcttttctaataaacagtggatcttcttctggttcggcctggccgatatcagagatagctacgaactggtcaaccacgccaagggcctgccagacagcttccacaagcctgcctctgatcctggctcttga

SEQ ID NO: 25 - protein, 533 aa, Artificial sequence
  codon optimized N-acetylglutamate synthase polypeptide
ATALMAVVLRAAAVAPRLRGRGGTGGARRLSCGARRRAARGTSPGRRLSTAWSQPQPPPEEYAGADDVSQSPVAEEPSWVPSPRPPVPHESPEPPSGRSLVQRDIQAFLNQCGASPGEARHWLTQFQTCHHSADKPFAVIEVDEEVLKCQQGVSSLAFALAFLQRMDMKPLVVLGLPAPTAPSGCLSFWEAKAQLAKSCKVLVDALRHNAAAAVPFFGGGSVLRAAEPAPHASYGGIVSVETDLLQWCLESGSIPILCPIGETAARRSVLLDSLEVTASLAKALRPTKIIFLNNTGGLRDSSHKVLSNVNLPADLDLVCNAEWVSTKERQQMRLIVDVLSRLPHHSSAVITAASTLLTELFSNKGSGTLFKNAERMLRVRSLDKLDQGRLVDLVNASFGKKLRDDYLASLRPRLHSIYVSEGYNAAAILTMEPVLGGTPYLDKFVVSSSRQGQGSGQMLWECLRRDLQTLFWRSRVTNPINPWYFKHSDGSFSNKQWIFFWFGLADIRDSYELVNHAKGLPDSFHKPASDPGS

SEQ ID NO: 26 - DNA, 1065 bp, Artificial sequence
  codon optimized ornithine transcarbamylase coding sequence
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 27 - protein, 354 aa, Artificial sequence
  codon optimized ornithine transcarbamylase polypeptide
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 28 - DNA, 1290 bp, Unknown
  alpha-galactosidase A coding sequence
atgcagctccggaaccccgagctccaccttggctgcgccctcgccttgcggttcctcgcacttgtgagctgggacataccaggcgcccgggccctcgacaacggcctcgcccgcaccccaaccatgggctggctccactgggagcgcttcatgtgcaacctcgactgccaggaggagcccgactcctgcatctccgagaagcttttcatggagatggccgagctcatggtgtccgagggctggaaggacgccggctacgagtacctctgcatcgacgactgctggatggccccgcagcgcgacagcgagggtcgcctccaggccgacccgcagcggttccctcacggcatccgccagctcgccaactacgtccactccaagggcctcaagctcggcatctacgccgacgtcggcaacaagacctgcgccggcttccccggctccttcggctactacgacatcgacgcccagaccttcgccgactggggcgtcgacctcctcaagttcgacggctgctactgcgactccctcgagaacctcgccgacggctacaagcacatgtccctcgccctcaaccgcaccggccgctccatcgtctactcctgcgagtggcccctctacatgtggcccttccagaagcccaactacaccgagataaggcagtactgcaaccactggcgcaatttcgccgatatcgatgactcctggaagtccatcaagagcatcctggactggacctccttcaaccaggagcgcatcgtcgacgtcgccggccccggcggctggaacgaccccgacatgctcgtcatcggaaacttcggcctgtcctggaaccagcaggtcacccagatggccctctgggccatcatggccgccccactgttcatgtccaacgacctccgccacatcagcccgcaggccaaggccctcctccaggacaaggacgtcatcgccatcaaccaagacccgctcggcaagcagggctaccagctccgccagggcgacaacttcgaggtgtgggaacgtcccctcagcggcctggcgtgggccgtcgccatgatcaaccgccaggagatcggcggcccgcgctcctacaccatcgccgtggccagcctgggcaagggcgtcgcctgcaaccccgcctgcttcatcacccagctcctccccgtcaagagaaagctgggcttctacgagtggaccagccgcctccgctcccacatcaaccccaccggcaccgtcctgctccagctggagaacaccatgcagatgagcctcaaggacctgctctga

SEQ ID NO: 29 - protein, 429 aa, Unknown
  alpha-galactosidase A polypeptide
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 30 - DNA, 1290 bp, Artificial sequence
  alpha-galactosidase A coding sequence
atgcagctgcggaaccccgagctgcacctgggctgcgccctggccctgcggttcctggccctggtgagctgggacatccccggcgcccgggccctggacaacggcctggcccggacgcccaccatgggctggctgcactgggagcggttcatgtgcaacctggactgccaggaggagcccgacagctgcatcagcgagaagctgttcatggagatggccgagctgatggtgagcgagggctggaaggacgccggctacgagtacctgtgcatcgacgactgctggatggcgccccagcgggacagcgagggccggctgcaggccgacccgcagcggttccctcacggcatccggcagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaaccggaccggccggagcatcgtgtacagctgcgagtggcccctgtacatgtggcccttccagaagcccaactacaccgagatccggcagtactgcaaccactggcggaacttcgccgacatcgacgacagctggaagagcatcaagagcatcctggactggaccagcttcaaccaggagcggatcgtggacgtggccggccccggcggctggaacgaccccgacatgctggtgatcggcaacttcggcctgagctggaaccagcaggtgacccagatggccctgtgggccatcatggccgcgcccctgttcatgagcaacgacctgcggcacatcagccctcaggccaaggccctgctgcaggacaaggacgtgatcgccatcaaccaggacccactgggcaagcagggctaccagctgcggcagggcgacaacttcgaggtgtgggagcggcccctgagcggcctggcctgggccgtggccatgatcaaccggcaggagatcggcggcccgcggagctacaccatcgccgtggccagcctgggcaagggcgtggcctgcaaccccgcctgcttcatcacccagctgctgcccgtgaagcggaagctgggcttctacgagtggaccagccggctgcggagccacatcaaccccaccggcaccgtgctgctgcagctggagaacaccatgcagatgagcctgaaggacctgctgtga

SEQ ID NO: 31 - skipped

SEQ ID NO: 32 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 33 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 34 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 35 - protein, 476 aa, Artificial sequence
  B2M fused to an HLA-E fragment through a GS-linker
MAPRTLFLLLSGALTLTESGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 36 - protein, 500 aa, Artificial sequence
  HLA-G peptide joined via a linker to a B2M sequence joined via a linker to an HLA-E fragment
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 37 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 38 - protein, 293 aa, Artificial sequence
  truncated human CD47 sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 39 - DNA, 20 bp, Artificial sequence
  synthetic sequence
ggccacggagcgagacatct

SEQ ID NO: 40 - DNA, 20 bp, Artificial sequence
  synthetic sequence
cgcgagcacagctaaggcca

SEQ ID NO: 41 - DNA, 20 bp, Artificial sequence
  synthetic sequence
aagtcaacttcaatgtcgga

SEQ ID NO: 42 - DNA, 20 bp, Artificial sequence
  synthetic sequence
ggggccactagggacaggat

SEQ ID NO: 43 - DNA, 972 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 44 - skipped

SEQ ID NO: 45 - DNA, 882 bp, Artificial sequence
  truncated CD47 coding sequence
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggagtaa

SEQ ID NO: 46 - skipped

SEQ ID NO: 47 - DNA, 1503 bp, Artificial sequence
  B2M-HLA-E fusion coding sequence
atgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa